(19) United States
(12) Patent Application Publication
(10) Pub. No.: US 2018/0284123 A1
(43) Pub. Date: Oct. 4, 2018

(54) BARCODED RAPID ASSAY PLATFORM USEFUL FOR EFFICIENT ANALYSIS OF CANDIDATE MOLECULES AND METHODS OF MAKING AND USING THE PLATFORM

(71) Applicant: California Institute of Technology, Pasadena, CA (US)

(72) Inventors: James R. Heath, South Pasadena, CA (US); Amy M. McCarthy, Alameda, CA (US); Jungwoo Kim, Busan (KR)

(21) Appl. No.: 15/942,351

(22) Filed: Mar. 30, 2018

Related U.S. Application Data
(60) Provisional application No. 62/479,023, filed on Mar. 30, 2017.

(51) Int. Cl. G01N 33/58; G01N 33/50; G01N 33/68; G01N 33/552; G01N 33/483

(52) U.S. Cl. CPC G01N 33/582; G01N 33/5041; G01N 2458/10; G01N 33/552; G01N 33/4833; G01N 33/6812

(57) ABSTRACT

Disclosed are devices, compositions, and methods useful for assessing properties of compounds and molecules, such a binding, kinetic, and enzymatic properties, simultaneously for multiple compounds or molecules and/or under multiple conditions, efficiently, rapidly, and combinations of these. By using certain features alone or together in the save device or assay, the disclosed devices, compositions, and methods provide improvements over, and solve problems present in, prior assay devices and methods.

### CROSS-REFERENCE TO RELATED APPLICATIONS

This application claims benefit of and priority to U.S. Provisional Application No. 62/479,023, filed Mar. 30, 2017. Application No. 62/479,023, filed Mar. 30, 2017, is hereby incorporated herein by reference in its entirety.

### STATEMENT REGARDING FEDERALLY SPONSORED RESEARCH

This invention was made with Government support under Grant No. 1U54 CA199090-01 awarded by the National Cancer Institute, DGE-1144469 awarded by the National Science Foundation, and W911NF-09-D-0001 awarded by the Institute for Collaborative Biotechnologies (Department of Defense). The Government has certain rights in the invention.

### REFERENCE TO SEQUENCE LISTING

The Sequence Listing submitted Jun. 21, 2018, as a text file named “INDI34.1_US_ST25.txt,” created on Jun. 8, 2018, and having a size of 11,236 bytes is hereby incorporated by reference pursuant to 37 C.F.R. § 1.52(e)(5).

### FIELD OF THE INVENTION

The disclosed invention is generally in the field of assays and devices for assays and specifically in the area of high throughput multiplex assays and devices.

### BACKGROUND OF THE INVENTION

Protein-catalyzed capture agents (PCCs) have been demonstrated to mimic the epitope targeting ability and high avidity of monoclonal antibodies for a number of protein targets (Das et al., Angew. Chemie Int. Ed. 2015, 54 (45), 13219-13224). PCCs can be engineered to have combined properties that are difficult to achieve for biologics, such as combinations of physical and biological stability, or, in one example, cell penetration (Farrow et al., Angew. Chemie Int. Ed. 2015, 54 (24), 7114-7119). State-of-the-art PCCs are identified by carrying out an in situ click screen (Agnew et al., Angew. Chemie Int. Ed. 2009, 48 (27), 4944-4948) of a synthetic, strategically modified polypeptide fragment (the synthetic epitope, or SynEp) of the protein target against a synthetic one-bead-one compound (OBOC) library of macrocyclic peptides. The comprehensive OBOC library typically contains the roughly two million sequences that result from using all combinations of an 18-20 amino acid basis set to construct the variable 5-mer portion of the peptide.

PCC lead compounds are identified through a multi-step process, much of which is highly efficient. The OBOC library is first cleared of non-selective binders by screening against designated interferents. Candidate binders are then identified via a single generation in situ click screen against one or more SynEps of the targeted protein. That screen typically yields five to ten hits per SynEp. Once identified, those hit peptides are cleaved from the bead and sequenced using Edman degradation or mass spectrometry, prepared in ˜1 mg quantities, and then chromatographically purified. These steps are relatively efficient, and, with commercial robotics, can be accomplished in a few days. However, each PCC candidate must then be tested for binding to the full-length protein, often in various levels of serum background and under different blocking conditions. These assays are carried out on 96-well plates using a sandwich Enzyme-Linked Immunosorbent Assay (ELISA) format, and represent a limiting factor in PCC production.

Consider an in situ click screen in which a single OBOC library is screened against 2 SynEps to yield 15 PCC candidates. Each candidate is tested in, for example, a 10-point binding assay (run in triplicate) against the target protein. This yields 15×3×10=450 data points, which might be repeated for various background serum concentrations. In addition to being laborious, these assays also consume significant amounts of chemical and biological reagents. Finding a more efficient solution for carrying out such assays should be useful for the production of other artificial antibody-type ligands, such as other classes of peptides or aptamers (Yüce et al., Analyst 2015, 140 (16), 5379-5399; Jost and Plückthun, Current Opinion in Structural Biology. 2014, pp 102-112; Mascini et al., Angew. Chemie—Int. Ed. 2012, 51 (6), 1316-1332; Csordas et al., Anal. Chem. 2016, 88 (22), 10842-10847).

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF SUMMARY OF THE INVENTION

Disclosed are devices, compositions, and methods useful for assessing properties of compounds and molecules, such a binding, kinetic, and enzymatic properties, simultaneously for multiple compounds or molecules and/or under multiple conditions, efficiently, rapidly, and combinations of these. By using certain features alone or together in the save device or assay, the disclosed devices, compositions, and methods provide improvements over, and solve problems present in, prior assay devices and methods. For example, reduction in the time it takes to prepare and conduct assays can be produced by using smaller well volumes, which reduces the time needed for diffusion of reagents and reactants in the wells, by using detection reagent with a directly detectable label, which reduces the time needed to produce detectable signal, by patterning substrate oligomer paths on substrates, which reduces the time needed to produce assay substrates, and by assaying more molecules and more assay conditions on the same assay substrate, which reduces the number of assay substrates needed to assess a given number of molecules. These features can be used alone or in combination in any given device or assay, although the most benefit comes from combining them in the same device or assay.

Reliability of assay results can be increased by including each molecule to be assayed in each well, which makes the assay conditions for all of the molecules the same, by including replicates of each molecule to be assayed (and replicates of each control) in each well, which improves detectability of any real variability between the replicates by reducing random effects of assaying the replicates in different wells under (potentially different conditions), by including controls in each well, which makes the assay conditions for the controls the same as for the molecules to be assayed, and by use of a non-amplifying labelled detection reagent, which reduces noise and increases dynamic range. These features can be used alone or in combination in any given device or assay, although the most benefit comes from combining them in the same device or assay.

The amount and volume of molecules and reagents for the assays can be reduced by using micro scale of paths and wells, which reduces the cost, complexity, and difficulty in producing the reagents and, especially, the compounds or molecules to be assayed (which will often be new or unique compounds or molecules identified from libraries of compounds).

Disclosed are methods for producing devices embodying some or all of these useful features. In some forms, the methods involve contacting a solid substrate with a plurality of labelled candidate molecules, resulting in localization of each different labelled candidate molecule in a different path on the solid substrate, and, following or prior to contacting the solid substrate with the labelled candidate molecules, forming a plurality of test wells in the solid substrate, where one or more portions of each different path are in each well. In some forms of the method, the different labelled candidate molecules each comprise a different candidate molecule and a different label oligomer. In some forms of the method, the solid substrate comprises a plurality of the paths, where the paths are each positionally distinguishable and continuous. In some forms of the method, each of a plurality of different substrate oligomers is attached to a different one of the paths. In some forms of the method, each different label oligomer is complementary to a different one of the substrate oligomers. In some forms of the method, the label oligomers and the complementary substrate oligomers hybridize, where hybridization of a given label oligomer to the complementary substrate oligomer is bindingly distinguishable, which produces localization of each different labelled candidate molecule in a different one of the paths on the solid substrate.

In some forms of the method, each well can expose two or more different portions of each of the paths, where the two or more different portions of the paths are not continuous or contiguous in the well. In some forms of the method, each well exposes three different portions of each of the paths. In some forms of the method, the paths on the solid substrate change direction a plurality of times to form a serpentine pathway. In some forms of the method, one end of each path is proximal to a first side or edge of the solid substrate and the other end of each path is proximal to the side or edge of the solid substrate distal to the first side or edge of the solid substrate.

In some forms of the method, one or more of the paths constitutes a control path, wherein no labelled candidate molecule is localized in the control path. In some forms of the method, one or more of the control paths have a labelled control molecule localized in the control path, where the labelled control molecule is localized in the control path by, during the contacting step, contacting the solid substrate with the labelled control molecule. In some forms of the method, the labelled control molecule comprises a control molecule and a control label oligomer, where the control label oligomer is different from any of the label oligomers on the labelled candidate molecules localized on the solid substrate. In some forms of the method, the control label oligomer is complementary to one of the substrate oligomers, where the control label oligomer and the complementary substrate oligomer hybridize, resulting in localization of the control molecule in the path to which the complementary substrate oligomers is attached.

In some forms of the method, the paths can have a width of about 5 μm to about 100 μm. In some forms of the method, the paths can have a pitch of about 1.5 times to about 3 times the width of the paths. In some forms of the method, the paths can have a pitch of about 2 times the width of the paths. In some forms of the method, the width of the paths can be 50 μm. In some forms of the method, the paths can have a pitch of 100 μm.

In some forms of the method, each well can have an area of about 5 mm² to about 30 mm². In some forms of the method, each well can have an area of about 18 mm². In some forms of the method, the length of the shortest line that crosses all of the different paths in a wells can be about 450 μm to about 18 mm. In some forms of the method, the length of the shortest line that crosses all of the different paths in a well can be about 6 mm. In some forms of the method, the length of the shortest line that crosses the well can be about 150 μm to about 6 mm. In some forms of the method, the length of the shortest line that crosses the well can be about 3 mm.

In some forms of the method, the ratio of the length of the shortest line that crosses all of the different paths in a well and the length of the shortest line that crosses the well can be about 1 to about 5. In some forms of the method, the ratio of the length of the shortest line that crosses all of the different paths in a well and the length of the shortest line that crosses the well can be about 3. In some forms of the method, the solid substrate is rectangular. In some forms of the method, the solid substrate can comprise a glass slide or a plastic slide.

In some forms of the method, the method can further comprise coating the solid substrate with polylysine prior to attachment of the substrate oligomers to the solid substrate.

In some forms of the method, the solid substrate can comprise a bottom plate comprising a top surface, where the substrate oligomers are attached to the top surface of the bottom plate, where all of the paths are on the top surface of the bottom plate, and where the plurality of wells are formed by adhering a top plate to the top surface of the bottom plate. In some forms of the method, the top plate comprises perforations, where the wells comprise the surface of the bottom plate exposed by the perforations in the top plate. In some forms of the method, the top plate is a microchannel mold comprising the wells, where the wells are chambers over the surface of the bottom plate. In some forms of the method, the bottom plate is rectangular. In some forms of the method, the bottom plate is a glass slide or a plastic slide.

In some forms of the method, the method can further comprise coating the top plate with polylysine prior to attachment of the substrate oligomers to the solid substrate.

In some forms of the method, the method can further comprise, prior to contacting the solid substrate with the labelled candidate molecules and prior to forming the wells, adhering a microchannel mold onto the solid substrate, where the adhered microchannel mold forms a different continuous sealed channel above each path on the solid substrate; and flowing each different one of the substrate oligomers through a different formed channel and conjugating the substrate oligomers to the solid substrate.

In some forms of the method, contacting the solid substrate with the labelled candidate molecules can be accomplished by flowing the labelled candidate molecules through the formed channels. In some forms of the method, all of the labelled candidate molecules are flowed through each of the formed channels. In some forms of the method, each different one of the labelled candidate molecules is flowed through a different one of the formed channels. In some forms of the method, the method can further comprise, prior to forming the wells, removing the microchannel mold from the solid substrate. In some forms of the method, the microchannel mold can be fabricated from an elastomer. In some forms of the method, the wells can be formed prior to contacting the solid substrate with the labelled candidate molecules, where contacting the solid substrate with the labelled candidate molecules can be accomplished by adding all of the labelled candidate molecules to each of the wells. In some forms of the method, contacting the solid substrate with the labelled candidate molecules can be accomplished by adding all of the labelled candidate molecules to the solid substrate following removal of the microchannel mold and prior to forming the wells. In some forms of the method, contacting the solid substrate with the labelled candidate molecules can accomplished by adding all of the labelled candidate molecules to the solid substrate prior to forming the wells.

Also disclosed are devices for simultaneously testing a plurality of candidate molecules, the device comprising a solid substrate made by any of the disclosed methods that produce a device.

Also disclosed are methods of using the devices with attached candidate molecules and formed wells to assay the candidate molecules. In some forms of the method, the method involves adding an assay molecule to each well of the solid substrate, optionally excepting a control well, adding an imaging agent to each well of the solid substrate, where the imaging agent binds to the assay molecule or to a product of the assay molecule, the candidate molecule, or the assay molecule and candidate molecule together, and detecting the imaging agent on a plurality of the paths in a plurality of the wells.

In some forms of the method, the imaging agent can be detected in each of the paths in each of the wells. In some forms of the method, the imaging agent produces a fluorescent signal. In some forms of the method, the imaging agent produces a fluorescent signal upon excitation without the need for binding to or reaction with another molecule. In some forms of the method, the imaging agent is detected with a fluorescence image scanner. In some forms of the method, the image scanner generates a digitized output, where the digitized output is plotted as curves appropriate for the type of assay for each of the candidate molecules. In some forms of the method, the digitized output is plotted as binding curves for each of the candidate molecules. In some forms of the method, the imaging agent is detected in the middle third of the paths in the wells.

In some forms of the method, a measured value of the detected imaging agent is produced by averaging the signals of the imaging agent detected at different points along the paths in the wells. In some forms of the method, a measured value of the detected imaging agent is produced for a given path in a given well by averaging the signals of the imaging agent detected at different points along the given path in the given well. In some forms of the method, a measured value of the detected imaging agent is produced for a given candidate molecule in a given well by averaging the signals of the imaging agent detected on the different paths for the given candidate molecule in the given well.

In some forms of the method, the imaging agent comprises a fluorophore-labelled binding molecule. In some forms of the method, the imaging agent comprises a first binding molecule that binds to the assay molecule and a fluorophore-labelled binding molecule that binds to the first binding molecule. In some forms of the method, the first binding molecule is a primary antibody. In some forms of the method, the fluorophore-labelled binding molecule is a fluorophore-labelled antibody. In some forms of the method, the imaging agent comprises a fluorophore-labelled antibody. In some forms of the method, the imaging agent comprises a primary antibody that binds to the assay molecule, and a fluorophore-labelled antibody that binds to the primary antibody.

In some forms of the method, a different concentration of the assay molecule is added to each well of the solid substrate.

In some forms of the method, an interferent molecule is added to each well of the solid substrate, where the interferent molecule competes with the assay molecule for binding to the candidate molecules or inhibits reaction of the assay molecule with the candidate molecules. In some forms of the method, the interferent molecule is a competitive binding protein. In some forms of the method, a different concentration of the interferent molecule is added to each of the wells of the solid substrate.

In some forms of the method, both the label oligomers and the substrate oligomers are ssDNA molecules.

In some forms of the method, the labelled candidate molecules each further comprise a scaffold molecule, where the label oligomer of each labelled candidate molecule is chemically bonded to the scaffold molecule of the labelled candidate molecule and the candidate molecule of each labelled candidate molecule is bound or chemically bonded to the scaffold molecule of the labelled candidate molecule. In some forms of the method, the candidate molecule of each labelled candidate molecule is bound to the scaffold molecule of the labelled candidate molecule via a biotin-streptavidin interaction, where the scaffold molecule comprises streptavidin and the biotin is coupled to the candidate molecule. In some forms of the method, the label oligomer of each labelled candidate molecule is bound to the scaffold molecule of the labelled candidate molecule via a cysteine residue on the scaffold molecule.

In some forms of the method, 1 to 10 copies of the same label oligomer are bonded to each scaffold molecule. In some forms of the method, 2 to 4 copies of the same label oligomer are bonded to each scaffold molecule. In some forms of the method, 4 copies of the same label oligomer are bonded to each scaffold molecule. In some forms of the method, the label oligomers are modified via succinimide chemistry to have a 5′-aminated oligonucleotide. In some forms of the method, a hydrazide moiety is introduced to the candidate molecules via reaction with an amino group, where a hydrazine bond forms between the hydrazide moiety of the candidate molecules and the 5′-aminated oligonucleotide of the label oligomers.

In some forms of the method, the solid substrate comprises 10 paths to 30 paths. In some forms of the method, the solid substrate comprises 15 paths to 25 paths. In some forms of the method, the solid substrate comprises 20 paths. In some forms of the method, the solid substrate comprises 10 different candidate molecules to 30 different candidate molecules. In some forms of the method, the solid substrate comprises 15 different candidate molecules to 25 different candidate molecules. In some forms of the method, the solid substrate comprises 20 different candidate molecules.

Also disclosed are devices for simultaneously testing a plurality of candidate molecules. In some forms, the device comprises a solid substrate with a plurality of labelled candidate molecules and a plurality of test wells, where each different labelled candidate molecule is localized in a different path on the solid substrate, and where one or more portions of each different path are in each well. In some forms of the device, the different labelled candidate molecules each comprise a different candidate molecule and a different label oligomer. In some forms of the device, the solid substrate comprises a plurality of the paths, where the paths are each positionally distinguishable and continuous. In some forms of the device, each of a plurality of different substrate oligomers is attached to a different one of the paths. In some forms of the device, each different label oligomer is complementary to a different one of the substrate oligomers. In some forms of the device, the label oligomers and the complementary substrate oligomers are hybridized, where hybridization of a given label oligomer to the complementary substrate oligomer is bindingly distinguishable, which accounts for localization of each different labelled candidate molecule in a different one of the paths on the solid substrate.

In some forms of the device, each well exposes two or more different portions of each of the paths, where the two or more different portions of the paths are not continuous or contiguous in the well. In some forms of the device, each well exposes three different portions of each of the paths. In some forms of the device, the paths on the solid substrate change direction a plurality of times to form a serpentine pathway. In some forms of the device, one end of each path is proximal to a first side or edge of the solid substrate and the other end of each path is proximal to the side or edge of the solid substrate distal to the first side or edge of the solid substrate.

In some forms of the device, one or more of the paths constitutes a control path, wherein no labelled candidate molecule is localized in the control path. In some forms of the device, one or more of the control paths have a labelled control molecule localized in the control path. In some forms of the device, the labelled control molecule comprises a control molecule and a control label oligomer, where the control label oligomer is different from any of the label oligomers on the labelled candidate molecules localized on the solid substrate. In some forms of the device, the control label oligomer is complementary to one of the substrate oligomers, where the control label oligomer and the complementary substrate oligomer are hybridized, which accounts for localization of the control molecule in the path to which the complementary substrate oligomers is attached.

In some forms of the device, the paths can have a width of about 5 μm to about 100 μm. In some forms of the device, the paths can have a pitch of about 1.5 times to about 3 times the width of the paths. In some forms of the device, the paths can have a pitch of about 2 times the width of the paths. In some forms of the device, the width of the paths can be 50 μm. In some forms of the device, the paths can have a pitch of 100 μm.

In some forms of the device, each well can have an area of about 5 mm² to about 30 mm². In some forms of the device, each well can have an area of about 18 mm². In some forms of the device, the length of the shortest line that crosses all of the different paths in a wells can be about 450 μm to about 18 mm. In some forms of the device, the length of the shortest line that crosses all of the different paths in a well can be about 6 mm. In some forms of the device, the length of the shortest line that crosses the well can be about 150 μm to about 6 mm. In some forms of the device, the length of the shortest line that crosses the well can be about 3 mm.

In some forms of the device, the ratio of the length of the shortest line that crosses all of the different paths in a well and the length of the shortest line that crosses the well can be about 1 to about 5. In some forms of the device, the ratio of the length of the shortest line that crosses all of the different paths in a well and the length of the shortest line that crosses the well can be about 3. In some forms of the device, the solid substrate is rectangular. In some forms of the device, the solid substrate can comprise a glass slide or a plastic slide.

In some forms of the device, the solid substrate can comprise a bottom plate comprising a top surface, where the substrate oligomers are attached to the top surface of the bottom plate, where all of the paths are on the top surface of the bottom plate, and where the plurality of wells are formed by a top plate adhered to the top surface of the bottom plate. In some forms of the device, the top plate comprises perforations, where the wells comprise the surface of the bottom plate exposed by the perforations in the top plate. In some forms of the device, the top plate is a microchannel mold comprising the wells, where the wells are chambers over the surface of the bottom plate. In some forms of the device, the bottom plate is rectangular. In some forms of the device, the bottom plate is a glass slide or a plastic slide.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is a diagram showing the conventional assay for testing the binding of candidate ligands to a target.

FIG. 2 is a diagram of preparation of an example of a library of DNA-labelled cys-Streptavidin molecules. A streptavidin scaffold (20) is engineered with specific cysteine residues (labelled ‘c’). Single stranded (ss) DNA label oligomers (21; libomers) are chemically bonded to those sites using known chemistry to form a series of DNA-labelled cys-Streptavidin molecules (23). Each library element is defined by the DNA sequence of the label oligomers.

FIG. 3 is a diagram showing assembly of an example of a library of candidate ligand/DNA-labelled cys-Streptavidin complexes. Candidate ligands (30) are prepared with a biotin label (31) so that they will bind onto one of the 4 biotin binding sites on the DNA-labelled cys-Streptavidin molecules (23). The assembled complexes (32; labelled candidate ligand) are designed so that each candidate ligand has its own unique ssDNA oligomer (label oligomer) associated with it. Each of these library elements is defined by the combination of the candidate ligand and the DNA sequence of the label oligomer (21; FIG. 2).

FIG. 4 is a diagram of a surface that is spatially patterned with ssDNA′ oligomers (40). Each oligomer is complementary to one of the labels (21; FIG. 2) on the streptavidin scaffolds (20; FIG. 2). The library of candidate ligand/DNA-labelled cys Streptavidin complexes are all combined (41) and then introduced onto the DNA patterned surface, where each library element assembles onto a specific location (42) via DNA hybridization.

FIGS. 5A and 5B show diagrams of preparation of a Barcoded Rapid Assay Microchip. FIG. 5A shows the 50 μm barcode chip layout, which encompasses the entire length of a 3″ microscope slide. Input and outputs of the serpentine microchannels are at the right and left sides.

FIG. 6A is a picture of a barcoded rapid assay microchip (60) following its use for binding curve assays of KRAS to candidate ligands. A cutaway on the left shows the patterned substrate oligomers to which the labelled candidate ligands adhere. EC₅₀ binding curves of 15 candidate ligands were simultaneously generated against the target protein KRAS. Each microwell (ovals) represents a distinct concentration of KRAS that was added (values are given above each well). All 15 candidate ligands were measured in triplicate in each microwell.

FIG. 6B is a graph of the EC₅₀ binding curves generated from the barcoded rapid assay microchip of FIG. 6A. Images of the barcoded rapid assay microchip were digitized using a fluorescence image scanner and plotted as binding curves (61) for each of the candidate ligands. All binding curves were fitted to Hill functions, with the Hill coefficient set to 1. Table 2 shows the EC₅₀ values from the fitted curves, which fall into the 5 to 60 microMolar range, as expected.

FIG. 6C is a graph of the EC₅₀ binding curves generated from the barcoded rapid assay microchip of FIG. 6A. Images of the barcoded rapid assay microchip were digitized using a fluorescence image scanner and plotted as binding curves for each of the candidate ligands.

FIG. 7 shows solution loading device design (panel (i)) and flow patterning set ups with the solution loading device used in this work (panel (ii)) and the pins and tubing used for flow-patterning in previous work (panel (iii).

FIG. 8 is a graph of measured flow rates under various inlet pressures using the solution loading device.

FIG. 9 is a graph of the fluorescence measured in single point immunofluorescent assay on the barcoded rapid assay platform for the KRas allosteric switch ligands.

FIG. 10 is a graph of coefficient of variation (% CV) versus KRas concentration of assay results. The % CV for 1 μM to 400 μM KRas is shown, along with the average % CV for the entire concentration range (˜18%) (indicated by the black dashed line).

FIG. 11 is a graph comparing the fluorescence output (F₆₃₅ values) of replicate plates. The good correlation at low F₆₃₅ values indicate that the variance at low [KRas protein] is satisfactory.

FIG. 12 is a graph of the fluorescence of individual pixels across a barcode path (lane). This is a close up of the scatterplot of a barcode lane that highlights where the centroid region was defined and extracted (pixel 40-142) with the region divided into thirds for the left, middle, and right centroid respectively.

### DETAILED DESCRIPTION OF THE INVENTION

The disclosed method and compositions may be understood more readily by reference to the following detailed description of particular embodiments and the Example included therein and to the Figures and their previous and following description.

An outstanding challenge in the development of ligand binders is that most screening methods will generate a number of ‘hits,’ which are molecules that may have the desired binding properties against the target of interest. These molecules, called candidate ligands (a form of candidate molecule), must then be tested for binding affinity and selectivity against the target (often a protein), or the targeted region (e.g., epitope) of the protein, so as to determine which of the candidate ligands is the leading candidate. For example, one might have a set of candidate ligands, each of which needs to be tested in a full binding assay against the target, the epitope of the target, and under various conditions in which potential interferents are present. In such a binding assay, the candidate ligand is tested for binding against the target at various concentrations of either the target or the targeted region. Binding at each concentration value is measured in triplicate so as to generate reliable statistics, and multiple concentrations are similarly tested. For the testing of 10 candidate ligands, a full set of assays can include 1000 or more different measurements. Once all the measurements are done, the results are compiled to yield a series of binding curves that reveal how each candidate ligand interacts with the target or targeted epitope both with and without the presence of various interferents. One example might be the testing of how candidate ligands bind to a blood-borne protein in the presence of varying background levels of serum. The leading candidate will typically be the ligand that exhibits the best combination of high affinity and high selectivity for its target.

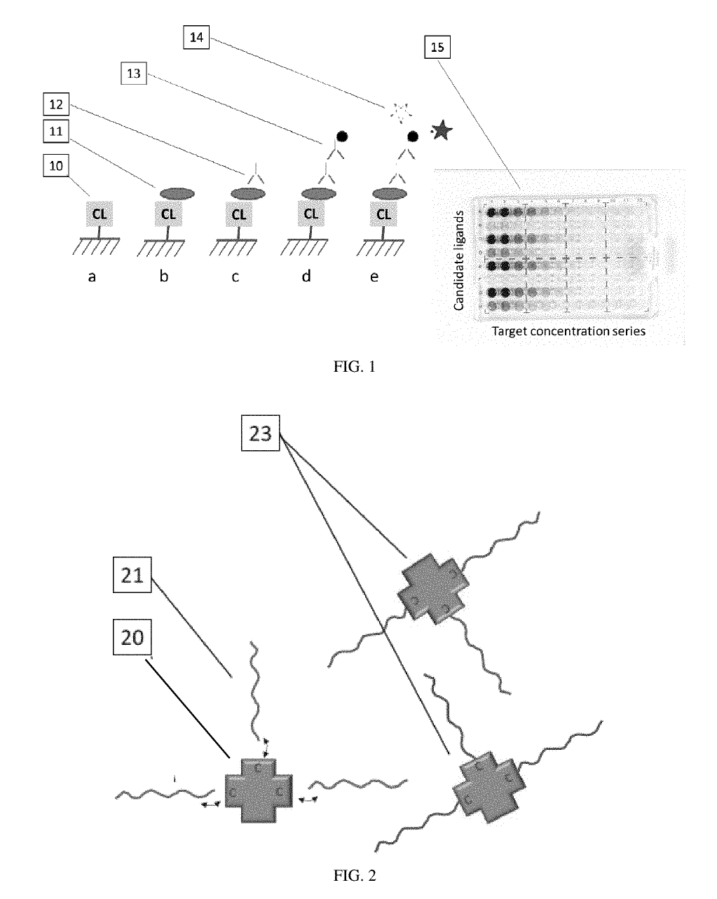

Such assays are typically carried out in series, using formats such as 96-well plates. FIG. 1 illustrates the conventional technique for making those measurements, with candidate ligands (10) surface immobilized. A known amount of target molecule (11) is added, some of which will bind to the candidate ligand. Next, an established binder to the target, such as an antibody (12) is added. The antibody provides a site for the binding of a second antibody (13), which is equipped with an enzyme. The enzyme is selected such that, when the substrate for the enzyme is provided, enzymatic processing of the substrate (14) will generate a colored product, which is then detected using optical absorbance. This last step is called ‘assay development.’ Typically all of these steps are done in a series of experiments using multi-well plate formats (15).

There are a few drawbacks with this current method, including the low sensitivity of the absorbance assay, and the relatively large amount of candidate ligands and target molecules that are consumed. Furthermore, slight variations in experimental procedure mean that many controls have to be run so as to provide confidence that the data from each candidate ligand can be accurately compared with that from the other candidate ligands. For example, in the plate (15) of FIG. 1, every other row is a control. A further drawback is that the actual assays can take time to run—ranging from a few hours to more than a day.

Protein Catalyzed Capture (PCC) Agents are an emergent class of macrocyclic peptides that selectively bind, with low nM affinities, to unique epitopes of specific proteins, such as oncoproteins and blood protein biomarkers. The development of a PCC binder is a robust process that can involve screening a two million elements one-bead-one-compound. (OBOC) macrocyclic peptide library against a target, such as a protein or an epitope of interest (see, for example, U.S. Pat. No. 8,906,830). A single generation screen is sufficient to identify candidate PCC ligands. Each candidate can then be tested via a series of binding assays to identify a PCC with optimal avidity characteristics. Such testing requires, for example, running multiple binding curve assays in series. It has been discovered that a newly designed high-throughput, microchip-based platform can provide for the rapid development of PCCs. In an example of such a platform, individual PCC candidates are prepared with a biotin label, coupled onto individual members of a DNA-encoded streptavidin library (DESL), and assembled, via DNA-hybridization, onto a micropatterned DNA barcode that is, or will be, located within a microwell of a multi-well chip. The exemplified platform allows for the simultaneous evaluation of fifteen different PCC ligands in sixteen different assay conditions on a single microchip (but this is not the limit for this platform). Additionally, the barcode surface chemistry reduces the non-specific background commonly seen in a sandwich ELISA (ELISA). The barcode platform was compared against traditional sandwich ELISA assays and was utilized to identify allosteric binders against the KRAS protein.

We report here on the barcoded rapid assay platform (B-RAP) (FIGS. 2-4), which is a microchip platform designed so that an entire set of candidate PCC ligands may be rapidly evaluated in parallel, using minimal quantities of reagents. Simultaneous testing of all PCCs under identical environments means that all assays are subject to the same uncertainties, which permits ready comparison of the EC₅₀ values for the entire set of hit peptides. The B-RAP technology draws from the Nucleic Acid Cell Sorting (NACS) (Kwong et al., J. Am. Chem. Soc. 2009, 131 (28), 9695-9703) and DNA-Encoded Antibody Library (DEAL) methods (Bailey et al., J. Am. Chem. Soc. 2007, 129 (7), 1959-1967; Boozer et al., Anal. Chem. 2004, 76 (23), 6967-6972; Kozlov et al., Biopolymers 2004, 73 (5), 621-630; Adler et al., Nat. Methods 2005, 2 (2), 147-149). The B-RAP process starts with a microscope slide that is patterned, using microfluidic flow channels, with a distinct set of orthogonal ssDNA oligomers. The PCC candidates are prepared with a biotin label, and then assembled onto cysteine-modified streptavidin (SAC) scaffolds that have been labelled with complementary ssDNA oligomers (Sano and Cantor, Proc. Natl. Acad. Sci. 1990, 87 (1), 142-146; Reznik et al., Bioconjug. Chem. 2001, 12 (6), 1000-1004; Ramachandiran et al., J. Immunol. Methods 2007, 319 (1-2), 13-20). Once assembled, these reagents are combined into a cocktail, and assembled onto specific stripes of the barcode pattern using DNA hybridization (Shin et al., ChemPhysChem 2010, 11 (14), 3063-3069). The microchip surface itself is partitioned into microliter volume wells, each of which contains multiple copies of the full barcode. The B-RAP technology can simultaneously assay a full panel of candidate PCCs over a range of target protein concentrations (or other conditions), such that the EC₅₀ binding values for each candidate PCC are concurrently measured.

Disclosed are devices, compositions, and methods useful for assessing properties of compounds and molecules, such a binding, kinetic, and enzymatic properties, simultaneously for multiple compounds or molecules and/or under multiple conditions, efficiently, rapidly, and combinations of these. By using certain features alone or together in the save device or assay, the disclosed devices, compositions, and methods provide improvements over, and solve problems present in, prior assay devices and methods. For example, reduction in the time it takes to prepare and conduct assays can be produced by using smaller well volumes, which reduces the time needed for diffusion of reagents and reactants in the wells, by using detection reagent with a directly detectable label, which reduces the time needed to produce detectable signal, by patterning substrate oligomer paths on substrates, which reduces the time needed to produce assay substrates, and by assaying more molecules and more assay conditions on the same assay substrate, which reduces the number of assay substrates needed to assess a given number of molecules. These features can be used alone or in combination in any given device or assay, although the most benefit comes from combining them in the same device or assay.

Reliability of assay results can be increased by including each molecule to be assayed in each well, which makes the assay conditions for all of the molecules the same, by including replicates of each molecule to be assayed (and replicates of each control) in each well, which improves detectability of any real variability between the replicates by reducing random effects of assaying the replicates in different wells under (potentially different conditions), by including controls in each well, which makes the assay conditions for the controls the same as for the molecules to be assayed, and by use of a non-amplifying labelled detection reagent, which reduces noise and increases dynamic range. These features can be used alone or in combination in any given device or assay, although the most benefit comes from combining them in the same device or assay.

The amount and volume of molecules and reagents for the assays can be reduced by using micro scale of paths and wells, which reduces the cost, complexity, and difficulty in producing the reagents and, especially, the compounds or molecules to be assayed (which will often be new or unique compounds or molecules identified from libraries of compounds).

One feature useful for the disclosed devices and methods is for each different candidate molecule (e.g., candidate ligand) to be present in each well multiple times (e.g., triplicate). It is also useful if controls are present in each well. These features result in the same assay conditions for all of the assays (i.e., all of the candidate molecules and their replicates) and all of the controls. This produces more reliable comparisons between the assay results for the different candidate molecules. Including each candidate molecule in each well exposes all candidate molecules to the same assay conditions resulting in more reliable comparisons between the assay results of different candidate molecules. Including replicates of each candidate molecule (and of each control) in each well results in more reliable assay results through statistical power of replicates. Including controls in each well exposes the controls to the same assay conditions as the candidate molecules resulting in more reliable values over control values.

Another feature useful for the disclosed devices and methods is for the surface regions (i.e., paths) that contain each candidate molecule (e.g., candidate ligand) are patterned to be microscopic. This allows small wells, the use of a small volume and amount of each candidate molecule and all of the reagents, and a reduced time of reaction (due to less time needed for diffusion of reagents over smaller well volume), while still allowing ten or more different candidate molecules to be present in each well (in, for example, triplicate). The micro scale of paths and wells results in (1) more candidate molecules tested per well, (2) reduced amounts and volumes of reagents, and (3) reduced time for assay).

Another feature useful for the disclosed devices and methods is to use a detectable label attached to a detection reagent (e.g., detection antibody), which can reduce the time to produce a detectable signal at the site of the object of detection compared to detection methods requiring time for colorimetric change, such as enzymatic reactions to produce a label. Use of a labelled detection reagent (i.e., non-amplified label) (1) reduces the time for the assay by eliminating the time needed for development of a label and (2) allows reliable evaluation of weakly active (e.g., weakly binding) candidate molecules by reduction of noise in the label detection and by providing a higher dynamic range of label signal.

Another feature useful for the disclosed devices and methods is to use microscale paths of substrate oligomers microfabricated in a serpentine pattern, which allows some the benefits discussed above by a single input point for each substrate oligomer on each substrate. This allows automated and high throughput production of devices and solid substrates. Efficient production of devices and solid substrate increases overall efficiency and throughput of assay systems using the substrates. This also allows replicates to be included in wells by the simple expedient of having the well overlay multiple rows of paths (a row of paths being or representing a full barcode).

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a candidate molecule is disclosed and discussed and a number of modifications that can be made to a number of molecules including the candidate molecule are discussed, each and every combination and permutation of candidate molecule and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

As used herein, the term “non-natural amino acid” refers to an organic compound that has a structure similar to a natural amino acid so that it mimics the structure and reactivity of a natural amino acid. The non-natural amino acid as defined herein generally increases or enhances the properties of a peptide (e.g., selectivity, stability) when the non-natural amino acid is either substituted for a natural amino acid or incorporated into a peptide.

As used herein, the term “peptide” refers to a class of compounds composed of amino acids chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides.

As used herein, the term “activity” refers to a biological activity.

As used herein, the term “pharmacological activity” refers to the inherent physical properties of a peptide or polypeptide. These properties include but are not limited to half-life, solubility, and stability and other pharmacokinetic properties.

The term “positionally distinguishable” as used herein, refers to objects that are distinguishable based on the point or area occupied by the objects. Accordingly, positionally distinguishable paths are paths that occupy different points or areas on a solid substrate and are thereby positionally distinguishable. Similar, positionally distinguishable scaffold binding domains are binding domains that occupy different points or areas on the scaffold and are thereby positionally distinguishable.

The term “bindingly distinguishable” as used herein with reference to molecules, such as oligomers, indicates molecules that are distinguishable based on their ability to specifically bind to, and are thereby defined as complementary to, a specific molecule. Accordingly, a first molecule is bindingly distinguishable from a second molecule if the first molecule specifically binds and is thereby defined as complementary to a third molecule and the second molecule specifically binds and is thereby defined as complementary to a fourth molecule, with the fourth molecule distinct from the third molecule. Accordingly, a first and second label oligomers are bindingly distinguishable, if the first label oligomer specifically binds (and is thereby defined as complementary) to a first substrate oligomer and the second label oligomer specifically binds (and is thereby defined as complementary to) a second substrate oligomer, with the first substrate oligomer distinct from the second substrate oligomer.

The wording “detectably distinguishable” as used herein with reference to labelled molecule indicates molecules that are distinguishable on the basis of the labeling signal provided by the label compound attached to the molecule. Exemplary label compounds that can be use to provide detectably distinguishable labelled molecules, include but are not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and additional compounds identifiable by a skilled person upon reading of the present disclosure.

The term “attach” or “attached” as used herein, refers to connecting or uniting by a bond, link, force or tie in order to keep two or more components together, which encompasses either direct or indirect attachment such as, embodiments where a first molecule is directly bound to a second molecule or material, and embodiments wherein one or more intermediate molecules are disposed between the first molecule and the second molecule or material. Molecules include but are not limited to oligomers, polypeptides, and in particular proteins and antibodies, polysaccharides, aptamers and small molecules.

The term “oligomer” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base, and to a phosphate group and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group.

The term “polypeptide” as used herein indicates an organic polymer composed of two or more amino acid monomers and/or analogs thereof. The term “polypeptide” includes amino acid polymers of any length including full length proteins and peptides, as well as analogs and fragments thereof. A polypeptide of three or more amino acids is also called a protein oligomer or oligopeptide. As used herein the term “amino acid”, “amino acidic monomer”, or “amino acid residue” refers to any of the twenty naturally occurring amino acids including synthetic amino acids with unnatural side chains and including both D an L optical isomers. The term “amino acid analog” refers to an amino acid in which one or more individual atoms have been replaced, either with a different atom, isotope, or with a different functional group but is otherwise identical to its natural amino acid analog.

The term “protein” as used herein indicates a polypeptide with a particular secondary and tertiary structure that can participate in, but not limited to, interactions with other biomolecules including other proteins, such as antibodies, DNA, RNA, lipids, metabolites, hormones, chemokines, and small molecules.

The term “antibody” as used herein refers to a protein that is produced by activated B cells after stimulation by an antigen and binds specifically to the antigen promoting an immune response in biological systems and that typically consists of four subunits including two heavy chains and two light chains. The term antibody includes natural and synthetic antibodies, including but not limited to monoclonal antibodies, polyclonal antibodies or fragments thereof. Exemplary antibodies include IgA, IgD, IgG1, IgG2, IgG3, IgM and the like. Exemplary fragments include Fab Fv, Fab′ F(ab′)2 and the like. A monoclonal antibody is an antibody that specifically binds to and is thereby defined as complementary to a single particular spatial and polar organization of another biomolecule which is termed an “epitope”. A polyclonal antibody refers to a mixture of monoclonal antibodies with each monoclonal antibody binding to a different antigenic epitope. Antibodies can be prepared by techniques that are well known in the art, such as immunization of a host and collection of sera (polyclonal) or by preparing continuous hybridoma cell lines and collecting the secreted protein (monoclonal).

The wording “specific”, “specifically”, or specificity” as used herein with reference to the binding or attachment of a molecule to another refers to the recognition, contact and formation of a stable complex between the molecule and the another, together with substantially less to no recognition, contact and formation of a stable complex between each of the molecule and the another with other molecules. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of. The term “specific” as used herein with reference to a sequence of a polynucleotide refers to the unique association of the sequence with a single polynucleotide which is the complementary sequence.

The term “detect” or “detection” as used herein indicates the determination of the existence, presence or fact of a target or signal in a limited portion of space, including but not limited to a sample, a reaction mixture, a molecular complex and a substrate. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “target” as used herein indicates an analyte of interest. The term “analyte” refers to a substance, compound or component whose presence or absence in a sample has to be detected. Analytes include but are not limited to biomolecules and in particular biomarkers. The term “biomolecule” as used herein indicates a substance compound or component associated to a biological environment including but not limited to sugars, amino acids, peptides proteins, oligonucleotides, polynucleotides, polypeptides, organic molecules, haptens, epitopes, biological cells, parts of biological cells, vitamins, hormones and the like. The term “biomarker” indicates a biomolecule that is associated with a specific state of a biological environment including but not limited to a phase of cellular cycle, health and disease state. The presence, absence, reduction, upregulation of the biomarker is associated with and is indicative of a particular state.

Also disclosed are devices for simultaneously testing a plurality of candidate molecules. In some forms, the device comprises a solid substrate with a plurality of labelled candidate molecules and a plurality of test wells, where each different labelled candidate molecule is localized in a different path on the solid substrate, and where one or more portions of each different path are in each well. In some forms of the device, the different labelled candidate molecules each comprise a different candidate molecule and a different label oligomer. In some forms of the device, the solid substrate comprises a plurality of the paths, where the paths are each positionally distinguishable and continuous. In some forms of the device, each of a plurality of different substrate oligomers is attached to a different one of the paths. In some forms of the device, each different label oligomer is complementary to a different one of the substrate oligomers. In some forms of the device, the label oligomers and the complementary substrate oligomers are hybridized, where hybridization of a given label oligomer to the complementary substrate oligomer is bindingly distinguishable, which accounts for localization of each different candidate molecule in a different one of the paths on the solid substrate.

In some forms of the device, each well exposes two or more different portions of each of the paths, where the two or more different portions of the paths are not continuous or contiguous in the well. In some forms of the device, each well exposes three different portions of each of the paths. In some forms of the device, the paths on the solid substrate change direction a plurality of times to form a serpentine pathway. In some forms of the device, one end of each path is proximal to a first side or edge of the solid substrate and the other end of each path is proximal to the side or edge of the solid substrate distal to the first side or edge of the solid substrate.

In some forms of the device, one or more of the paths constitutes a control path, wherein no labelled candidate molecule is localized in the control path. In some forms of the device, one or more of the control paths have a labelled control molecule localized in the control path. In some forms of the device, the labelled control molecule comprises a control molecule and a control label oligomer, where the control label oligomer is different from any of the label oligomers on the labelled candidate molecules localized on the solid substrate. In some forms of the device, the control label oligomer is complementary to one of the substrate oligomers, where the control label oligomer and the complementary substrate oligomer are hybridized, which accounts for localization of the control molecule in the path to which the complementary substrate oligomers is attached.

In some forms of the device, the paths can have a width of about 5 μm to about 100 μm. In some forms of the device, the paths can have a pitch of about 1.5 times to about 3 times the width of the paths. In some forms of the device, the paths can have a pitch of about 2 times the width of the paths. In some forms of the device, the width of the paths can be 50 μm. In some forms of the device, the paths can have a pitch of 100 μm.

In some forms of the device, each well can have an area of about 5 mm² to about 30 mm². In some forms of the device, each well can have an area of about 18 mm². In some forms of the device, the length of the shortest line that crosses all of the different paths in a wells can be about 450 μm to about 18 mm. In some forms of the device, the length of the shortest line that crosses all of the different paths in a well can be about 6 mm. In some forms of the device, the length of the shortest line that crosses the well can be about 150 μm to about 6 mm. In some forms of the device, the length of the shortest line that crosses the well can be about 3 mm.

In some forms of the device, the ratio of the length of the shortest line that crosses all of the different paths in a well and the length of the shortest line that crosses the well can be about 1 to about 5. In some forms of the device, the ratio of the length of the shortest line that crosses all of the different paths in a well and the length of the shortest line that crosses the well can be about 3. In some forms of the device, the solid substrate is rectangular. In some forms of the device, the solid substrate can comprise a glass slide or a plastic slide.

In some forms of the device, the solid substrate can comprise a bottom plate comprising a top surface, where the substrate oligomers are attached to the top surface of the bottom plate, where all of the paths are on the top surface of the bottom plate, and where the plurality of wells are formed by a top plate adhered to the top surface of the bottom plate. In some forms of the device, the top plate comprises perforations, where the wells comprise the surface of the bottom plate exposed by the perforations in the top plate. In some forms of the device, the top plate is a microchannel mold comprising the wells, where the wells are chambers over the surface of the bottom plate. In some forms of the device, the bottom plate is rectangular. In some forms of the device, the bottom plate is a glass slide or a plastic slide.

Disclosed are devices for simultaneously testing a plurality of candidate molecules, the device comprising a solid substrate made by any of the disclosed methods that produce a device.

Useful features the devices and solid substrates include forms and materials that allow all of the paths of attached substrate oligomers to be produced simultaneously, preferably with a single impetus for patterning the paths of the solid substrate. An example of such a useful feature is a microchannel mold adhered to the surface of the solid substrate, where the microchannel mold has channels for each path and ends of the channels that allow different substrate oligomers to be loaded in different paths but also allowing a single manifold with a single opening to be used to apply flow pressure to loaded substrate oligomers at the same time.

Another example of useful features of the devices and solid substrates is sets of substrate oligomers and label oligomers that are bindingly distinguishable (i.e., are orthogonal). This allows a single solution of all of the labelled candidate molecules to be contacted with the solid substrate for attachment to their intended and distinct paths.

Another example of useful features of the devices and solid substrates is patterning of the paths so that wells with small areas can overlap with multiple sets of the paths (e.g., multiple full barcodes of paths). Examples of such useful features include the multiple paths being parallel to adjacent paths, such as paths that make turns together resulting in nested turns, with the outer path in the set of paths turning back immediately adjacent to the outer path and the inner path turning outside and around all of the other paths in the set. The effect is to produce multiple full barcodes of paths traversing the surface parallel to other full barcodes of paths traversing the surface. FIG. 5 shows an example of a preferred pattern of paths.

The disclosed devices and solid substrates include any of the intermediate forms of the devices and solid substrates. This includes, for example, solid substrates, such as bottom plates, with material or components, such as polylysine, for attachment of the substrate oligomers to the solid substrate. Such a solid substrate could be packaged in kits or stored for later use, for example. Another example is solid substrates, such as bottom plates, with material or components, such as microchannel molds, for patterned flow and attachment of the substrate oligomers to the solid substrate. Such a solid substrate could be packaged in kits or stored for later use, for example.

Another example is solid substrates, such as bottom plates, with material or components, such as substrate oligomers, attached to the solid substrate in a useful pattern. Such a solid substrate could be packaged in kits or stored for later use, for example. Such a solid substrate is a preferred form of solid substrate for inclusion in kits, allowing users to attach candidate molecules of their choice to the solid substrate. Such a solid substrate can have the patterning component (e.g., microchannel mold) still adhered to the surface or such a solid substrate can be without the patterning component.

Another example is solid substrates, such as bottom plates, with material or components, such as candidate molecules, attached to the solid substrate in a useful pattern. Such a solid substrate could be packaged in kits or stored for later use, for example. Such a solid substrate can have the patterning component (e.g., microchannel mold) still adhered to the surface or such a solid substrate can be without the patterning component.

Another example is solid substrates, such as bottom plates, with material or components, such as top plates, attached to the solid substrate to form wells. Such a solid substrate could be packaged in kits or stored for later use, for example. Such a solid substrate is another preferred form of solid substrate for inclusion in kits, allowing users to just perform assays using a solid substrate having defined candidate molecules attached to the solid substrate. In some forms, sets of such solid substrates can be provided that include a library of candidate molecules for use in assays.

Another example is solid substrates having wells and attached candidate molecules with material or components, such as imaging agents, present in the wells. Such a solid substrate could be packaged in kits or stored for later use, for example.

Another example is solid substrates having wells, attached candidate molecules, with material or components, such as assay molecules, present in the wells. In some forms, this can be an intermediate or final product of an assay method.

Another example is solid substrates having wells, attached candidate molecules, assay molecules attached to the candidate molecules, with material or components, such as imaging agents, present in the wells. In some forms, this can be an intermediate or final product of an assay method.

Another example is solid substrates having wells, attached candidate molecules, assay molecules attached to the candidate molecules, and imaging agents attached to the assay molecules. In some forms, this can be a final product of an assay method.

### A. Substrate Oligomers and Label Oligomers

Substrate oligomers and label oligomers are used in the disclosed devices and methods to orthogonally associate components attached to a given label oligomer to that label oligomer's complementary substrate oligomer. When the substrate oligomers are attached to a solid substrate, hybridization of a label oligomer to its complementary substrate oligomer localizes the label oligomer (and components attached to the label oligomer) to the site(s) where the complementary substrate oligomer is attached to the solid substrate. In the main forms of the disclosed devices, the different substrate oligomers are attached to the solid substrate to form different paths. Thus, in some forms of the device, each of a plurality of different substrate oligomers is attached to a different one of the paths. In some forms of the device, each different label oligomer is complementary to a different one of the substrate oligomers. In some forms of the device, the label oligomers and the complementary substrate oligomers are hybridized, where hybridization of a given label oligomer to the complementary substrate oligomer is bindingly distinguishable, which accounts for localization of each different candidate molecule in a different one of the paths on the solid substrate.

The term “substrate oligomer” as used herein refers to a polynucleotide that is attached to a solid substrate so to maintain the ability to bind to its complementary polynucleotide. A substrate oligomer can be, in particular, comprised of a sequence that specifically binds and is thereby defined as complementary with an encoding-polynucleotide of a polynucleotide encoded protein.

The term “solid substrate” as used herein indicates an underlying support or substratum. Exemplary solid substrates include glass plates, microtiter well plates, magnetic beads, silicon wafers and additional solid substrates identifiable by a skilled person upon reading of the present disclosure.

The term “orthogonalization” as used herein refers to the process by which a set of polynucleotides or oligomers are generated computationally, in which incomplete base pairing, metastable states and other secondary structures are minimized, such that a polynucleotide only binds to its complementary strand and none other. Exemplary orthogonalization techniques used in this disclosure include orthogonalization performed according to the paradigm outlined by Dirks et al., Nucleic Acids Research 2004, 32, (4), 1392-1403. In particular, in some embodiments, the label oligomers and the corresponding complementary substrate oligomers are orthogonalized polynucleotides such as polynucleotides in Table 5.

The disclosed oligomers can be made of any form of nucleotide, nucleotide analog, or polynucleotide analog that allows specific base interactions. So long as their relevant function is maintained, oligomers and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH₂)nO]m CH₃, —O(CH₂)nOCH₃, —O(CH₂)nNH₂, —O(CH₂)nCH₃, —O(CH₂)n-ONH₂, and —O(CH₂)nON[(CH₂)nCH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs can also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides and nucleic acids can be referred to as chimeric oligonucleotides and chimeric nucleic acids.

### B. Candidate Molecules

Candidate molecules can be any molecule that is believed, suspected, or known to bind to or otherwise interact with an assay molecule or interests, such as a target molecule. Labelled candidate molecule preferably comprise a scaffold molecule that connects the candidate molecule to a label oligomer.

Candidate molecules are any compound or molecule to be assessed for the properties related to an assay molecule. In preferred forms, candidate molecules are molecules that are candidates for binding to or interacting with or on an assay molecule. Most preferable, the candidate molecules are candidates for binding to an assay molecule. This last form is extensively explored in the examples where the binding affinity of candidate molecules to a target protein are assessed using examples of the disclosed devices and methods. Preferred candidate molecules are PCCs and candidate ligands for use in PCC.

Because the disclosed devices and methods constitute a broadly applicable platform and system for assessing any set of candidate molecules of any type for a variety of properties and activities in response to any assay molecule of interest, there is no a priori limit to which candidate molecules and which assay molecules can be used with the disclosed devices and methods (except that the property or interaction to be assessed must be capable of assessment using the disclosed devices and methods.

Preferred candidate molecules are molecules, such as compounds, peptides, and aptamers, which are candidates for specifically binding to an assay molecule. The disclosed devices and methods are particularly useful for screening candidate molecules for specific and high affinity binding to such target molecules.

The term “scaffold” or “scaffold molecule” as used herein indicates a molecular structure of a labelled candidate molecule that serves to assemble a candidate molecule to a label oligomer (e.g., ssDNA tags). This structure can be derived from proteins (such as Streptavidin or SA), other biopolymers (such as polynucleotides, like RNA and DNA, peptide nucleic acid, etc.), or other polymers which can bind to the candidate molecule and the label oligomer in distinct and separate portions of the polymer.

In some forms of the labelled candidate molecules, the scaffold molecule can be configured to bind the candidate molecule and a label oligomer with scaffold binding domains.

The term “domain” as used herein with indicates a region that is marked by a distinctive structural and functional feature. In particular, a scaffold binding domain is a region of the scaffold that is configured for binding with another molecule. Accordingly, a scaffold binding domain in the sense of the present disclosure includes a functional group for binding the other molecule and a scaffold binding region on the scaffold that is occupied by the another molecule bound to the scaffold. Once the functional group has been identified, the relevant scaffold binding region can be determined with techniques suitable to identify the size and in particular the largest diameter of the other molecule of choice to be attached. The average largest diameter for a protein according to the present disclosure in several embodiments is between about 10 Å and about 50 Å depending on the protein of choice, between about 3 Å and about 10 Å for a small molecule, and is between about 10 Å and about 20 Å for a polynucleotide. Techniques suitable to identify dimensions of a molecule include but are not limited to X-ray crystallography for molecules that can be crystallized and techniques to determine persistence length for molecules such as polymers that cannot be crystallized. Those techniques for detecting a molecule dimensions are identifiable by a skilled person upon reading of the present disclosure.

In some forms of the labelled candidate molecules, the scaffold binding domains can be positionally distinguishable among each other, and therefore, do not overlap.

The term “present” as used herein with reference a molecule or portion thereof, (e.g., a functional group or a restriction site) that has a chemical reactivity and is comprised in a structure, indicates a configuration of the molecule or functional group in the structure wherein the molecule or portion thereof maintains a detectable level of such chemical reactivity. Accordingly, a molecule or a functional group presented on a scaffold is a molecule or portion thereof comprised in that scaffold in a configuration that allows performing, and detecting, under the appropriate conditions, the one or more chemical reactions that chemically and/or biologically characterize the molecule or portion thereof at issue.

Therefore in labelled candidate molecules comprising a scaffold, upon attachment of the candidate molecule and the label oligomer with the scaffold, the candidate molecule is presented for binding to the assay molecule and the label oligomer is presented for binding to a substrate oligomer.

In some forms of the labelled candidate molecules, presentation of the candidate molecule and label oligomer on the scaffold is achieved by selecting a scaffold with appropriate first and second scaffold binding domains.

Functional groups for binding a candidate molecule, that can be included in a first scaffold binding domain, depend on the chemical nature of the candidate molecule and are identifiable by the skilled person upon reading of the present disclosure. For example, functional groups for binding a candidate molecule include but are not limited to BirA Ligase (enzyme that attaches biotin group to predefined peptide sequences), other enzymes such as formylglycine-generating enzyme.

Functional groups for binding a polynucleotide, that can be included in a second scaffold binding domain, are also identifiable by the skilled person upon reading of the present disclosure. Exemplary functional groups presented on the scaffold for binding a polynucleotide include functional groups such as sulfulhydryl (e.g. in a cysteine residue), primary amines and other functional groups that attach derivatized DNA via conventional conjugation strategies, that would be identifiable by the skilled reader.

Those functional groups can either be endogenous groups on the scaffold (e.g. native lysine residues on a scaffold protein), or introduced by methods such as gene cloning (e.g. proteins), synthetic techniques (polymers, small molecules), and other methods. The number of copies of polynucleotides or candidate molecules that can attach to the scaffold will be directly proportional to the number of functional groups available on the scaffold.

The specific first and second functional groups and related scaffold binding domain are selected in view of the experimental design. Usually, the scaffold is selected so that the functional groups of the first and second scaffold binding regions allow attachment of the candidate molecule and the label oligomer using orthogonal chemistries. A set of attachment chemistries is orthogonal if, when performing any particular chemistry, the functional groups that participate and/or undergo a chemical reaction in that particular chemistry do not react with any other chemistry within the orthogonal set. Exemplary orthogonal chemistries include cysteine-maleimide coupling, amine-NHS coupling, and streptavidin-biotin binding, when a scaffold is a protein, and controlled oxidization of OH functional groups in different scaffold binding regions with NaIO₄ when the scaffold is a polysaccharide.

In some forms, in addition to containing distinct scaffold binding domains to accommodate the candidate molecule and label oligomer, the scaffold is also selected to be compatible with the environment of the target of interest.

In some forms, the scaffold is provided by a non-naturally occurring molecule that is expressed with modular design characteristics. In those embodiments, the protein scaffold is designed so that multiple and controlled numbers of copies of specific candidate molecules and label oligomers may be attached to the scaffold at specific scaffold polynucleotide binding domains.

In some forms, the scaffold can be configured to enable or ease attachment of multiple copies of single-stranded label oligomer (e.g. DNA oligomers) in multiple second scaffold binding domains. In those forms, the second scaffold binding domain can be selected to allow hybridization with an label oligomer to be used to spatially direct the scaffold to particular spots on a surface that are coated with the substrate oligomers.

In some embodiments, a desired configuration of a scaffold and, in particular, a scaffold protein, can be achieved through modification of candidate scaffolds that are modified with techniques known to the skilled person such as traditional cloning techniques or other techniques identifiable by a skilled person.

### C. Solid Substrates

The disclosed devices comprise solid substrates to which various components are or have been attached. The term “solid substrate” as used herein indicates an underlying support or substratum. Exemplary solid substrates include glass plates, microtiter well plates, magnetic beads, silicon wafers and additional solid substrates identifiable by a skilled person upon reading of the present disclosure.

Solid substrates can include any solid material with which components can be associated, directly or indirectly. This includes materials such as acrylamide, agarose, carboxylated poly(vinyl chloride) (CPVC), cellulose acetate membrane, cellulose nitrate (CN) membrane, cellulose, collagen, filter paper (Whatman), fluorocarbons, functionalized silane, Glass fiber filters (GFC) (A,B,C), glass, glycosaminoglycans, gold, latex, mixed cellulose ester membrane, nitrocellulose, nylon, plastic, polyamino acids, polyanhydrides, polycarbonates, polyethersulfone (PES) membrane, polyethylene oxide, polyethylene vinyl acetate, polyethylene, polyethylimine coated GFCs, polyglycolic acid, polylactic acid, polymethacrylate, polyorthoesters, polypropylene, polypropylfumerate, polysilicates, polystyrene, polyvinylidene fluoride (PVDF), porous mylar or other transparent porous films, PTFE membrane, silicon rubber, teflon, andultrafiltration membranes of poly(vinyl chloride) (PVC). Solid-state substrates can have any useful form including beads, bottles, chemically-modified glass slides, column matrix, cross-linked polymer beads, dishes, fibers, mass spectrometer plates, membranes, microparticles, microtiter dishes, particles, shaped polymers, slides, sticks, test strips, thin films, thin membranes, and woven fibers, or a combination. Solid-state substrates and solid substrates can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

In some forms, the solid substrate comprises a top plate and a bottom plate. Each plate has a top surface, a bottom surface, and an edge. The shape and linear dimensions the top plate can be substantially the same as the shape and linear dimensions of the bottom plate such that edges of the top and bottom plates can be aligned when the top plate is affixed to the bottom plate (except that, optionally, all or a portion of the edge of the top plate can be recessed from the edge of the top plate). The thickness of a plate from the top surface to the bottom surface is substantially the same across the plate (which can be referred to as the plate being substantially planar). The smallest dimension across the surfaces of a plate can have a ratio with the thickness of the plate of greater than 10 to greater than 24.

The edge of a plate can be continuous (without corners) or can have one of more corners. A corner in an edge is a position on the edge where the direction of a tangent to the edge (where the tangent is parallel to the top surface of the plate) immediately next to the position on the edge is at least 10° different from the direction of a tangent to the edge (where the tangent is parallel to the top surface of the plate) immediately next to the other side of the position on the edge. It is preferred that plates have four corners of 90° each. That is, it is preferred that the plates have a rectilinear shape.

The top surface of the bottom plate can include multiple positionally distinguishable paths. Each path defines a pathway across the surface. The paths are distinguished from non-path areas on a surface by the presence of substrate oligomers on the paths. Non-paths do not have substrate oligomers. Generally, a path is continuous. The paths on a given surface can follow linear, curved, or a combination of linear and curved pathways. The paths on a given surface can have one or more changes in direction. The changes in direction can be by a curve, a corner, or a combination of a curve and a corner. Preferably, the paths follow a pathway traversing away from a first edge of the surface toward the opposite edge of the surface, changing direction to turn from the opposite edge back toward the first edge.

The multiple paths on the top surface of the bottom plate can be, and preferably are, parallel to adjacent paths. A full barcode of paths comprises a set of parallel paths where each path in the set comprises a different, detectably distinguishable substrate oligomer. Preferably, the paths in a full barcode of paths follow parallel paths that make turns together resulting in nested turns, with the outer path in the set of paths turning back immediately adjacent to the outer path and the inner path turning outside and around all of the other paths in the set. The effect is to produce multiple full barcodes of paths traversing the surface parallel to other full barcodes of paths traversing the surface. FIG. 5 shows an example of a preferred pattern of paths.

The width of paths are preferably as narrow as possible to allow production of the device and effective detection of a label or signal generated on the path. A narrower path allows more paths to fit in a given area on the surface of a plate, which in turn can allow more paths in a full barcode, more copies of full barcodes along the surface, or a combination of both. The paths can be, for example, about 5 μm wide to about 100 μm wide. Preferably, the paths are about 20 μm to about 60 μm wide, more preferably about 40 μm to about 50 μm wide, most preferably about 50 μm wide. Preferably, all of the paths on a given surface have substantially the same width.

The paths on a surface, such as paths in a barcode of paths, can have a pitch that defines the average spacing between the paths. The pitch of paths can be about 1.5 to about 3 times the width of the paths. Preferably, the pitch of paths can be 2 times the width of the paths. Thus, for example, the pitch of paths can be about 7.5 μm wide to about 300 μm wide. Preferably, the pitch of paths are about 40 μm to about 120 μm, more preferably about 80 μm to about 100 μm, most preferably about 100 μm. Preferably, all of the paths on a given surface or in a given barcode of paths have substantially the same pitch.

### D. Microchannel Molds

Microchannel molds out of any material an in any manner that can produced the required patterns of channels and/or wells are the desired scale. Generally, any materials and techniques used in the field of microfluidic devices can be used to produce the disclosed microchannel molds (use of the word mold is not intended to limit the manner of production or the form of the microchannel mold.

Useful materials include elastomers and thermoplastic materials. Materials useful for microfluidics can be categorized into three broad groups: inorganic, polymers, and paper. Beyond silicon and glass, inorganic materials extend over co-fired ceramics and vitroceramics. The second polymer-based category can be divided into two subcategories (i) thermoset materials, which are thermal or UV curable materials from a low viscosity precompound dispensed over a mold and (ii) thermoplastic materials, which are thermoformable materials amendable for rapid prototyping and manufacturing. Both polymer subcategories display rigid to elastomer mechanical properties, and through adaptable formulation and enriched chemical modification, offer a broad range of physicochemical surface properties. Paper microfluidics is generally based on a patterning approach, where devices drive liquid through capillary actions via wicking in a cellulose matrix. Because preferred forms of the disclosed devices and methods use pressure to flow solution through the microfluidics, and because of design advantages, polymer-based materials are preferred for the microfluidic components of the disclosed devices and methods.

Thermoset material include thermal and UV curable materials, such as polydimethylsiloxane (PDMS) and SU-8 photoresist, respectively. Thermoplastic materials include, for example, polycyclo-olefin (PCO), polycarbonate (PC), polytetrafluoroethylene (PTFE) and polystyrene (PS). Polycyclo-olefin offers high moldability and low water uptake. Polycarbonate has excellent material toughness properties while polytetrafluoroethylene and polyimide feature excellent chemical resistance, electrical, and thermal properties, respectively. Polystyrene is useful for cellular-based microfluidic systems. Elastomers, such as silicone elastomers, can be molded into microfluidic components, such as the microchannel molds. Such elastomers can be either or both thermoreactive (setting upon cooling) or curable (setting after curing reaction, baking, or both).

In some forms, the solid substrate of any of the disclosed devices, methods, and systems can be associated with a microfluidic component so to allow microfluidic based production of the disclosed devices and solid substrates or performance of microfluidic based assays. Microfluidic-based production and assays offer advantages such as reduced sample and reagent volumes, and shortened assay times. For example, under certain operational conditions, the surface binding assay kinetics are primarily determined by the analyte (protein) concentration and the analyte/antigen binding affinity, rather than by diffusion.

The term “microfluidic” as used herein refers to a component or system that has microfluidic features (e.g., channels and/or chambers) that are generally fabricated on the micron or sub-micron scale. For example, the typical channels or chambers have at least one cross-sectional dimension in the range of about 0.1 microns to about 1500 microns, more typically in the range of about 0.2 microns to about 1000 microns, still more typically in the range of about 0.4 microns to about 500 microns. Individual microfluidic features typically hold very small quantities of fluid, e.g., from about 10 nanoliters to about 5 milliliters, more typically from about 100 nanoliters to about 2 milliliters, still more typically from about 200 nanoliters to about 500 microliters, or yet more typically from about 500 nanoliters to about 200 microliters.

The microfluidic components can be included in an integrated device. As used herein, “integrated device” refers to a device having two (or more) components physically and operably joined together. For example, the bottom plate and the microchannel mold or the tope plate. The components may be (fully or partially) fabricated separate from each other and joined after their (full or partial) fabrication, or the integrated device may be fabricated including the distinct components in the integrated device. An integrated microfluidic array device includes an array component joined to a microfluidic component, wherein the microfluidic component and the array component are in operable association with each other such that an array substrate of the array component is in fluid communication with a microfluidic feature of the microfluidic component. A microfluidic component is a component that includes a microfluidic feature and is adapted to being in operable association with an array component. An array component is a component that includes a substrate and is adapted to being in operable association with a microfluidic component. For example the paths of substrate oligomers attached to a solid substrate with a microchannel mold that forms either channels or wells over the paths is a form of integrated microfluidic array device.

The microfluidic systems can also be provided in a modular form. “Modular” describes a system or device having multiple standardized components for use together, wherein one of multiple different examples of a type of component may be substituted for another of the same type of component to alter the function or capabilities of the system or device; in such a system or device, each of the standardized components being a “module.”

In microfluidic embodiments of the methods and systems herein disclosed, measurements of large panels of protein biomarkers within extremely small sample volumes and a much reduced background/biofouling are possible.

In the microfluidic embodiments of the methods and systems herein disclosed, the sensitivity of the assay can also be increased.

Additionally, the microfluidic methods and systems herein disclosed allow performance of both (i) mono step assays (where the labelled candidate molecules, the assay molecule(s), and the imaging agent are contacted in a single step) and (ii) multi-steps assays (where the solid substrate is sequentially exposed to the labelled candidate molecules, the assay molecule(s), and the imaging agent) in a reduced amount of time, with samples reduced in size and with a higher sensitivity when compared with corresponding microfluidic methods and system of the art and with other non-microfluidic methods and systems for molecule detection

An additional advantages associated with microfluidic methods and systems herein disclosed includes the possibility of performing in a microfluidic environment any assay that involves substrate-supported antibodies, which would not have survived microfluidic chip assembly with the use of previous techniques.

### E. Assay Molecules

Assay molecules are any compound or molecule to be used to assess the properties of candidate molecules, to be assessed for the effect of candidate molecules on it, or both. In preferred forms, assay molecules are target molecules for candidate molecules to bind to, react to, or react on. Most preferable, the assay molecules are target molecules for candidate molecules to bind to. This last form is extensively explored in the examples where the binding affinity of candidate molecules to a target protein are assessed using examples of the disclosed devices and methods.

Because the disclosed devices and methods constitute a broadly applicable platform and system for assessing any set of candidate molecules of any type for a variety of properties and activities in response to any assay molecule of interest, there is no a priori limit to which candidate molecules and which assay molecules can be used with the disclosed devices and methods (except that the property or interaction to be assessed must be capable of assessment using the disclosed devices and methods.

Preferred assay molecules are molecules, such as proteins, peptides, and epitopes, for which specific binding molecules are sought. The disclosed devices and methods are particularly useful for screening candidate molecules for specific and high affinity binding to such target molecules.

### F. Imaging Agents

Imaging agents are compounds or molecules that include or produce a detectable label. A label is a compound or molecule that emits or produces a detectable signal. In some forms, the imaging agent specifically binds to an assay molecule or a complex or product produced during the assay. In this way, the imaging agent associates the label with a location (e.g., path) on the solid substrate where the assay molecule binds or where a complex or product is produced during the assay. This localization of the label and signal from the label is a highly preferred feature of preferred forms of the disclosed devices, components, and methods. This feature enhances the multiplexing and miniaturization advantages of the disclosed devices and methods.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine) Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

The use of fluorescent dyes is generally preferred as they can be detected at very low amounts. Labelled locations on the solid substrate can be detected using, for example, a fluorimeter, the presence of a signal indicating an imaging agent at that location.

Although any imaging agent that can produce a detectable signal based on an interaction or reaction (or both) in a well of the disclosed devices, it is preferred that the label on the imaging agent be a direct label. As used herein, a direct label is a label that produces a signal constantly (i.e., at all times), after remote stimulation (such as a fluorescent signal produced upon excitation with light), or upon binding to the target molecule of the imaging molecule. The hallmark of a direct label is the lack of a need or requirement for a reaction or production of a distinct physical product by or from the imaging agent. A label produced by an enzymatic reaction in the assay is an example of a label that is not a direct label.

In some forms of the method, an interferent molecule can be added to wells for the assays. An interferent molecule is any molecule known to or suspected of interacting with, binding, and/or affecting the binding or activity of a molecule. For example, in some forms, interferent molecules can be known to or suspected of interacting with, binding, and/or affecting the binding or activity of a candidate molecule, an assay molecule, or both. For example, an interferent molecule may compete with the assay molecule for binding to the candidate molecules or may inhibit reaction of the assay molecule with the candidate molecules. In some forms of the method, the interferent molecule can be a competitive binding protein. Interferent molecules can be useful as controls or as assay components that can probe different aspects of the binding, kinetics, or activity of candidate molecules or assay molecules.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for producing the disclosed devices, the kit comprising a solid substrate having wells and substrate oligomers in paths on the solid substrate in the wells, labelled scaffold molecules, and reagents to couple candidate molecules to the scaffold. In some forms, the kit can comprises a solid substrate having wells and substrate oligomers in paths on the solid substrate, a microchannel mold adhered to the solid substrate, labelled scaffold molecules, reagents to couple candidate molecules to the scaffold, and a top plate to form wells on the solid substrate.

### H. Mixtures

Disclosed are mixtures formed by performing or preparing to perform the disclosed method. For example, disclosed are mixtures comprising substrate oligomers, labelled candidate molecules, and a solid substrate.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### I. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising the disclosed devices and a device for detecting imaging agents. Also contemplated are systems comprising the disclosed devices and a device for automatically performing steps of one or more of the disclosed assays. Also contemplated are systems for producing the disclosed devices.

### J. Data Structures and Computer Control

Disclosed are data structures used in, generated by, or generated from, the disclosed methods. Data structures generally are any form of data, information, and/or objects collected, organized, stored, and/or embodied in a composition or medium. Assay results stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### A. Actions Based on Identifications

The disclosed methods include the determination, identification, indication, correlation, diagnosis, prognosis, etc. (which can be referred to collectively as “identifications”) of subjects, diseases, conditions, states, etc. based on measurements, detections, comparisons, analyses, assays, screenings, etc. For example, identify the most promising of a set of candidate molecules for further testing. Such identifications are useful for many reasons. For example, and in particular, such identifications allow specific actions to be taken based on, and relevant to, the particular identification made.

Accordingly, also disclosed herein are methods comprising taking particular actions following and based on the disclosed identifications. For example, disclosed are methods comprising creating a record of an identification (in physical—such as paper, electronic, or other—form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others.

The disclosed measurements, detections, comparisons, analyses, assays, screenings, etc. can be used in other ways and for other purposes than those disclosed. Thus, the disclosed measurements, detections, comparisons, analyses, assays, screenings, etc. do not encompass all uses of such measurements, detections, comparisons, analyses, assays, screenings, etc.

### Methods

### A. Producing Devices

Disclosed are methods of producing the disclosed devices and solid substrates. While the disclosed devices and solid substrates can be produced in any manner and in combination that can result in devices and solid substrates as disclosed, it is preferred that the devices and solid substrates are produced using materials, modes, and techniques that produce some or all of the benefits of the disclosed devices and solid substrates: production that is efficient in time required, effort required, manipulations required, or a combination (generally meaning fewer or less of these). Features of the preferred forms of the methods of producing the devices and solid substrates illustrated by the example device production described in the examples. Generally, the size, number, pattern, order, nature, and/or relationship of the components of the devices and solid substrates, including the substrate oligomers, label oligomers, scaffold molecules, labelled candidate molecules, paths, bottom plates, microchannel molds, and/or top plates, can be chosen to support or enhance the efficiency of the production of the devices and solid substrates.

Useful features of the methods of producing the devices and solid substrates include materials and steps that allow all of the paths of attached substrate oligomers to be produced simultaneously, preferably with a single impetus for patterning the paths of the solid substrate. An example of such a useful feature is a microchannel mold adhered to the surface of the solid substrate, where the microchannel mold has channels for each path and ends of the channels that allow different substrate oligomers to be loaded in different paths but also allowing a single manifold with a single opening to be used to apply flow pressure to loaded substrate oligomers at the same time.

Another example of useful features of the methods of producing the devices and solid substrates is the use of sets of substrate oligomers and label oligomers that are bindingly distinguishable (i.e., are orthogonal). This allows a single solution of all of the labelled candidate molecules to be contacted with the solid substrate for attachment to their intended and distinct paths.

The methods of producing the disclosed devices and solid substrates includes methods of producing any of the intermediate forms of the devices and solid substrates. This includes, for example, methods of producing solid substrates, such as bottom plates, with material or components, such as polylysine, for attachment of the substrate oligomers to the solid substrate. Such a solid substrate could be packaged in kits or stored for later use, for example. As another example, methods of producing solid substrates, such as bottom plates, with material or components, such as microchannel molds, for patterned flow and attachment of the substrate oligomers to the solid substrate. Such a solid substrate could be packaged in kits or stored for later use, for example.

As another example, methods of producing solid substrates, such as bottom plates, with material or components, such as substrate oligomers, attached to the solid substrate in a useful pattern. Such a solid substrate could be packaged in kits or stored for later use, for example. Such a solid substrate is a preferred form of solid substrate for inclusion in kits, allowing users to attach candidate molecules of their choice to the solid substrate. Such a solid substrate can have the patterning component (e.g., microchannel mold) still adhered to the surface or such a solid substrate can be without the patterning component.

As another example, methods of producing solid substrates, such as bottom plates, with material or components, such as candidate molecules, attached to the solid substrate in a useful pattern. Such a solid substrate could be packaged in kits or stored for later use, for example. Such a solid substrate can have the patterning component (e.g., microchannel mold) still adhered to the surface or such a solid substrate can be without the patterning component.

As another example, methods of producing solid substrates, such as bottom plates, with material or components, such as top plates, attached to the solid substrate to form wells. Such a solid substrate could be packaged in kits or stored for later use, for example. Such a solid substrate is another preferred form of solid substrate for inclusion in kits, allowing users to just perform assays using a solid substrate having defined candidate molecules attached to the solid substrate. In some forms, sets of such solid substrates can be provided that include a library of candidate molecules for use in assays.

As another example, methods of producing solid substrates having wells and attached candidate molecules with material or components, such as imaging agents, present in the wells. Such a solid substrate could be packaged in kits or stored for later use, for example.

As another example, methods of producing solid substrates having wells, attached candidate molecules, with material or components, such as assay molecules, present in the wells. In some forms, this can be a method of producing an intermediate or final product of an assay method.

As another example, methods of producing solid substrates having wells, attached candidate molecules, assay molecules attached to the candidate molecules, with material or components, such as imaging agents, present in the wells. In some forms, this can be a method of producing an intermediate or final product of an assay method.

As another example, methods of producing solid substrates having wells, attached candidate molecules, assay molecules attached to the candidate molecules, and imaging agents attached to the assay molecules. In some forms, this can be a method of producing a final product of an assay method.

Disclosed are methods for producing devices embodying some or all of these useful features. In some forms, the methods involve contacting a solid substrate with a plurality of labelled candidate molecules, resulting in localization of each different labelled candidate molecule in a different path on the solid substrate, and, following or prior to contacting the solid substrate with the labelled candidate molecules, forming a plurality of test wells in the solid substrate, where one or more portions of each different path are in each well. In some forms of the method, the different labelled candidate molecules each comprise a different candidate molecule and a different label oligomer. In some forms of the method, the solid substrate comprises a plurality of the paths, where the paths are each positionally distinguishable and continuous. In some forms of the method, each of a plurality of different substrate oligomers is attached to a different one of the paths. In some forms of the method, each different label oligomer is complementary to a different one of the substrate oligomers. In some forms of the method, the label oligomers and the complementary substrate oligomers hybridize, where hybridization of a given label oligomer to the complementary substrate oligomer is bindingly distinguishable, which produces localization of each different labelled candidate molecule in a different one of the paths on the solid substrate.

In some forms of the method, each well can expose two or more different portions of each of the paths, where the two or more different portions of the paths are not continuous or contiguous in the well. In some forms of the method, each well exposes three different portions of each of the paths. In some forms of the method, the paths on the solid substrate change direction a plurality of times to form a serpentine pathway. In some forms of the method, one end of each path is proximal to a first side or edge of the solid substrate and the other end of each path is proximal to the side or edge of the solid substrate distal to the first side or edge of the solid substrate.

In some forms of the method, one or more of the paths constitutes a control path, wherein no labelled candidate molecule is localized in the control path. In some forms of the method, one or more of the control paths have a labelled control molecule localized in the control path, where the labelled control molecule is localized in the control path by, during the contacting step, contacting the solid substrate with the labelled control molecule. In some forms of the method, the labelled control molecule comprises a control molecule and a control label oligomer, where the control label oligomer is different from any of the label oligomers on the labelled candidate molecules localized on the solid substrate. In some forms of the method, the control label oligomer is complementary to one of the substrate oligomers, where the control label oligomer and the complementary substrate oligomer hybridize, resulting in localization of the control molecule in the path to which the complementary substrate oligomers is attached.

In some forms of the method, the paths can have a width of about 5 μm to about 100 μm. In some forms of the method, the paths can have a pitch of about 1.5 times to about 3 times the width of the paths. In some forms of the method, the paths can have a pitch of about 2 times the width of the paths. In some forms of the method, the width of the paths can be 50 μm. In some forms of the method, the paths can have a pitch of 100 μm.

In some forms of the method, each well can have an area of about 5 mm² to about 30 mm². In some forms of the method, each well can have an area of about 18 mm². In some forms of the method, the length of the shortest line that crosses all of the different paths in a wells can be about 450 μm to about 18 mm. In some forms of the method, the length of the shortest line that crosses all of the different paths in a well can be about 6 mm. In some forms of the method, the length of the shortest line that crosses the well can be about 150 μm to about 6 mm. In some forms of the method, the length of the shortest line that crosses the well can be about 3 mm.

In some forms of the method, the ratio of the length of the shortest line that crosses all of the different paths in a well and the length of the shortest line that crosses the well can be about 1 to about 5. In some forms of the method, the ratio of the length of the shortest line that crosses all of the different paths in a well and the length of the shortest line that crosses the well can be about 3. In some forms of the method, the solid substrate is rectangular. In some forms of the method, the solid substrate can comprise a glass slide or a plastic slide.

In some forms of the method, the method can further comprise coating the solid substrate with polylysine prior to attachment of the substrate oligomers to the solid substrate.

In some forms of the method, the solid substrate can comprise a bottom plate comprising a top surface, where the substrate oligomers are attached to the top surface of the bottom plate, where all of the paths are on the top surface of the bottom plate, and where the plurality of wells are formed by adhering a top plate to the top surface of the bottom plate. In some forms of the method, the top plate comprises perforations, where the wells comprise the surface of the bottom plate exposed by the perforations in the top plate. In some forms of the method, the top plate is a microchannel mold comprising the wells, where the wells are chambers over the surface of the bottom plate. In some forms of the method, the bottom plate is rectangular. In some forms of the method, the bottom plate is a glass slide or a plastic slide.

In some forms of the method, the method can further comprise coating the top plate with polylysine prior to attachment of the substrate oligomers to the solid substrate.

In some forms of the method, the method can further comprise, prior to contacting the solid substrate with the labelled candidate molecules and prior to forming the wells, adhering a microchannel mold onto the solid substrate, where the adhered microchannel mold forms a different continuous sealed channel above each path on the solid substrate; and flowing each different one of the substrate oligomers through a different formed channel and conjugating the substrate oligomers to the solid substrate.

In some forms of the method, contacting the solid substrate with the labelled candidate molecules can be accomplished by flowing the labelled candidate molecules through the formed channels. In some forms of the method, all of the labelled candidate molecules are flowed through each of the formed channels. In some forms of the method, each different one of the labelled candidate molecules is flowed through a different one of the formed channels. In some forms of the method, the method can further comprise, prior to forming the wells, removing the microchannel mold from the solid substrate. In some forms of the method, the microchannel mold can be fabricated from an elastomer. In some forms of the method, the wells can be formed prior to contacting the solid substrate with the labelled candidate molecules, where contacting the solid substrate with the labelled candidate molecules can be accomplished by adding all of the labelled candidate molecules to each of the wells. In some forms of the method, contacting the solid substrate with the labelled candidate molecules can be accomplished by adding all of the labelled candidate molecules to the solid substrate following removal of the microchannel mold and prior to forming the wells. In some forms of the method, contacting the solid substrate with the labelled candidate molecules can accomplished by adding all of the labelled candidate molecules to the solid substrate prior to forming the wells.

The disclosed methods can include any one of more of the disclosed steps of producing the disclosed devices and solid substrates can be performed. Generally, methods involving one or a subset of all of the steps needed to produced a given form of the disclosed devices and solid substrates can use as a starting material, a partially produced device or solid substrate. This would be the case, for example, where a user performed some steps of production, such as attaching candidate molecules, using a solid substrate that had previously had substrate oligomers attached.

The disclosed method can comprise one of more of the following steps:

(A) coating the solid substrate with a material, such as polylysine, that allows substrate oligomers to be attached to the solid substrate;

(B) adhering a microchannel mold onto the solid substrate;

(C) flowing each different one of the substrate oligomers through a different formed channel;
(C1) conjugating the substrate oligomers to the solid substrate;

(D) contacting a solid substrate with a plurality of labelled candidate molecules;
(D1) flowing the labelled candidate molecules through the formed channels;
(D2) adding all of the labelled candidate molecules to the solid substrate;

(E) removing the microchannel mold from the solid substrate;

(F) contacting the solid substrate with the labelled control molecule;

(G) forming a plurality of test wells in the solid substrate;
(G1) adhering a top plate to the top surface of the bottom plate;

(H) adding an assay molecule to each well of the solid substrate, optionally excepting a control well;

(I) adding an imaging agent to the solid substrate, such as in each well;

(J) detecting the imaging agent on a plurality of the paths in a plurality of the wells.

Generally:

step (A) should precede step (C);

step (B) should precede step (C) and preferably follows step (A);

step (C) should follow step (B) and precedes steps (D), (E), (F), and (I);

step (D) should follow step (3/C), preferably precedes steps (H) and (I), and preferably is performed at the same time as step (F);

step (E) should follow step (C) and preferably precedes step (G);

step (F) should follow step (C), preferably precede steps (H) and (I), and preferably is performed at the same time as step (D);

step (G) should precede step (H) and preferably follows step (E);

step (H) should follow step (G), preferably follows step (D), and preferably is performed at the same time as step (I);

step (I) should follow step (C), should precedes step (J), preferably follows steps (D), (G), and (H), and preferably is performed at the same time as step (I);

step (J) should follow steps (I), (H), (F), (E), and (D).

Every combination and order of steps that is consistent with these rules is specifically contemplated.

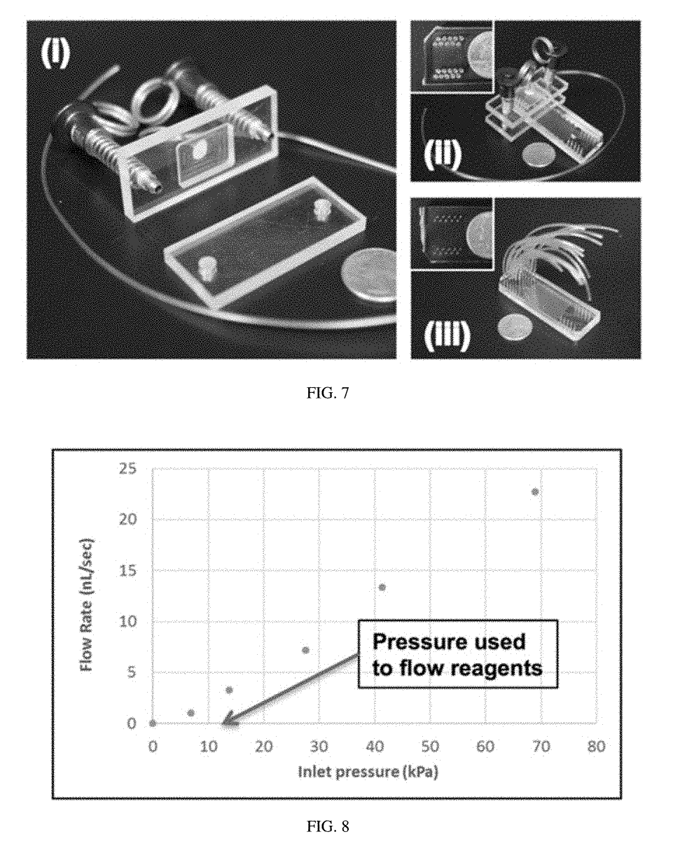

Steps (C), (D), and (F) can be performed by, for example, applying pressure at one end of the channels formed by the microchannel mold adhered to the surface of the solid substrate. The microchannel mold has channels for each path and ends of the channels that allow different substrate oligomers to be loaded in different paths. It is useful to be able to apply pressure from a single component that fits over and/or covers the one end of all of the channels (FIG. 7, panels (i) and (ii)), although multiple components can be used (FIG. 7, panel (iii)). Such a component can be referred to as a manifold. A single manifold with a single opening (technically a unifold, but still referred to herein as a manifold) is preferred (FIG. 7, panel (i)). Such a single manifold with a single opening can be used to apply flow pressure to loaded substrate oligomers at the same time. A single manifold having multiple openings can also be used. The multiple opening can each fit over and/or cover one end of one of more of the channels. Generally, the openings on the manifolds should, when mounted on the device or solid substrate, be in gaseous, fluid, or both gaseous and fluid communication with the end(s) covered by the manifold opening.

It is contemplated and should be understood that ancillary steps or sequential parts of a steps can be performed (and may be needed for certain forms of the methods and devices). For example, some of the steps of the methods should include washing steps, fixing steps, adhering steps, coupling steps, etc. Such steps generally follow from the nature of the reagents and components and the chemistries of the interactions involved. Those of skill in the art should be well aware of when, where, and how such steps will be useful or needed even if they are not detailed in all of the descriptions of the methods and method steps.

Some examples of these include a fixing or coupling step as part of or following, for example, one or a combination of steps (A), (C), (D), and (F). Other examples include incubations for certain times and/or under certain conditions as part of or following, for example, one or a combination of steps (A), (B), (C), (D), (E), (F), (G), (H), (I), and (J). Other examples include washes as part of or following, for example, one or a combination of steps (C), (D), (E), (F), (G), and (I).

Disclosed are methods for producing devices embodying some or all of these useful features. In some forms, the methods involve contacting a solid substrate with a plurality of labelled candidate molecules, resulting in localization of each different labelled candidate molecule in a different path on the solid substrate, and, following or prior to contacting the solid substrate with the labelled candidate molecules, forming a plurality of test wells in the solid substrate, where one or more portions of each different path are in each well. In some forms of the method, the different labelled candidate molecules each comprise a different labelled candidate molecule and a different label oligomer. In some forms of the method, the solid substrate comprises a plurality of the paths, where the paths are each positionally distinguishable and continuous. In some forms of the method, each of a plurality of different substrate oligomers is attached to a different one of the paths. In some forms of the method, each different label oligomer is complementary to a different one of the substrate oligomers. In some forms of the method, the label oligomers and the complementary substrate oligomers hybridize, where hybridization of a given label oligomer to the complementary substrate oligomer is bindingly distinguishable, which produces localization of each different labelled candidate molecule in a different one of the paths on the solid substrate.

In some forms of the method, each well can expose two or more different portions of each of the paths, where the two or more different portions of the paths are not continuous or contiguous in the well. In some forms of the method, each well exposes three different portions of each of the paths. In some forms of the method, the paths on the solid substrate change direction a plurality of times to form a serpentine pathway. In some forms of the method, one end of each path is proximal to a first side or edge of the solid substrate and the other end of each path is proximal to the side or edge of the solid substrate distal to the first side or edge of the solid substrate.

In some forms of the method, one or more of the paths constitutes a control path, wherein no labelled candidate molecule is localized in the control path. In some forms of the method, one or more of the control paths have a labelled control molecule localized in the control path, where the labelled control molecule is localized in the control path by, during the contacting step, contacting the solid substrate with the labelled control molecule. In some forms of the method, the labelled control molecule comprises a control molecule and a control label oligomer, where the control label oligomer is different from any of the label oligomers on the labelled candidate molecules localized on the solid substrate. In some forms of the method, the control label oligomer is complementary to one of the substrate oligomers, where the control label oligomer and the complementary substrate oligomer hybridize, resulting in localization of the control molecule in the path to which the complementary substrate oligomers is attached.

In some forms of the method, the paths can have a width of about 5 μm to about 100 μm. In some forms of the method, the paths can have a pitch of about 1.5 times to about 3 times the width of the paths. In some forms of the method, the paths can have a pitch of about 2 times the width of the paths. In some forms of the method, the width of the paths can be 50 μm. In some forms of the method, the paths can have a pitch of 100 μm.

In some forms of the method, each well can have an area of about 5 mm² to about 30 mm². In some forms of the method, each well can have an area of about 18 mm². In some forms of the method, the length of the shortest line that crosses all of the different paths in a wells can be about 450 μm to about 18 mm. In some forms of the method, the length of the shortest line that crosses all of the different paths in a well can be about 6 mm. In some forms of the method, the length of the shortest line that crosses the well can be about 150 μm to about 6 mm. In some forms of the method, the length of the shortest line that crosses the well can be about 3 mm.

In some forms of the method, the ratio of the length of the shortest line that crosses all of the different paths in a well and the length of the shortest line that crosses the well can be about 1 to about 5. In some forms of the method, the ratio of the length of the shortest line that crosses all of the different paths in a well and the length of the shortest line that crosses the well can be about 3. In some forms of the method, the solid substrate is rectangular. In some forms of the method, the solid substrate can comprise a glass slide or a plastic slide.

In some forms of the method, the method can further comprise coating the solid substrate with polylysine prior to attachment of the substrate oligomers to the solid substrate.

In some forms of the method, the solid substrate can comprise a bottom plate comprising a top surface, where the substrate oligomers are attached to the top surface of the bottom plate, where all of the paths are on the top surface of the bottom plate, and where the plurality of wells are formed by adhering a top plate to the top surface of the bottom plate. In some forms of the method, the top plate comprises perforations, where the wells comprise the surface of the bottom plate exposed by the perforations in the top plate. In some forms of the method, the top plate is a microchannel mold comprising the wells, where the wells are chambers over the surface of the bottom plate. In some forms of the method, the bottom plate is rectangular. In some forms of the method, the bottom plate is a glass slide or a plastic slide.

In some forms of the method, the method can further comprise coating the top plate with polylysine prior to attachment of the substrate oligomers to the solid substrate.

In some forms of the method, the method can further comprise, prior to contacting the solid substrate with the labelled candidate molecules and prior to forming the wells, adhering a microchannel mold onto the solid substrate, where the adhered microchannel mold forms a different continuous sealed channel above each path on the solid substrate; and flowing each different one of the substrate oligomers through a different formed channel and conjugating the substrate oligomers to the solid substrate.

In some forms of the method, contacting the solid substrate with the labelled candidate molecules can be accomplished by flowing the labelled candidate molecules through the formed channels. In some forms of the method, all of the labelled candidate molecules are flowed through each of the formed channels. In some forms of the method, each different one of the labelled candidate molecules is flowed through a different one of the formed channels. In some forms of the method, the method can further comprise, prior to forming the wells, removing the microchannel mold from the solid substrate. In some forms of the method, the microchannel mold can be fabricated from an elastomer. In some forms of the method, the wells can be formed prior to contacting the solid substrate with the labelled candidate molecules, where contacting the solid substrate with the labelled candidate molecules can be accomplished by adding all of the labelled candidate molecules to each of the wells. In some forms of the method, contacting the solid substrate with the labelled candidate molecules can be accomplished by adding all of the labelled candidate molecules to the solid substrate following removal of the microchannel mold and prior to forming the wells. In some forms of the method, contacting the solid substrate with the labelled candidate molecules can accomplished by adding all of the labelled candidate molecules to the solid substrate prior to forming the wells.

### B. Assaying Candidate Molecules

Disclosed are methods of using the devices with attached candidate molecules and formed wells to assay the candidate molecules. In some forms of the method, the method involves adding an assay molecule to each well of the solid substrate, optionally excepting a control well, adding an imaging agent to each well of the solid substrate, where the imaging agent binds to the assay molecule or to a product of the assay molecule, the candidate molecule, or the assay molecule and candidate molecule together, and detecting the imaging agent on a plurality of the paths in a plurality of the wells.

In some forms of the method, the imaging agent can be detected in each of the paths in each of the wells. In some forms of the method, the imaging agent produces a fluorescent signal. In some forms of the method, the imaging agent produces a fluorescent signal upon excitation without the need for binding to or reaction with another molecule. In some forms of the method, the imaging agent is detected with a fluorescence image scanner. In some forms of the method, the image scanner generates a digitized output, where the digitized output is plotted as curves appropriate for the type of assay for each of the candidate molecules. In some forms of the method, the digitized output is plotted as binding curves for each of the candidate molecules. In some forms of the method, the imaging agent is detected in the middle third of the paths in the wells.

In some forms of the method, a measured value of the detected imaging agent is produced by averaging the signals of the imaging agent detected at different points along the paths in the wells. In some forms of the method, a measured value of the detected imaging agent is produced for a given path in a given well by averaging the signals of the imaging agent detected at different points along the given path in the given well. In some forms of the method, a measured value of the detected imaging agent is produced for a given candidate molecule in a given well by averaging the signals of the imaging agent detected on the different paths for the given candidate molecule in the given well.

In some forms of the method, the imaging agent comprises a fluorophore-labelled binding molecule. In some forms of the method, the imaging agent comprises a first binding molecule that binds to the assay molecule and a fluorophore-labelled binding molecule that binds to the first binding molecule. In some forms of the method, the first binding molecule is a primary antibody. In some forms of the method, the fluorophore-labelled binding molecule is a fluorophore-labelled antibody. In some forms of the method, the imaging agent comprises a fluorophore-labelled antibody. In some forms of the method, the imaging agent comprises a primary antibody that binds to the assay molecule, and a fluorophore-labelled antibody that binds to the primary antibody.

In some forms of the method, a different concentration of the assay molecule is added to each well of the solid substrate.

In some forms of the method, an interferent molecule is added to each well of the solid substrate, where the interferent molecule competes with the assay molecule for binding to the candidate molecules or inhibits reaction of the assay molecule with the candidate molecules. In some forms of the method, the interferent molecule is a competitive binding protein. In some forms of the method, a different concentration of the interferent molecule is added to each of the wells of the solid substrate.

In some forms of the method, both the label oligomers and the substrate oligomers are ssDNA molecules.

In some forms of the method, the labelled candidate molecules each further comprise a scaffold molecule, where the label oligomer of each labelled candidate molecule is chemically bonded to the scaffold molecule of the labelled candidate molecule and the candidate molecule of each labelled candidate molecule is bound or chemically bonded to the scaffold molecule of the labelled candidate molecule. In some forms of the method, the candidate molecule of each labelled candidate molecule is bound to the scaffold molecule of the labelled candidate molecule via a biotin-streptavidin interaction, where the scaffold molecule comprises streptavidin and the biotin is coupled to the candidate molecule. In some forms of the method, the label oligomer of each labelled candidate molecule is bound to the scaffold molecule of the labelled candidate molecule via a cysteine residue on the scaffold molecule.

In some forms of the method, 1 to 10 copies of the same label oligomer are bonded to each scaffold molecule. In some forms of the method, 2 to 4 copies of the same label oligomer are bonded to each scaffold molecule. In some forms of the method, 4 copies of the same label oligomer are bonded to each scaffold molecule. In some forms of the method, the label oligomers are modified via succinimide chemistry to have a 5′-aminated oligonucleotide. In some forms of the method, a hydrazide moiety is introduced to the candidate molecules via reaction with an amino group, where a hydrazine bond forms between the hydrazide moiety of the candidate molecules and the 5′-aminated oligonucleotide of the label oligomers.

In some forms of the method, the solid substrate comprises 10 paths to 30 paths. In some forms of the method, the solid substrate comprises 15 paths to 25 paths. In some forms of the method, the solid substrate comprises 20 paths. In some forms of the method, the solid substrate comprises 10 different candidate molecules to 30 different candidate molecules. In some forms of the method, the solid substrate comprises 15 different candidate molecules to 25 different candidate molecules. In some forms of the method, the solid substrate comprises 20 different candidate molecules. In some forms of the method, the solid substrate comprises 10 different labelled candidate molecules to 30 different labelled candidate molecules. In some forms of the method, the solid substrate comprises 15 different labelled candidate molecules to 25 different labelled candidate molecules. In some forms of the method, the solid substrate comprises 20 different labelled candidate molecules.

In some forms, detection can be carried either via fluorescent based readouts, in which the imaging agent is labelled with flurophore which includes but not exhaustively small molecular dyes, protein chromophores, quantum dots, and gold nanoparticles In particular, in some forms, in any of the methods and systems herein disclosed, detection can be carried out on gold nanoparticle-labelled secondary detection systems in which a common photographic development solution can amplify the gold nanoparticles as further described below. Also, if the readout comes from dark field scattering of gold particles, single molecule digital proteomics is enabled. Additional techniques are identifiable by a skilled person upon reading of the present disclosure and will not be further discussed in details.

The terms “label” and “labelled molecule” as used herein as a component of a complex or molecule refer to a molecule capable of detection, including but not limited to radioactive isotopes, fluorophores, chemiluminescent dyes, chromophores, enzymes, enzyme substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence the wording and “labeling signal” as used herein indicates the signal emitted from the label that allows detection of the label, including but not limited to radioactivity, fluorescence, chemolumiescence, production of a compound in outcome of an enzymatic reaction and the likes. In particular gold nanoparticles can be used in a sandwich style detection assay, in which the detection complex is linked to a gold nanoparticle. This is most relevant in detecting small molecules like proteins, peptides, etc., as detecting cells can be simply carried out using traditional microscopy techniques.

The term “monoparameter assay” as used herein refers to an analysis performed to determine the presence, absence, or quantity of one target. The term “multiparameter assay” refers to an analysis performed to determine the presence, absence, or quantity of a plurality of targets. The term “multiplex” or “multiplexed” assays refers to an assay in which multiple assays reactions, e.g., simultaneous assays of multiple analytes, are carried out in a single reaction chamber and/or analyzed in a single separation and detection format.

The term “hit” refers to a test compound that shows desired properties in an assay. The term “test compound” refers to a chemical to be tested by one or more screening method(s) as a putative modulator. A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar and 10 micromolar. Test compound controls can include the measurement of a signal in the absence of the test compound or comparison to a compound known to modulate the target.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist.”

The term “inhibit” means to reduce or decrease in activity or expression. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The disclosed devices and methods can be further understood through the following numbered paragraphs.
1. A method comprising:
   contacting a solid substrate with a plurality of labelled candidate molecules,
   wherein the different labelled candidate molecules each comprise a different candidate molecule and a different label oligomer,
   wherein the solid substrate comprises a plurality of positionally distinguishable, continuous paths, wherein each of a plurality of different substrate oligomers is attached to a different one of the paths, wherein each different label oligomer is complementary to a different one of the substrate oligomers,
   wherein the label oligomers and the complementary substrate oligomers hybridize, wherein hybridization of a given label oligomer to the complementary substrate oligomer is bindingly distinguishable, wherein the hybridization results in localization of each different candidate molecule in each of the different paths; and
   following or prior to contacting the solid substrate with the labelled candidate molecules, forming a plurality of test wells in the solid substrate, wherein one or more portions of each different path are in each well.
2. The method of paragraph 1, wherein each well exposes two or more different portions of each of the paths, wherein the two or more different portions of the paths are not continuous or contiguous in the well.
3. The method of paragraph 2, wherein each well exposes three different portions of each of the paths.
4. The method of any one of paragraphs 1-3, wherein the paths on the solid substrate change direction a plurality of times to form a serpentine pathway.
5. The method of any one of paragraphs 1-4, wherein one end of each path is proximal to a first side or edge of the solid substrate and the other end of each path is proximal to the side or edge of the solid substrate distal to the first side or edge of the solid substrate.
6. The method of any one of paragraphs 1-5, wherein one or more of the paths constitutes a control path, wherein no candidate molecule is localized in the control path.
7. The method of paragraph 6, wherein one or more of the control paths have a labelled control molecule localized in the control path, wherein the labelled control molecule is localized in the control path by, during the contacting step, contacting the solid substrate with the labelled control molecule,
   wherein the labelled control molecule comprises a control molecule and a control label oligomer, wherein the control label oligomer is different from any of the label oligomers on the labelled candidate molecules localized on the solid substrate,
   wherein the control label oligomer is complementary to one of the substrate oligomers, wherein the control label oligomer and the complementary substrate oligomer hybridize, resulting in localization of the control molecule in the path to which the complementary substrate oligomers is attached.
8. The method of any one of paragraphs 1-7, wherein the paths have a width of about 5 μm to about 100 μm.
9. The method of paragraph 8, wherein the paths have a pitch of about 1.5 times to about 3 times the width of the paths.
10. The method of paragraph 9, wherein the paths have a pitch of about 2 times the width of the paths.
11. The method of any one of paragraphs 8-10, wherein the width of the paths is 50
12. The method of any one of paragraphs 9-11, wherein the paths have a pitch of 100 μm.
13. The method of any one of paragraphs 1-12, wherein each well has an area of about 5 mm² to about 30 mm².
14. The method of any on of paragraphs 1-13, wherein each well has an area of about 18 mm².
15. The method of any one of paragraphs 1-14, wherein the length of the shortest line that crosses all of the different paths in a well is about 450 μm to about 18 mm
16. The method of any one of paragraphs 1-15, wherein the length of the shortest line that crosses all of the different paths in a well is about 6 mm.
17. The method of any one of paragraphs 1-16, wherein the length of the shortest line that crosses the well is about 150 μm to about 6 mm.
18. The method of any one of paragraphs 1-17, wherein the length of the shortest line that crosses the well is about 3 mm.
19. The method of any one of paragraphs 1-18, wherein the ratio of the length of the shortest line that crosses all of the different paths in a well and the length of the shortest line that crosses the well is about 1 to about 5.
20. The method of any one of paragraphs 1-19, wherein the ratio of the length of the shortest line that crosses all of the different paths in a well and the length of the shortest line that crosses the well is about 3.
21. The method of any one of paragraphs 1-20, wherein the solid substrate is rectangular.
22. The method of any one of paragraphs 1-21, wherein the solid substrate comprises a glass slide or a plastic slide.
23. The method of any one of paragraphs 1-22 further comprising, prior to attachment of the substrate oligomers to the solid substrate, the solid substrate is coated with polylysine.
24. The method of any one of paragraphs 1-23, wherein the solid substrate comprises a bottom plate comprising a top surface, wherein the substrate oligomers are attached to the top surface of the bottom plate, wherein all of the paths are on the top surface of the bottom plate, wherein the plurality of wells are formed by adhering a top plate to the top surface of the bottom plate.
25. The method of paragraph 24, wherein the top plate comprises perforations, wherein the wells comprise the surface of the bottom plate exposed by the perforations in the top plate.
26. The method of paragraph 24, wherein the top plate is a microchannel mold comprising the wells, wherein the wells are chambers over the surface of the bottom plate.
27. The method of any one of paragraphs 24-26, wherein the bottom plate is rectangular.
28. The method of any one of paragraphs 24-27, wherein the bottom plate is a glass slide or a plastic slide.
29. The method of any one of paragraphs 24-28 further comprising, prior to attachment of the substrate oligomers to the solid substrate, the top plate is coated with polylysine.
30. The method of any one of paragraphs 1-29 further comprising, prior to contacting the solid substrate with the labelled candidate molecules and prior to forming the wells,
   adhering a microchannel mold onto the solid substrate, wherein the adhered microchannel mold forms a different continuous sealed channel above each path on the solid substrate; and
   flowing each different one of the substrate oligomers through a different formed channel and conjugating the substrate oligomers to the solid substrate.
31. The method of paragraph 30, wherein contacting the solid substrate with the labelled candidate molecules is accomplished by flowing the labelled candidate molecules through the formed channels.
32. The method of paragraph 31, wherein all of the labelled candidate molecules are flowed through each of the formed channels.
33. The method of paragraph 31, wherein each different one of the labelled candidate molecules is flowed through a different one of the formed channels.
34. The method of any one of paragraphs 30-33 further comprising, prior to forming the wells, removing the microchannel mold from the solid substrate.
35. The method of any one of paragraphs 30-34, wherein the microchannel mold is fabricated from an elastomer.
36. The method of any one of paragraphs 1-30, wherein the wells are formed prior to contacting the solid substrate with the labelled candidate molecules, wherein contacting the solid substrate with the labelled candidate molecules is accomplished by adding all of the labelled candidate molecules to each of the wells.
37. The method of any one of paragraph 30, wherein contacting the solid substrate with the labelled candidate molecules is accomplished by adding all of the labelled candidate molecules to the solid substrate following removal of the microchannel mold and prior to forming the wells.
38. The method of any one of paragraphs 1-29, wherein contacting the solid substrate with the labelled candidate molecules is accomplished by adding all of the labelled candidate molecules to the solid substrate prior to forming the wells.
39. The method of any one of paragraphs 1-38 further comprising, following contacting the solid substrate with the labelled candidate molecules and to forming the wells:
   adding an assay molecule to each well of the solid substrate, optionally excepting a control well,
   adding an imaging agent to each well of the solid substrate, wherein the imaging agent binds to the assay molecule or to a product of the assay molecule, the candidate molecule, or the assay molecule and candidate molecule together,
   detecting the imaging agent on a plurality of paths in each of a plurality of the wells.
40. The method of paragraph 39, wherein the imaging agent is detected in each of the paths in each of the wells.
41. The method of paragraph 39 or 40, wherein the imaging agent produces a fluorescent signal.
42. The method of paragraph 41, wherein the imaging agent produces a fluorescent signal upon excitation without the need for binding to or reaction with another molecule.
43. The method of any one of paragraphs 39-42, wherein the imaging agent is detected with a fluorescence image scanner.
44. The method of paragraph 43, wherein the image scanner generates a digitized output, wherein the digitized output is plotted as curves appropriate for the type of assay for each of the candidate molecules.
45. The method of paragraph 44, wherein the digitized output is plotted as binding curves for each of the candidate molecules.
46. The method of any one paragraphs 39-45, wherein the imaging agent is detected in the middle third of the paths in the wells.
47. The method of any one paragraphs 39-46, wherein a measured value of the detected imaging agent is produced by averaging the signals of the imaging agent detected at different points along the paths in the wells.
48. The method of paragraph 47, wherein a measured value of the detected imaging agent is produced for a given path in a given well by averaging the signals of the imaging agent detected at different points along the given path in the given well.
49. The method of paragraph 47 or 48, wherein a measured value of the detected imaging agent is produced for a given candidate molecule in a given well by averaging the signals of the imaging agent detected on the different paths for the given candidate molecule in the given well.
50. The method of any one of paragraphs 39-49, wherein the imaging agent comprises a fluorophore-labelled binding molecule.
51. The method of any one of paragraphs 39-49, wherein the imaging agent comprises a first binding molecule that binds to the assay molecule and a fluorophore-labelled binding molecule that binds to the first binding molecule.
52. The method of paragraph 51, wherein the first binding molecule is a primary antibody.
53. The method of paragraph 51 or 52, wherein the fluorophore-labelled binding molecule is a fluorophore-labelled antibody.
54. The method of any one of paragraphs 39-49, wherein the imaging agent comprises a fluorophore-labelled antibody.
55. The method of paragraph 54, wherein the imaging agent comprises a primary antibody that binds to the assay molecule, and a fluorophore-labelled antibody that binds to the primary antibody.
56. The method of any one of paragraphs 39-55, wherein a different concentration of the assay molecule is added to each well of the solid substrate.
57. The method of any one of paragraphs 39-56, wherein an interferent molecule is added to each well of the solid substrate, wherein the interferent molecule competes with the assay molecule for binding to the candidate molecules or inhibits reaction of the assay molecule with the candidate molecules.
58. The method of paragraph 57, wherein the interferent molecule is a competitive binding protein.
59. The method of paragraph 57 or 58, wherein a different concentration of the interferent molecule is added to each of the wells of the solid substrate.
60. The method of any one of paragraphs 1-59, wherein both the label oligomers and the substrate oligomers are ssDNA molecules.
61. The method of any one of paragraphs 1-60, wherein the labelled candidate molecules each further comprise a scaffold molecule, wherein the label oligomer of each labelled candidate molecule is chemically bonded to the scaffold molecule of the labelled candidate molecule and the candidate molecule of each labelled candidate molecule is bound or chemically bonded to the scaffold molecule of the labelled candidate molecule.
62. The method of paragraph 61, wherein the candidate molecule of each labelled candidate molecule is bound to the scaffold molecule of the labelled candidate molecule via a biotin-streptavidin interaction, wherein the scaffold molecule comprises streptavidin and the biotin is coupled to the candidate molecule.
63. The method of paragraph 61 or 62, wherein the label oligomer of each labelled candidate molecule is bound to the scaffold molecule of the labelled candidate molecule via a cysteine residue on the scaffold molecule.
64. The method of any one of paragraphs 61-63, wherein 1 to 10 copies of the same label oligomer are bonded to each scaffold molecule.
65. The method of any one of paragraphs 61-64, wherein 2 to 4 copies of the same label oligomer are bonded to each scaffold molecule.
66. The method of any one of paragraphs 61-65, wherein 4 copies of the same label oligomer are bonded to each scaffold molecule.
67. The method of any one of paragraphs 1-60, wherein the label oligomers are modified via succinimide chemistry to have a 5′-aminated oligonucleotide.
68. The method of paragraph 67, wherein a hydrazide moiety is introduced to the candidate molecules via reaction with an amino group, wherein a hydrazine bond forms between the hydrazide moiety of the candidate molecules and the 5′-aminated oligonucleotide of the label oligomers.
69. The method of any one of paragraphs 1-68, wherein the solid substrate comprises 10 paths to 30 paths.
70. The method of any one of paragraphs 1-69, wherein the solid substrate comprises 15 paths to 25 paths.
71. The method of any one of paragraphs 1-70, wherein the solid substrate comprises 20 paths.
72. The method of any one of paragraphs 1-71, wherein the solid substrate comprises 10 different candidate molecules to 30 different candidate molecules.
73. The method of any one of paragraphs 1-72, wherein the solid substrate comprises 15 different candidate molecules to 25 different candidate molecules.
74. The method of any one of paragraphs 1-73, wherein the solid substrate comprises 20 different candidate molecules.
75. A device for simultaneously testing a plurality of candidate molecules, the device comprising a solid substrate made by the method of any one of paragraphs 1-38.

### Examples

### Materials

### Barcode Microfabrication and Validation

Chrome masks of the custom barcode design were purchased from University of California, Los Angeles, Nanoelectronics Research Facility, and a Karl Süss MA/BA6 mask aligner (SÜSS MicroTec AG) was used for UV exposure. Silicon wafers (Wafernet Inc.), SU8-2025, and SU8 developer (Microchem Corp) were used for the barcode mold fabrication. Anhydrous dimethylsulfoxide (DMSO), sodium dodecyl sulfate (SDS), and bis(sulfosuccinimidyl)suberate) (BS3) used in barcode fabrication were purchased from American Type Culture Collection (ATCC), Sigma Aldrich, and ThermoFischer Scientific respectively. The Sylgard 184 elastomer, and poly-L-lysine coated glass slides used in DNA barcode microfabrication were purchased from Dow Corning and ThermoFischer Scientific respectively. The poly-L-lysine (PLL) solution (0.1% (^{w}/_{w}) used for barcode fabrication was purchased from Sigma Aldrich. All ssDNA used for barcode fabrication and barcode validations were purchased from either Bioneer Inc. or IDT Inc.

### Protein Expression, Purification, and Refolding

The Bacto Tryptone (Tryptone) and Bacto yeast (yeast) for the preparation of LB broth media were purchased from Becton, Dickinson, and Company. The ampicillin sodium salt, chloramphenicol, and isopropyl β-D-1-thiogalactopyranoside (1,6-IPTG, dioxane free) used for protein expression from E. coli were purchased from Sigma Aldrich. The one-shot B21(D3) E. coli cells and PQE80 vector (His₆-tagged human KRas Isoform 4B (residues 1-169)) used for expression of KRas protein were purchased from Life Technologies and Qiagen respectively. Lysozyme (L6876), DNAse I (10104159001), and RNAse A (R6513-10MG) used for lysing cells containing cysteine-modified streptavidin (SAC) were purchased from Sigma-Aldrich. Cells containing KRas protein were lysed using a constant pressure cell disruptor (Constant Systems Ltd., Scotland, UK). Surfactants Triton X-100 and polysorbate 20 (Tween20) were purchased from Sigma Aldrich. The 20× phosphate buffered saline with 0.05% Tween 20 (PBST) and phosphate buffered saline (PBS) used for protein purification and immunofluorescent assays (IFAs) were purchased from Cell Signaling Technology and Corning respectively. The sodium bicarbonate (NaHCO₃), ammonium acetate (NH₄OAc), sodium acetate (NaOAc), sodium chloride (NaCl), imidazole, tris(hydroxymethyl)aminomethane (Tris), tris(hydroxymethyl)aminomethane hydrochloride salt (Tris.HCl), guanidinium chloride (Guan.HCl), magnesium chloride pentahydrate (MgCl₂.5H₂O), and beta-mercaptoethanol (βME) used in protein purification and IFA assays were purchased from Sigma Aldrich. The 2-imminobiotin agarose resin, Superdex 75 (10/300) increase column, and Ni-NTA superflow cartridge used for fast protein liquid chromatography (FPLC) purification were purchased from Sigma Aldrich, GE Healthcare Life Sciences, and Qiagen respectively. The Amicon Ultra-15 and Ultra-4 centrifugal filters used to concentrate protein samples were purchased from EMD Millipore.

### SAC-DNA Conjugation and DESL Set Validation

The tris-(2-carboxyethyl)phosphine hydrochloride (TCEP), anhydrous N,N-dimethylformamide (DMF), N-succimidly-4-formyl benzaldehyde (S-4FB) and maleimide 6-hydrazino-nicotinamide (MHPH) used for the conjugation of ssDNA to cysteine-modified streptavidin (SAC) were purchased from Sigma Aldrich and Solulink. The biotin-A₂₀-Cy3 (Biotin*) probe used to test the biotin binding ability of the DESL set and used as a biotinylated blank for IFA assays was purchased from IDT Inc. The complementary ssDNA′ used for conjugation to SAC were purchased from Bioneer Inc.

### In Situ Library Screen and Hit Bead Sequencing

The mouse anti-biotin-alkaline phosphatase conjugated antibody (ab) (#A6561), goat anti-rabbit-alkaline phosphatase conjugated ab (#A8025), rabbit anti-Ras ab (CST #3965), used for the combined anti screen/pre-clear and the subsequent product/target screens were purchased from Sigma Aldrich and Cell Signaling Technology respectively. The combined 5-bromo-4-chloro-3-indoyl phosphate (BCIP)/nitro blue tetrazolium (NBT) (#53771) used to develop hits during the library screens was purchased from Promega. The concentrated hydrochloric acid used to quench the BCIP/NBT development was purchased from Sigma Aldrich. Sequencing of bead hits occurred via Edman degradation sequencing on a Procise Protein Sequencer (Applied Biosystems, California).

### Peptide Synthesis and Purification

Fmoc-protected amino acids were purchased from Anaspec, AAPTec, Bachem, ChemPep, and Sigma-Aldrich. Biotin NovaTag™ resin was obtained from EMD Chemicals, Inc. and used for the synthesis of biotinylated peptides and epitopes used for the screens using standard Fmoc/^{t}Bu coupling and cleavage protocols. The peptide one-bead-one compound (OBOC) library was prepared on Tentagel Resin purchased from RAPP Polymere. The Fmoc-protected propionic acid polyethylene glycol (PEW linkers were purchased from ChemPep Inc. The L-ascorbic acid and copper (I) iodide (CuI) used for click reactions were purchased from Sigma Aldrich. The N-methyl pyrrolidine (NMP), 1-[Bis(dimethylamino)methylene]-1H-1,2,3-triazolo[4,5-b]pyridinium 3-oxid hexafluorophosphate (HATU), and N,N′-diisopropylethylamine (DIPEA) used in peptide synthesis were bought from EMD Chemicals, Inc., ChemPep, and Sigma-Aldrich respectively. Piperidine, trifluoroacetic acid (TFA), and triethylsilane (TESH) were purchased from Sigma-Aldrich. The diethyl ether used to precipitate crude peptide was purchased from JT Baker. The Omnisolv grade acetonitrile (MeCN) used for peptide purification was purchased from EMD Millipore. Unless otherwise stated, peptide preparation was performed using a Titan 357 Automatic Peptide Synthesizer (AAPPTec, Louisville, Ky.) or a Liberty 1 Automated Peptide Synthesizer (CEM, North Carolina). Mass analysis was performed using a Voyager De-Pro matrix assisted laser desorption ionization time-of-flight mass spectrometer (MALDI-TOF MS) (Applied Biosystems, California). The crude peptides were dissolved in either DMSO (Sigma Aldrich) or (1:1) MeCN/doubly distilled water (MQ H₂O) w/0.1% TFA before purification by a gradient of 0% to 50% acetonitrile in MQ H₂O with 0.01% (^{v}/ᵥ) TFA using a RP-HPLC (Beckman Coulter System Gold 126 Solvent Module and 168 Detector) using a C18 reversed phase semi-preparative column (Phenomenex Luna 10 μm, 250×10 mm). The concentration of peptides and epitopes was determined using a Nanodrop 2000 Spectrophotomer (ThermoFischer Scientific Inc., Massachusetts).

### B-RAP Immunofluorescent Assays and Multi-Well Enzyme-Linked Immunosorbent Assays

The Bovine Serum Albumin (BSA, Biotin free A1933-25G) used in the IFAs and multi-well enzyme-linked Immunosorbent assays (ELISAs) was purchased from Sigma-Aldrich. The non-fat dry milk powder used in the enzyme-linked Immunosorbent assays (ELISAs) was purchased from Best Value. The rabbit anti-Ras (CST #3965), Goat anti-rabbit IgG HRP-linked (CST #7074), goat anti-rabbit HRP-linked (CST #7074), and goat anti-rabbit-Alexafluor 647 conjugated (ab150079) were purchased from Cell Signaling Technologies and Abcam respectively. The ELISAs were run on either 96-well clear Pierce Neutravidin Plates (#15129) or Pierce Neutravidin Coated Plates (#15127) purchased from ThermoFischer Scientific. The TMB Microwell Peroxidase Substrate System (#50-76-00) that was used to develop ELISAs was purchased from KPL. The sulfuric acid (H₂SO_{4(aq)} used to quench the enzymatic amplification reaction in the ELISAs was purchased from JT Baker. The 96-well ELISA plates were read using a Flexstation 3 plate reader (Molecular Devices LLC, Sunnyvale, Calif.). All barcode slides were scanned using an Axon GenePix 4400A (Molecular Devices LLC, Sunnyvale, Calif.).

### Measuring the Functional Effect of the Allosteric Ligands on KRas Protein GTPase Activity

The intrinsic GTPase activity of WT KRas protein was measured using the GTPase-Glo Assay Kit (#V7681) from Promega Corporation (Madison, Wis.) on opaque white 96-well plates (#6005290) from Perkin Elmer Life Sciences (Waltham, Mass.). Luminescence was recorded on the Flexstation 3 plate reader used for multi-well ELISAs.

### Methods

### Preparation of the Barcode Rapid Assay Platform

DNA flow-patterned barcode chips, biotinylated peptides, and SAC-DNA were all used to assemble a miniaturized barcode of candidate PCCs for testing in a surface Immunofluorescent assay (IFA). Microfluidic flow patterning of 50 μm wide, 100 μm pitch ssDNA barcodes starts with adhering a polydimethylsiloxane (PDMS) microchannel mold onto a poly-L-lysine (PLL) coated glass microscope slide (FIGS. 7 and 8). Reagents were flowed through the microchannels using a “pins-and-tubing-free” system that greatly simplified the preparation of barcoded microchips relative to the previous protocols (FIG. 5; Table 3) (Yu et al., Annu. Rev. Anal. Chem. 2014, 7 (1), 275-295; Wei et al., Cancer Cell 2016, 29 (4), 563-573). The 50 μm barcode chip layout shown in FIG. 5A encompasses the entire length of a 3″ microscope slide. Input and outputs of the serpentine microchannels are at the right and left sides (FIG. 5A).

TABLE 3
A comparison of the various steps associated with the Solution
Loading Method (reported here) and the previously reported
Tubing Method for preparing microfluidic flow-patterned
ssDNA barcodes. By using the solution loading method, 20-25
devices are handled within one hour as opposed to 8-10 devices
with individual tubing for each amine DNA.

| | Solution Loading Method | Tubing Method |
|---|---|---|
| DNA Patterning step | (new) | (old) |
| Polylysine loading | 12 min | >24 min |
| Polylysine flow setting | 10 min | 20 min |
| DNA tubing preparation | n.a. | 30 min |
| DNA loading | 22 min | 50 min |

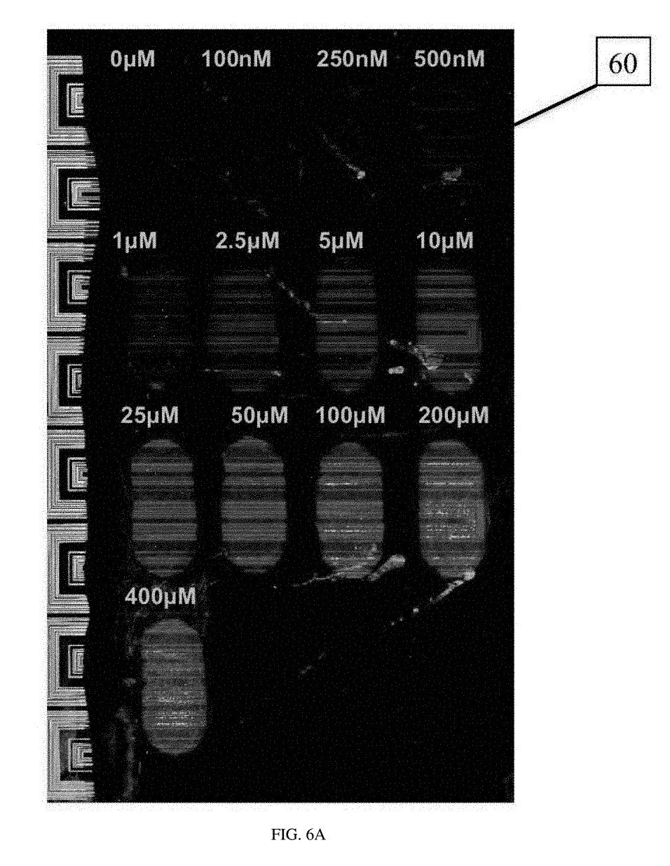

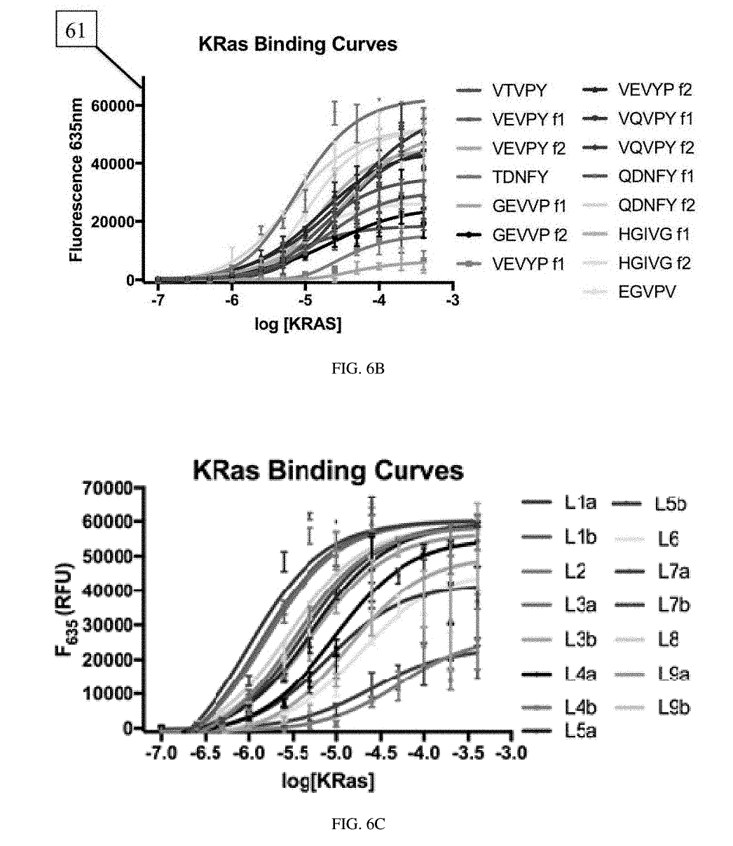

The PDMS mold was patterned with microwells at each microchannel inlet (FIG. 7, panels (i) and (ii)). Reagents (3-5 μL) are micropipetted into the wells, and two machined acrylic plates are clamped across the top and bottom of the inlet region. The top acrylic plate contains a cavity that encompasses all of the inlet microwells. This cavity is pressurized to fill the microchannels in about 20 minutes (FIGS. 5 and 7). The increased pressure tolerance of the design can enable the use of microchannels of widths as small as 10 μm. Initially 3 μL of poly-L-lysine (0.1% (^{w}/_{w}) in H₂O) is flow patterned and dried overnight before flowing 5 μL of 300 μM of each ssDNA (Table 4) with 2 mM bis(sulfosuccinimidyl)suberate) (BS3) crosslinker. Approximately 20 to 25 DNA barcoded chips may be prepared in parallel. The bottom edge of the barcode is used to validate the coverage density and uniformity of the molecular patterns using fluorophore-labelled complementary ssDNA (FIGS. 5 and 6A). Measuring the barcode quality across the entire barcode region (about 3.8 cm) reveals that all of the barcode lanes meet the minimum F₅₃₂ requirement for good levels of ssDNA patterning. Once validated, the barcoded slides may be vacuum-sealed for up to six months storage before use.

TABLE 4
Table of ssDNA sequences used for SAC-DNA conju-
gation and DNA barcode patterning. The sequences
with a DNA i.d. with an apostrophe (') are used
for conjugation to SAC protein.

| | SEQ | |
|---|---|---|
| DNA | ID | |
| i.d. | NO | Sequence |
| B | 1 | 5′-NH2-C6-AAA AAA AAA AAA AGC CTC ATT |
| | | GAA TCA TGC CTA-3′ |
| B' | 2 | 5′-NH2-C6-AAA AAA AAA ATA GGC ATG ATT |
| | | CAA TGA GGC-3′ |
| C | 3 | 5′-NH2-C6-AAA AAA AAA AAA AGC ACT CGT |
| | | CTA CTA TCG CTA-3′ |
| C' | 4 | 5′-NH2-C6-AAA AAA AAA ATA GCG ATA GTA |
| | | GAC GAG TGC-3′ |
| D | 5 | 5′-NH2-C6-AAA AAA AAA AAA GGT CGA GAT |
| | | GTC AGA GTA-3′ |
| D' | 6 | 5′-NH2-C6-AAA AAA AAA ATA CTC TGA CAT |
| | | CTC GAC CAT-3′ |
| E | 7 | 5′-NH2-C6-AAA AAA AAA AAA AAT GTG AAG |
| | | TGG CAG TAT CTA-3′ |
| E' | 8 | 5′-NH2-C6-AAA AAA AAA ATA GAT ACT GCC |
| | | ACT TCA CAT-3′ |
| F | 9 | 5′-NH2-C6-AAA AAA AAA AAA AAT CAG GTA |
| | | AGG TTC ACG GTA-3′ |
| F' | 10 | 5′-NH2-C6-AAA AAA AAA ATA CCG TGA ACC |
| | | TTA CCT GAT-3′ |
| G | 11 | 5′-NH2-C6-AAA AAA AAA AAA AGA GTA GCC |
| | | TTC CCG AGC ATT-3′ |
| G' | 12 | 5′-NH2-C6-AAA AAA AAA AAA TGC TCG GGA |
| | | AGG CTA CTC-3′ |
| H | 13 | 5′-NH2-C6-AAA AAA AAA AAA AAT TGA CCA |
| | | AAC TGC GGT GCG-3′ |
| H' | 14 | 5′-NH2-C6-AAA AAA AAA ACG CAC CGC AGT |
| | | TTG GTC AAT-3′ |
| I | 15 | 5′-NH2-C6-AAA AAA AAA AAA ATG CCC TAT |
| | | TGT TGC GTC GGA-3′ |
| I' | 16 | 5′-NH2-C6-AAA AAA AAA ATC CGA CGC AAC |
| | | AAT AGG GCA-3′ |
| K | 17 | 5′-NH2-C6-AAA AAA AAA AAA ATA ATC TAA |
| | | TTC TGG TCG CGG-3′ |
| K' | 18 | 5′-NH2-C6-AAA AAA AAA ACC GCG ACC AGA |
| | | ATT AGA TTA-3′ |
| L | 19 | 5′-NH2-C6-AAA AAA AAA AAA AGT GAT TAA |
| | | GTC TGC TTC GGC-3′ |
| L' | 20 | 5′-NH2-C6-AAA AAA AAA AGC CGA AGC AGA |
| | | CTT AAT CAC-3′ |
| M | 21 | 5′-NH2-C6-AAA AAA AAA AAA AGT CGA GGA |
| | | TTC GTA ACC TGT-3′ |
| M' | 22 | 5′-NH2-C6-AAA AAA AAA AAC AGG TTC AGA |
| | | ATC CTC GAC-3′ |
| N | 23 | 5′-NH2-C6-AAA AAA AAA AAA AGT CCT CGC |
| | | TTC GTC TAT GAG-3′ |
| N' | 24 | 5′-NH2-C6-AAA AAA AAA ACT CAT AGA CGA |
| | | AGC GAG GAC-3′ |
| O | 25 | 5′-NH2-C6-AAA AAA AAA AAA ACT TCG TGG |
| | | CTA GTC TGT GAC-3′ |
| O' | 26 | 5′-NH2-C6-AAA AAA AAA AGT CAC AGA CTA |
| | | GCC ACG AAG-3′ |
| P | 27 | 5′-NH2-C6-AAA AAA AAA AAA ATC GCC GTT |
| | | GGT CTG TAT GCA-3′ |
| P' | 28 | 5′-NH2-C6-AAA AAA AAA ATG CAT ACA GAC |
| | | CAA CGG CGA-3′ |
| Q | 29 | 5′-NH2-C6-AAA AAA AAA AAA ATA AGC CAG |
| | | TGT GTC GTG TCT-3′ |
| Q' | 30 | 5′-NH2-C6-AAA AAA AAA AGA CAC GAC ACA |
| | | CTG GCT TAT-3′ |

The second component of the B-RAP technology, which is also independent of the specific identities of any PCC candidates to be tested, is the library of DNA-bound SAC (SAC-DNA) conjugates used to assemble individual biotinylated PCC candidates onto specific barcode lanes. The SAC protein was conjugated with ssDNA strands complementary to the barcode DNA oligomers. This was done with N-succinimidly-4-formylbenzaldehyde (S-4FB) and maleimide 6-hydrazino-nicotinamide (MHPH), followed by fast protein liquid chromatography (FPLC) purification. An example FPLC trace for B′ SAC-DNA showed that the SAC-DNA elutes first followed by the excess unconjugated B′ ssDNA.

The performance of the library of fifteen SAC-DNAs (Shin et al., ChemPhysChem 2010, 11 (14), 3063-3069; Xue et al., J. Am. Chem. Soc. 2015, 137 (12), 4066-4069) was evaluated by hybridizing library elements onto the flow patterned ssDNA barcodes. The barcodes were then incubated with varying amounts of the fluorophore probe biotin-A₂₀-Cy3 (Biotin*, 50-400 nM). The resulting surface fluorescence was measured and compared to the fluorescence signal from the bottom edge barcode validation region. The fluorescent output with 532 nm excitation (F₅₃₂) of the captured biotinylated probe was lower than that of the validation region (45 to 65 k fluorescence units (f.u.)), likely reflecting the size of the SAC protein relative to the Cy3 fluorophore.

### KRas Protein Expression and Purification

The KRas protein isoform 4B was expressed from transformed BL21(D3) E. coli cells as a His₆-tagged protein (Boriack-Sjodin et al., Nature 1998, 394 (6691), 337-343) and purified by FPLC using a Ni-NTA resin. The fractions with pure KRas protein were dialyzed into tris-buffered saline (TBS, pH=7.4), aliquotted, and stored at −80.0° C. until needed.

### Preparation of Switch I and Switch II SynEps and Scrambled SynEps

The synthetic epitopes (SynEp1 and SynEp2) were 11-12 amino acid polypeptides with sequences extracted from the allosteric switch regions of KRas (FIG. 2 and Table 5). The SynEp1 differs from the wild-type sequence as it is missing a valine residue. An azido click handle was added by substituting residue-similar azido-amino acids, as shown in FIG. 2A. Rearranged version of the SynEps were also prepared and used in a pre-screen step to remove promiscuous binders. All epitopes were synthesized on biotin Novatag resin and purified using semi-preparative high-performance liquid chromatography (semi-prep HPLC). The appropriate fractions were identified using matrix-assisted laser desorption ionization time-of-flight mass spectrometry (MALDI-TOF MS).

The MALDI-TOF spectra for SynEp1 (FDEYD[P→^{4-N3}P]TIEDSY-PEG₄-Biotin: C₉₀H₁₂₈N₂₀O₃₃S (SEQ ID NO:31)) gave a peak corresponding to [M+Na], expected [M+Na]=2071.188 amu. The MADLI-TOF spectra for scrambled SynEp1 ([P→^{4-N3}P]EYDSIDDEFYT-PEG₄-Biotin: C₉₀H₁₂₈N₂₀O₃₃S (SEQ ID NO:32)) gave a peak corresponding to [M+2Pip-3 OH+H], expected [M+2Pip-3 OH+H]=2167.030 amu, which results from multiple piperidine and aspartamide adducts forming on the oligopeptide. The MALDI-TOF spectra for SynEp2 ([I→^{4-N3}K]LDTAGQEEYS-PEG₅-Biotin: C₇₇H₁₂₄N₂₀O₂₉S (SEQ ID NO:33)) gave peaks corresponding to [M+H], expected [M+H]=1825.864 amu, and to [M+Na], expected [M+Na]=1847.846 amu. The MALDI-TOF spectra for scrambled SynEp2 ([I→^{4-N3}K]LSTGEYDAQE-PEG₅-Biotin: C₇₇H₁₂₄N₂₀O₂₉S (SEQ ID NO:34)) gave a peak corresponding to the epitope with a single aspartamide formation [M-OH+H], expected [M-OH+H]=1809.99 amu. Each SynEp was dissolved in dimethyl sulfoxide (DMSO), quantified using a Nanodrop 2000 spectrophotometer, and stored at 4° C. until use.

TABLE 5
Synthetic epitopes and PCC ligands characterization table.

| | | HPLC | | | |
|---|---|---|---|---|---|
| | | Retention | Expected | Observed | |
| | Chemical | Time | mass | mass | SEQ ID |
| Amino Acid Sequence | Formula | (min) | (amu) | (amu) | NO |
| FDEYD[P→^{4-N3}P]TIEDSY- | C₉₀H₁₂₈N₂₀O₃₃S | 32:30- | [M + H] = | 2074.32 | 31 |
| PEG₄-Biotin | | 33:30 | 2049.88 | | |
| | | | [M + Na] = | | |
| | | | 2071.188 | | |

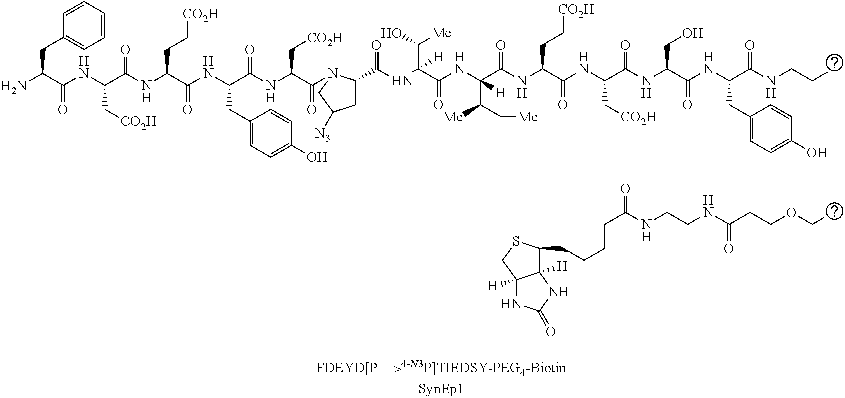 [SMILES: CCCNC(=O)[C@H](Cc1ccc(O)cc1)NC(=O)[C@H](CO)NC(=O)[C@H](CC(=O)O)NC(=O)[C@H](CCC(=O)O)NC(=O)[C@@H](NC(=O)[C@@H](NC(=O)[C@@H]1CC(N=[N+]=[N-])CN1C(=O)[C@H](CC(=O)O)NC(=O)[C@H](Cc1ccc(O)cc1)NC(=O)[C@H](CCC(=O)O)NC(=O)[C@H](CC(=O)O)NC(=O)[C@@H](N)Cc1ccccc1)[C@@H](C)O)[C@H](C)CC ; CCOCCC(=O)NCCNC(=O)CCCC[C@@H]1SC[C@@H]2NC(=O)N[C@@H]21]

Structure

| | | | | | |
|---|---|---|---|---|---|
| [I→^{4-N3}K]LDTAGQEEYS- | C₇₇H₁₂₄N₂₀O₂₉S | 28:30- | [M + H] = | 1826.222 | 33 |
| PEG₅-Biotin | | 29:30 | 1825.864 | | |

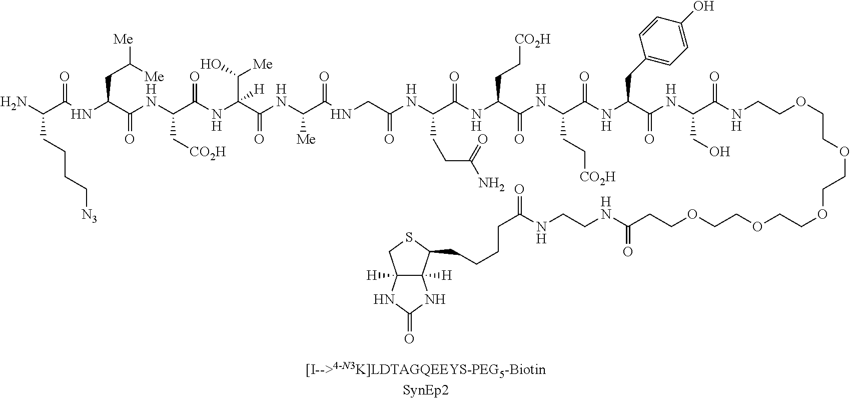 [SMILES: CC(C)C[C@H](NC(=O)[C@@H](N)CCCCN=[N+]=[N-])C(=O)N[C@@H](CC(=O)O)C(=O)N[C@H](C(=O)N[C@@H](C)C(=O)NCC(=O)N[C@@H](CCC(N)=O)C(=O)N[C@@H](CCC(=O)O)C(=O)N[C@@H](CCC(=O)O)C(=O)N[C@@H](Cc1ccc(O)cc1)C(=O)N[C@@H](CO)C(=O)NCCOCCOCCOCCOCCOCCC(=O)NCCNC(=O)CCCC[C@@H]1SC[C@@H]2NC(=O)N[C@@H]21)[C@@H](C)O]

Structure

| | | | | | |
|---|---|---|---|---|---|
| [P→^{4-N3}P]EYDSIDDEFYT- | C₉₀H₁₂₈N₂₀O₃₃S | 32:00- | [M + H] = | 2166.39 | 32 |
| PEG₄-Biotin | | 33:00 | 2049.875 | | |
| | | | [M + 2 Pip- | | |
| | | | 3 OH + H] = | | |
| | | | 2167.030 | | |

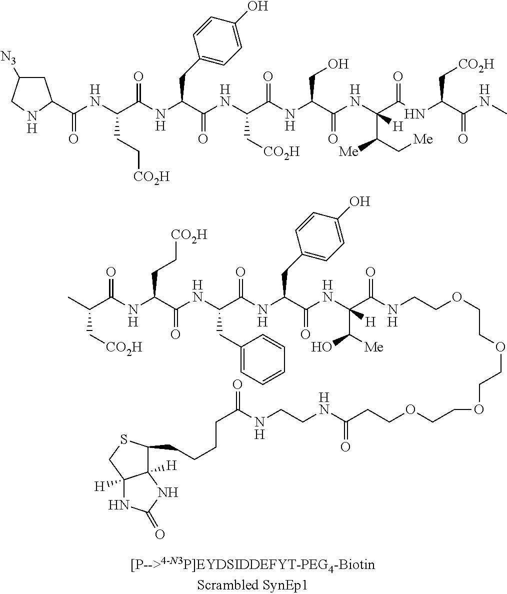

Structure

| | | | | | |
|---|---|---|---|---|---|
| [I→^{4-N3}K]LSTGEYDAQE- | C₇₇H₁₂₄N₂₀O₂₉S | 25:00- | [M + H] = | 1810.51 | 34 |
| PEG₅-Biotin | | 26:00 | 1825.649, | | |
| | | | [M − OH + H] = | | |
| | | | 1809.991 | | |

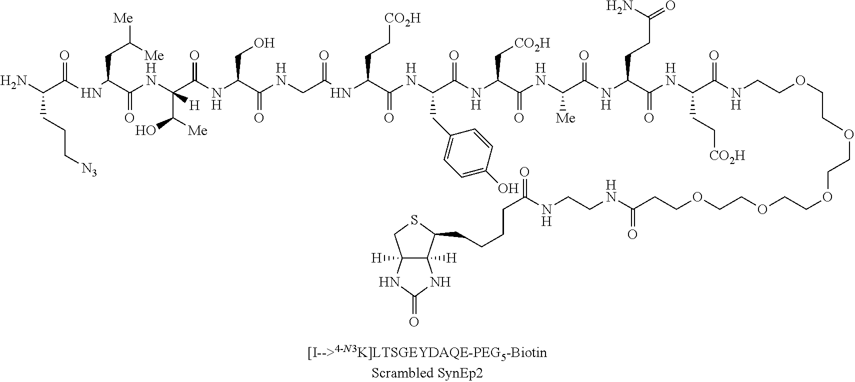 [SMILES: CC(C)C[C@H](NC(=O)[C@@H](N)CCCN=[N+]=[N-])C(=O)N[C@H](C(=O)N[C@@H](CO)C(=O)NCC(=O)N[C@@H](CCC(=O)O)C(=O)N[C@@H](Cc1ccc(O)cc1)C(=O)N[C@@H](CC(=O)O)C(=O)N[C@@H](C)C(=O)N[C@@H](CCC(N)=O)C(=O)N[C@@H](CCC(=O)O)C(=O)NCCOCCOCCOCCOCCOCCC(=O)NCCNC(=O)CCCC[C@@H]1SC[C@@H]2NC(=O)N[C@@H]21)[C@@H](C)O]

Structure

| | | | | | |
|---|---|---|---|---|---|
| HGIVG | C₅₇H₉₅N₁₇O₁₅S | 3:30-4:30 | [M + H] = | 1310.81 | 35 |
| | | (f1), | 1290.699 | (f1), | |
| | | 36:00- | [M + Na] = | 1289.31 | |
| | | 38:00 (f2) | 1312.681 | (f2) | |

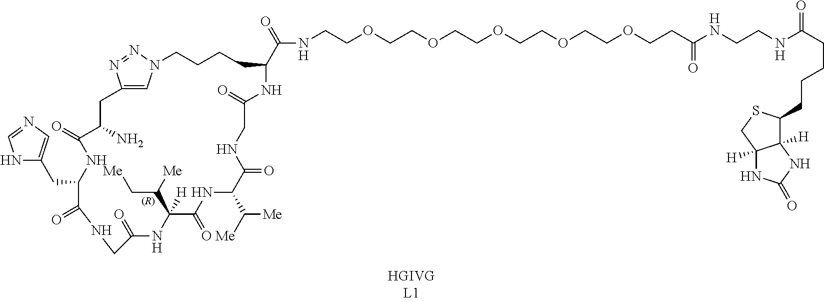

Structure

| | | | | | |
|---|---|---|---|---|---|
| EGVPV | C₅₈H₉₇N₁₅O₁₇S | 2:30-5:00 | [M + H] = | 1329.9 | 36 |
| | | | 1306.699 | | |
| | | | [M + Na] = | | |
| | | | 1330.682 | | |

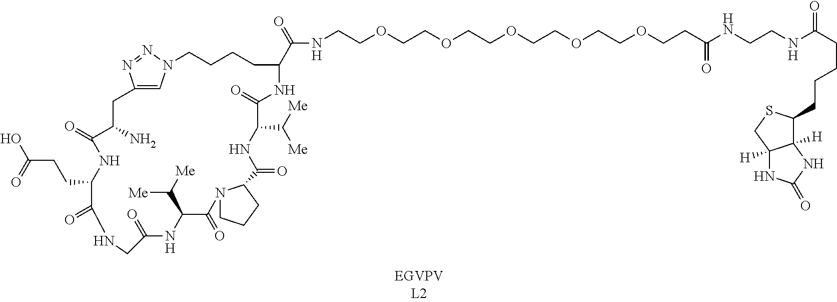 [SMILES: CC(C)[C@@H]1NC(=O)[C@@H]2CCCN2C(=O)[C@H](C(C)C)NC(=O)CNC(=O)[C@H](CCC(=O)O)NC(=O)[C@@H](N)Cc2cn(nn2)CCCCC(C(=O)NCCOCCOCCOCCOCCOCCC(=O)NCCNC(=O)CCCC[C@@H]2SC[C@@H]3NC(=O)N[C@@H]32)NC1=O]

Structure

| | | | | | |
|---|---|---|---|---|---|
| GEVVP | C₅₈H₉₇N₁₅O₁₇S | 2:30-5:00 | [M + H] = | 1330.16 | 37 |
| | | (f1), | 1306.699 | (f1), | |
| | | 41:30- | [M + Na] = | 1312.34 | |
| | | 43:00 (f2) | 1330.681 | (f2) | |

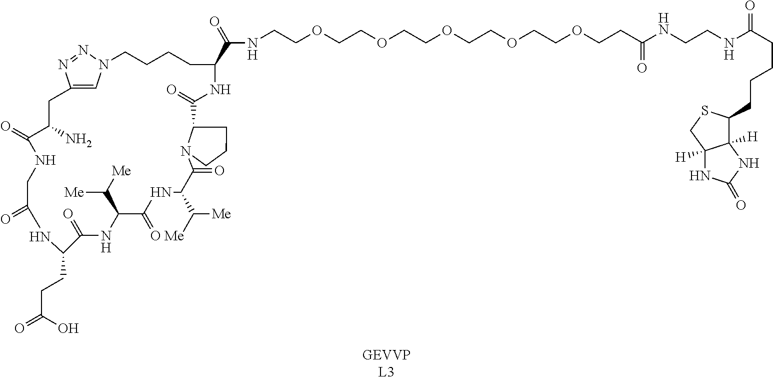 [SMILES: CC(C)[C@@H]1NC(=O)[C@H](CCC(=O)O)NC(=O)CNC(=O)[C@@H](N)Cc2cn(nn2)CCCC[C@@H](C(=O)NCCOCCOCCOCCOCCOCCC(=O)NCCNC(=O)CCCC[C@@H]2SC[C@@H]3NC(=O)N[C@@H]32)NC(=O)[C@@H]2CCCN2C(=O)[C@H](C(C)C)NC1=O]

Structure

| | | | | | |
|---|---|---|---|---|---|
| VEVPY | C₆₅H₁₀₃N₁₅O₁₈S | 36:30- | [M + H] = | 1414.38 | 38 |
| | | 39:30 (f1), | 1414.740 | (f1), | |
| | | 40:00- | [M + Na] = | 1435.89 | |
| | | 41:00 (f2) | 1436.722 | (f2) | |

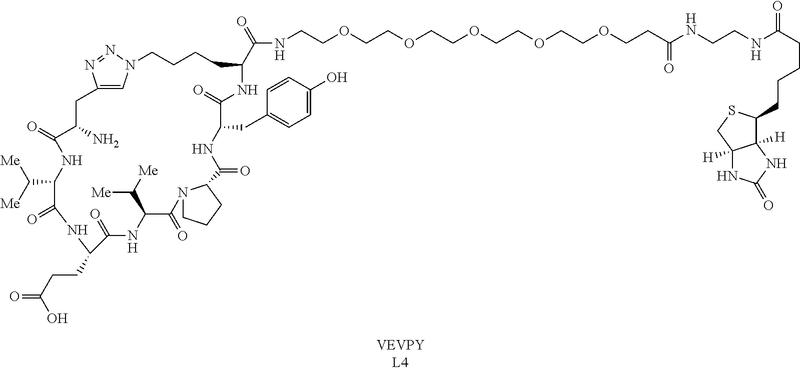 [SMILES: CC(C)[C@@H]1NC(=O)[C@@H](N)Cc2cn(nn2)CCCC[C@@H](C(=O)NCCOCCOCCOCCOCCOCCC(=O)NCCNC(=O)CCCC[C@@H]2SC[C@@H]3NC(=O)N[C@@H]32)NC(=O)[C@H](Cc2ccc(O)cc2)NC(=O)[C@@H]2CCCN2C(=O)[C@H](C(C)C)NC(=O)[C@H](CCC(=O)O)NC1=O]

Structure

| | | | | | |
|---|---|---|---|---|---|
| VEVYP | C₆₅H₁₀₃N₁₅O₁₈S | 37:00- | [M + H] = | 1413.47 | 39 |
| | | 39:00 (f1), | 1414.740 | (f1), | |
| | | 39:00- | [M + Na] = | 1435.88 | |
| | | 40:00 (f2) | 1436.721 | (f2) | |

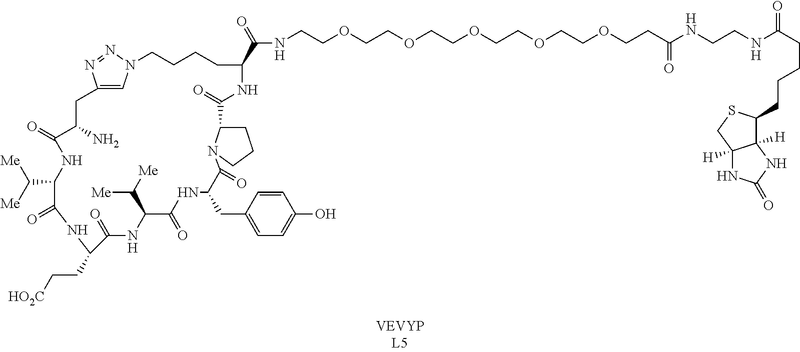 [SMILES: CC(C)[C@@H]1NC(=O)[C@H](CCC(=O)O)NC(=O)[C@H](C(C)C)NC(=O)[C@@H](N)Cc2cn(nn2)CCCC[C@@H](C(=O)NCCOCCOCCOCCOCCOCCC(=O)NCCNC(=O)CCCC[C@@H]2SC[C@@H]3NC(=O)N[C@@H]32)NC(=O)[C@@H]2CCCN2C(=O)[C@H](Cc2ccc(O)cc2)NC1=O]

Structure

| | | | | | |
|---|---|---|---|---|---|
| VTVPY | C₆₄H₁₀₃N₁₅O₁₇S | 37:30- | [M + H] = | 1387.36 | 40 |
| | | 39:00 | 1386.746 | | |
| | | | [M + Na] = | | |
| | | | 1408.727 | | |

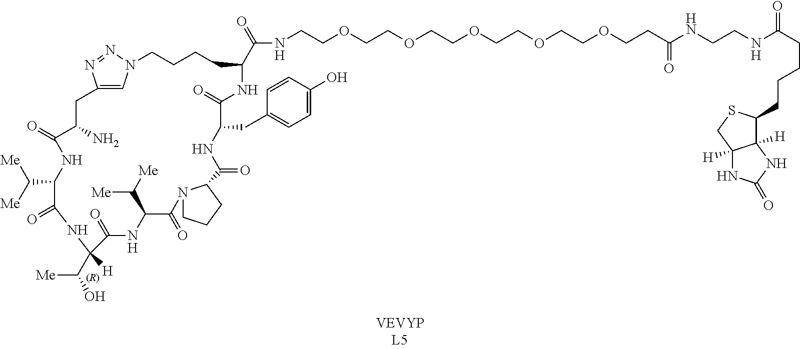

Structure

| | | | | | |
|---|---|---|---|---|---|
| VQVPY | C₆₅H₁₀₄N₁₆O₁₇S | 36:00- | [M + H] = | 1412.29 | 41 |
| | | 38:30 (f1), | 1413.722 | (f1), | |
| | | 39:00- | [M + Na] = | 1434.80 | |
| | | 41:30 (f2) | 1435.738 | (f2) | |

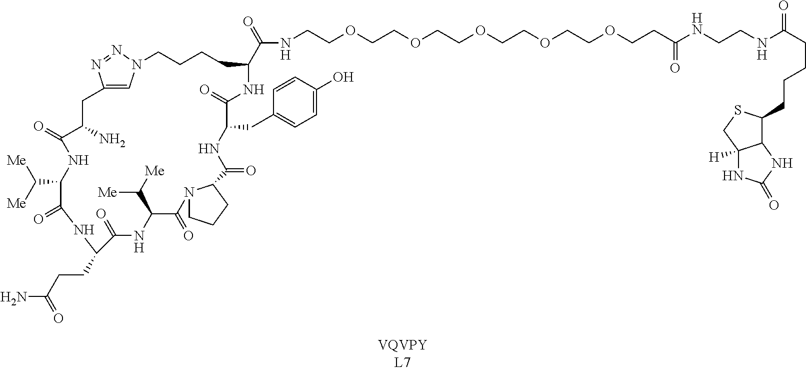 [SMILES: CC(C)[C@@H]1NC(=O)[C@@H](N)Cc2cn(nn2)CCCC[C@@H](C(=O)NCCOCCOCCOCCOCCOCCC(=O)NCCNC(=O)CCCC[C@@H]2SC[C@@H]3NC(=O)NC32)NC(=O)[C@H](Cc2ccc(O)cc2)NC(=O)[C@@H]2CCCN2C(=O)[C@H](C(C)C)NC(=O)[C@H](CCC(N)=O)NC1=O]

Structure

| | | | | | |
|---|---|---|---|---|---|
| TDNFY | C₆₆H₉₈N₁₆O₂₀S | 36:30- | [M + H] = | 1488.37 | 42 |
| | | 38:00 | 1467.694 | | |
| | | | [M + Na] = | | |
| | | | 1489.676 | | |

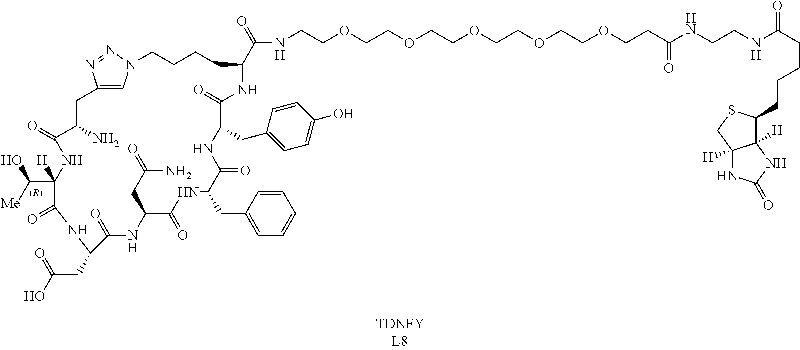

Structure

| | | | | | |
|---|---|---|---|---|---|
| QDNFY | C₆₇H₉₉N₁₇O₂₀S | 4:30-5:30 | [M + H] = | 1514.14 | 43 |
| | | (f1), | 1494.705 | (f1), | |
| | | 37:00- | [M + Na] = | 1494.04 | |
| | | 38:30 (f2) | 1516.687 | (f2) | |

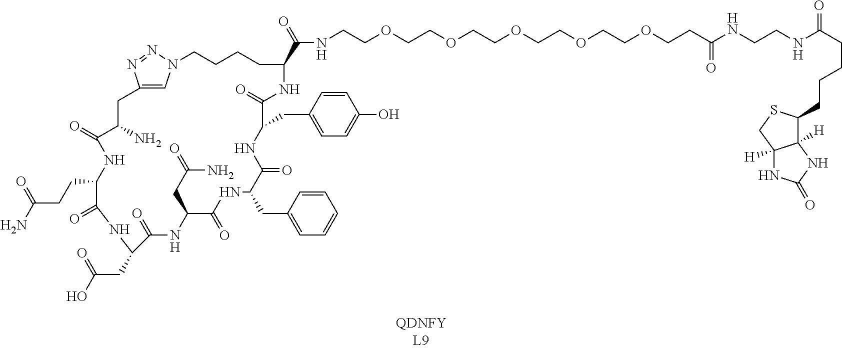 [SMILES: NC(=O)CC[C@@H]1NC(=O)[C@@H](N)Cc2cn(nn2)CCCC[C@@H](C(=O)NCCOCCOCCOCCOCCOCCC(=O)NCCNC(=O)CCCC[C@@H]2SC[C@@H]3NC(=O)N[C@@H]32)NC(=O)[C@H](Cc2ccc(O)cc2)NC(=O)[C@H](Cc2ccccc2)NC(=O)[C@H](CC(N)=O)NC(=O)[C@H](CC(=O)O)NC1=O]

Structure

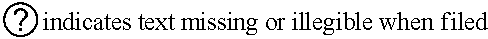

Expected masses were calculated using the mass calculator at web site lfd.uci.edu/˜gohlke/molmass/?q=C152H224N32O38S2Na.

### Library Preparation and In-Situ Library Click Screen

A comprehensive OBOC library of 5-mer variable peptide macrocycles, using an 18 amino acid basis set, was prepared as previously reported (Das et al., Angew. Chemie Int. Ed. 2015, 54 (45), 13219-13224). The macrocyclic peptides were closed with a 1,4 triazole using Cu(I)-catalyzed click chemistry. These macrocycles were designed to present a propargylglycine click handle. The in situ library click screen was a dual SynEp version of a previously reported protocol (Das et al., Angew. Chemie Int. Ed. 2015, 54 (45), 13219-13224). After removing the beads that bound to the scrambled SynEps during a pre-clear screen the remaining library was incubated with both SynEp1 and SynEp2 (Supplementary Experimental Methods). After incubating with an anti-biotin capture antibody and an alkaline-phosphatase conjugated secondary antibody, the hit beads were identified by their deep purple color. The isolated hit beads were stored at RT in 0.1 M hydrochloric acid. Just prior to sequencing by Edman degradation, the beads were decolorized in N-methyl 2-pyrrolidone (NMP) (Table 5). The hit compounds were then scaled up on biotin Novatag resin following previously established protocols (Das et al., Angew. Chemie Int. Ed. 2015, 54 (45), 13219-13224), purified, lyophilized, reconstituted in DMSO, quantified, and then stored at 4° C. until ready for use.

### Surface Immunofluorescent Assays on the Barcoded Rapid Assay Platform

The barcode patterned microchip surface was partitioned into 16 individual microwells using a pre-fabricated PDMS slab. Individual biotinylated PCC candidates were complexed to specific SAC-DNA conjugates, combined into a cocktail, and then self-assembled, via DNA hybridization, onto designated barcode stripes (FIGS. 2-4). Incubation with a specific concentration of the target protein preceded incubation with a primary capture antibody and then a fluorophore-conjugated secondary detection antibody. During assay execution, each well represents a different target concentration or assay condition. Once developed, the fluorescence of the barcodes is digitized using a GenePix 4400A array scanner, with an excitation laser power optimized to a power level of 40% (60 W), which maximizes detection sensitivity while also minimizing signal saturation. Data extraction occurs using 10 μm radius circles, taken along the length of a barcode stripe. A fluorescence signal representing the average of all the pixels within a given circle is collected. A total of ten circles (data-blocks) are measured along a 180 μm span of the middle portion for each individual barcode lane in a given well. 10 μM KRas protein gave an average F₆₃₅ of 47,868.7, with a standard deviation of 4154.53. 1 μM KRas protein gave an average F₆₃₅ of 11437.6, with a standard deviation of 1602.202. 100 nM KRas protein gave an average F₆₃₅ of 1780.6, with a standard deviation of 236.38. This illustrates the intensity across a stripe compared to the intensity from the data-block extraction. After extraction the data is background corrected. The background signal arises from (a) non-specific binding of the primary and secondary antibodies (independent of [KRas]), but can vary across different barcode stripes), and (b) non-specific binding of KRas protein ([KRas] dependent). Background (a) was assessed by measuring the average signal in the null protein well for each stripe. Background (b) was assessed by measuring the average fluorescence for the dummy ligand (Biotin* probe) that was in each well. The background subtracted data was then graphed in Graphpad Prism 7 and fitted to a sigmoidal curve (Hill coefficient=1).

### Measuring the Effect of the Allosteric Ligands on the Intrinsic KRas GTPase Activity

KRas inhibition assays were carried out using a GTPase Glo Assay kit from Promega. Each candidate inhibitor PCC was initially tested by combining a concentration series of the ligand with 10 μM KRas protein in an opaque white 96-well plate and incubated with 5 μM 5′-guanosine triphosphate (GTP) for two hours. The remaining GTP was minute incubation with the detection reagent. Chemiluminescence was measured using a Flexstation 3 plate reader (All wavelengths mode, 500 ms integration), and plotted using Graphpad Prism 7. A full inhibition curve of the most potent inhibitor was then generated using a four-hour incubation with GTP and a 2.5 μM to 100 μM concentration range. All measurements were done in triplicate.

### DNA Barcode Chip Patterning and Validation

The DNA barcode chips were prepared by micro channel-guided flow patterning as described in Yu et al., Annu. Rev. Anal. Chem. 2014, 7 (1), 275-295 and Wei et al., Cancer Cell 2016, 29 (4), 563-573. A PDMS slab having the micro-channels was made by soft lithography on a silicon wafer. Its mold was designed as FIG. 7 and prepared with SU8 2025 negative photoresist. The fabricated mold contained microfluidic circuits of 20 parallel channels with 50 μm width and ˜40 μm height. Sylgard® 184 PDMS pre-polymer and curing agents were mixed in a 10:1 ratio, degassed, ˜60 g of the mixture poured onto the mold, and baked for two hours at 80° C. for curation. The cured PDMS slab was peeled off from the mold, cut into individual microfluidic molds, and the inlet and outlet holes of the microfluidic circuits were punched with the sizes of two mm and 0.5 mm respectively. The number of the inlets and outlets punched out were determined by the number of single stranded DNAs (ssDNAs) used in the assay, and fifteen orthogonal ssDNAs (B-Q, Table 4) were used in this study. The slab was then aligned with a PLL glass slide, and bonding occurred with baking at 80° C. for two hours. After cooling briefly, the inlet wells were loaded with 3 μL of a PLL solution (0.1% (^{m}/ₘ) in H₂O), and the PLL solution was flowed and dried by 13.8 kPa nitrogen gas blowing through the solution-loading device overnight. The next day, C6 amine-modified DNA solutions (300 μM in (3:2 (^{v}/ᵥ)) PBS/DMSO) were individually mixed (1:1) with a 2 mM BS3 cross-linker solution in PBS. Each freshly prepared mixture was flown through a channel under 13.8-20.6 kPa of nitrogen gas using the solution-loading device for 1 hour, and then only the assembled PDMS slab and the bonded PLL slide was incubated at room temperature for 2 hours in a humidified chamber. After incubation, the PDMS slab was removed, and the DNA patterned PLL slides were washed with a 0.02% aqueous SDS solution, doubly distilled water (MQ H₂O) (3×), and spun dry.

To validate the DNA barcode chips, a 5′-modified Cy3-labelled complementary ssDNA cocktail was prepared in 1% BSA in PBS (50 nM each ssDNA). The validation occurred over two rounds (B, D, F, H, K, N, P, M then C, E, G, I, L, O, Q) in order to check for channel leaks and crossover. A 120 μL aliquot of the validation solution was applied to a small region at the bottom edge of the DNA barcode before incubating at 37° C. for one hour. After incubation, this region was washed with 1% BSA in PBS followed by PBS (2×), and the slide was spun dry before being scanned by Axon GenePix 4400A (532 nm, PMT 450, Power 15% (23 W)) (FIGS. 5 and 6A).

### Expression of Cysteine-Modified Streptavidin (SAC) Protein

The SAC protein was expressed using a modification of the procedure reported by Sano and Cantor (Proc. Natl. Acad. Sci. 1990, 87 (1), 142-146). A 100 mL starter culture of autoclaved LB media (10.0 g Tryptone, 5.00 g yeast, 10.0 g NaCl per L H₂O) was prepared by inoculating with 50 μL of 100 ^{mg}/_{mL} of ampicillin (final concentration 50 ^{μg}/_{mL}) and 100 μL of 34 ^{mg}/_{mL} chloramphenicol (final concentration 34 ^{μg}/_{mL}) followed by a sterile pipet scraping of a 50% (^{v}/ᵥ) glycerol stock containing transformed E. coli BL21(D3) cells. The starter culture incubated overnight at 37° C. and 250 RPM before adding 10.0 mL of starter culture aliquots to six 2800 mL Fernbach-Style Culture Flasks containing 1.00 L autoclaved LB media with 500 μL of 100 ^{mg}/_{mL} of ampicillin (final concentration 50 ^{μg}/_{mL}), 1000 μL of 34 ^{mg}/_{mL} chloramphenicol (final concentration 34 ^{μg}/_{mL}), and 1000 μL of 40% (^{w}/_{w}) autoclaved glucose (final concentration 0.4% (^{w}/_{w}). The flasks were left to culture at 37.0° C., 250 RPM until A₆₈₀=0.500, and induction was triggered with 1000 μL of a 400 mM 1,6-IPTG solution (final concentration 400 μM). Expression continued at 37.0° C., 250 RPM for four hours before spinning down the cells at 6000 RPM, 5 minutes at 4° C. The cells were resuspended in 50 mL of a 10 mM Tris, 1 mM EDTA, 130 mM NaCl buffer at pH=8.0 and spun down (2×). The cells were then flash frozen in N₂₀) and stored at −80.0° C. until needed.

Isolation of SAC Inclusion Bodies from E. coli Cells

The cell pellet was thawed in ice before resuspending in two 50-mL falcon tubes with 40 mL of TEX buffer (30 mM Tris, 2 mM EDTA, 0.1% TritonX). Each tube was charged with 40 mg fresh lysozyme powder (Final concentration 1.0 ^{mg}/_{mL}), vortexed until mixed, and allowed to lyse for 30 min while tumbling at RT. The solution was very viscous after lysis. The DNA and RNA were degraded by adding 400 μL of 10 ^{mg}/_{mL} DNAse and 10 ^{mg}/_{mL} RNAse in TE Buffer (10 mM Tris, 130 mM NaCl, 1 mM EDTA) (final concentration 10 ^{μg}/_{mL}), 960 μL of 500 mM MgCl₂ (final concentration 12 mM), and 40 μL of 1 M MnCl₂ (final concentration 1 mM) to each tube of cell lysate, and the solution was allowed to digest for 30 minutes while tumbling at RT. After digestion, the solution was spun down at 7800 RPM, RT for 10 minutes. The resulting inclusion body (IB) pellets were both washed in 40 mL TEX buffer and spun down at 7800 RPM, 5 minutes at RT. Pellets were washed with 40 mL buffer minus Triton X again before spinning down at 7800 RPM, 5 minutes at RT once more. Each pellet was taken up in 10 mM Tris and spun down at 7800 RPM, 10 minutes at RT, aliquotted, and stored at −80.0° C. until needed. If the final pellet is light brown then some DNA is still present. This will be removed at the beginning of the refolding procedure.

### Refolding and Purification of SAC Protein

The procedure described here is a modification of the procedure developed by Sano and Cantor (Proc. Natl. Acad. Sci. 1990, 87 (1), 142-146). After the initial denaturing keep all solutions at 4° C.

An IB aliquot was dissolved in 1000 μL denaturing buffer (6 M Guanidine.HCl at pH=1.5 with 10 mM βME), vortexed, spun down at 13,000 RPM, 2 min at RT, and filtered using a 0.45 μM low-protein binding filter. The resulting solution should be clear and nearly colorless. The A₂₈₀ was measured on a Nanodrop2000 spectrophotometer, and the concentration of denatured SAC monomer was calculated (internet site biotools.nubic.northwestern.edu/proteincalc.html). The denatured SAC solution was diluted to 1000 μL in denaturing buffer and added dropwise to a rapidly stirring solution of refolding buffer (50 mM NH₄OAc, 150 mM NaCl, and 10 mM (ME at pH=6.0) (Final [denatured SAC] ˜4 μM). The stir rate was then decreased to about half of its original value, and the solution was covered by aluminum foil to refold overnight. After sterile filtration with a 0.45 μm low-protein binding filter the resulting solution was concentrated to 10-15 mL using Amicon Millipore filters (10,000-30,000 MWCO) before dialyzing the refolded SAC protein in buffer A (50 mM NaHCO₃, 500 mM NaCl, 10 mM βME at pH=11.0) until the solution had a pH of ˜11 (about 2 hours). The crude protein was then diluted (1:1) with buffer A, mixed with 2 mL of 2-iminobiotin agarose resin, and allowed to incubate with tumbling in the cold room for one hour. After incubation the supernatant was eluted (3×) before eluting with buffer A until the A₂₈₀ went to baseline. Pure SAC was eluted with buffer B (50 mM NaOAc, 50 mM NaCl at pH=4.0) until the A₂₈₀ went to baseline again. Fractions with pure SAC were pooled and dialyzed against a PBS solution (PBS, 10 mM βME, pH=7.5) overnight. The SAC was concentrated to ˜1 ^{mg}/_{mL} final concentration, divided into 100 μL aliquots, and stored at −80° C.

### Preparation of the DESL SAC-DNA Conjugates

For each planned SAC-DNA conjugation, two Zeba columns were prepared (3×300 μL of 5 mM TCEP in PBS, 3.9 k RPM, 1 min at RT). Each 100 μL aliquot of SAC was desalted in two separate Zeba columns to remove the βME (3.9 k RPM, 2 min at RT). After transferring to eppendorf tubes, 6 μL anhydrous DMF was added followed by 6 μL MHPH (100 mM in anhydrous DMF). Separate eppendorf tubes were charged with 80 μL of 500 μM of conjugation ssDNA in PBS followed by 15 μL anhydrous DMF and 20 μL S-4FB (100 mM in anhydrous DMF). The SAC and DNA solutions were vortexed gently, briefly spun down, and left to react at RT in the dark for four hours. For each conjugation in progress, four Zeba columns were buffered exchanged with citrate buffer (150 mM NaCl, 50 mM sodium citrate, pH=6.0) (3×300 μL citrate buffer, 3.9 k RPM, 1 min at RT). The SAC and DNA solutions were desalted separately (2×3.9 k RPM, 2 min, at RT) before combining each SAC protein aliquot with a unique ssDNA solution. The solutions were vortexed gently, briefly spun down, and left to react in the dark at RT overnight. The reactions were quenched by placing at 4° C. Each SAC-DNA conjugate was purified by FPLC using a Superdex75 Increase column (isocratic in PBS, 0.5 ^{mL}/ₘᵢₙ, 0.5 mL fractions, 75 minutes). Fractions containing pure SAC-DNA were pooled and concentrated using Amicon Ultra-4 Centrifugal filters (30 k MWCO): 3900 RPM, 30 minutes at 4° C. The concentrated SAC-DNA proteins were quantified (web site idtdna.com/calc/analyzer) using a Nanodrop2000 spectrophotometer in the ssDNA nucleic acid mode (using two for the average number of ssDNA strands conjugated as previously established) and stored at 4° C. until needed.

### DESL Set Biotin Binding Capacity Validation Protocol

### Buffers Used:

Wash buffer: PBS+0.05% Tween20 (PBST)

Blocking Buffer: PBS+1% BSA

Wash steps used 50 ^{μL}/_{well}

Incubation steps used 30 ^{μL}/_{well}

After loading the Biotin* probe change pipette tips every time that you aspirate or add solution to a well to prevent cross contamination.

A prefabricated PDMS template was aligned onto the DNA barcode and the microchip slide was taped into a 10 cm petri dish. The wells were washed with PBST before loading blocking buffer and placing the platform into a 37° C. incubator for one hour. A cocktail containing 50 nM of each SAC-DNA in PBS was prepared and added to the pre-blocked wells. The SAC-DNA conjugates were allowed to hybridize to the DNA barcode at 37° C. for one hour before washing the wells with PBST (3×). Each well was loaded with 50 nM, 100 nM, 150 nM, 200 nM, 300 nM, or 400 nM Biotin* in PBS, and the platform was left to shake covered at RT for one hour. The wells were washed with PBST (3×) before peeling off the PDMS slab and dipping the barcode into PBS, (1:1) PBS/MQ H₂O, MQ H₂O (2×). The barcode was then spun dry and read on the Genepix (532 nm, PMT 450, Power 15% (23 W)).

### Expressing and Purifying WT KRas Protein

The KRas protein was expressed and purified using a modification of the procedure reported by Kuriyan (Boriack-Sjodin et al., Nature 1998, 394 (6691), 337-343). A starter culture of 100 mL of autoclaved LB media was inoculated with 100 μL of 100 ^{mg}/_{mL} of ampicillin (final concentration 100 ^{μg}/_{mL}) followed by a scraping of a 25% (^{v}/ᵥ) glycerol stock containing transformed E. coli (BL21(DE3)) cells. The starter culture was left in an incubator at 37.0° C., 250 RPM overnight before adding 10.0 mL starter culture aliquots to six 2800 mL Fernbach-Style Culture Flasks containing 1.00 L autoclaved LB media with 1000 μL of 100 ^{mg}/_{mL} of ampicillin (final concentration 100 ^{μg}/_{mL}). The flasks were left to culture at 37.0° C., 250 RPM until A₆₈₀=0.500-0.600 and induction was triggered with 1000 μL of a 250 mM 1,6-IPTG solution (final concentration 250 μM). The cells were then left to express overnight at 18.0° C., 250 RPM before being spun down, resuspended in buffer A (20 mM Tris, 500 mM NaCl, 20 mM imidazole, 5 mM MgCl₂, pH=8.0), flash frozen in N₂₍ₗ₎), and stored at −80.0° C. until needed. After thawing and douncing, the cells were lysed using a cell disruptor, the cell wall lysate spun down at 8000 RPM, 4° C. for 20 minutes, sterile filtered with a 0.45 μm low-protein binding filter, and purified using FPLC with a Ni-NTA superflow cartridge and a gradient of buffer A to buffer B (20 mM Tris, 300 mM NaCl, 250 mM imidazole, 5 mM MgCl₂, pH=8.0). Fractions containing pure KRas were pooled and dialyzed against Tris buffered saline (TBS) (25 mM Tris, 150 mM NaCl, 10 mM MgCl₂, pH=7.5) overnight. The resulting solution was concentrated using Amicon Ultra-15 centrifugal filters (10 k MWCO), quantified (internet site biotools.nubic.northwestern.edu/proteincalc.html), separated into aliquots, flash frozen in N₂₍ₗ₎, and stored at −80.0° C. until needed.

### In Situ Library Click Screen Combined Preclear/Anti-Screen

The in-situ click dual SynEp library screen followed a procedure similar to the one outlined in Das et al., Angew. Chemie Int. Ed. 2015, 54 (45), 13219-13224, using 450 mg of Pra-capped one-bead-one-compound (OBOC) library. Blocking was performed overnight at 4° C. with blocking buffer (1% BSA and 0.1% Tween20 in TBS). After washing with blocking buffer (3×3 minutes) incubation with 25 μM of each scrambled SynEp in binding buffer (0.1% BSA and 0.1% Tween20 in TBS) occurred overnight at 4° C. The library was washed with TBS (3×1 minute) then stripped with 7.5 M Gua.HCl (pH=2.0) at RT for one hour to remove any non-covalently bound scrambled SynEps. Ten rinses with TBS preceded another incubation with blocking buffer at RT for one hour. After five quick rinses of the library with blocking buffer the library was incubated with a cocktail of a (10,000:1) dilution of the mouse anti-biotin-alkaline phosphatase conjugated ab (#A6561), (1,000:1) dilution of the rabbit anti-Ras ab (CST #3965), and a (10,000:1) dilution of the goat anti-rabbit-alkaline phosphatase ab (#A8025) in binding buffer to perform the preclear and anti-screen in one assay. Washes (5×3 minutes) with a high salt buffer (25 mM Tris.HCl, 10 mM MgCl₂, 700 mM NaCl, pH=7.4), and a low salt buffer (5×3 minutes) (25 mM Tris.HCl, pH=7.4). The developing buffer was prepared with 66 μL of BCIP (50 ^{mg}/_{mL} in 70% DMF) in 10 mL of developing buffer (100 mM Tris-HCl, 150 mM NaCl, 1 mM MgCl₂) and incubated with the library beads in a 20 cm petri dish for ten minutes before adding 66 μL of NBT (50 ^{mg}/_{mL} in 70% DMF) and incubating for an additional fourteen minutes. The beads were then washed 5× with TBS, and stored in 0.1 M HCl_{(aq)} in a 20 cm petri dish. Any beads that turned purple during the combined preclear/anti-clear were promiscuous binders and consequently were picked out using a 10-μL micropipette and discarded. After removing all of the sticky beads the remaining beads were washed with 7.5 M Guan.HCl (pH=2.0) for 30 minutes, rinsed with MQ H₂O (10×), and incubated in NMP overnight to remove any trace purple coloring. Final rinses with MQ H₂O (3×), TBS (7×) preceded an overnight incubation at 4° C. with blocking buffer.

### Library Click Screen Product/Target Screens

The pre-blocked library was washed with blocking buffer (3×5 minutes) before loading 25 μM of each SynEp in binding buffer and incubating at RT overnight. After rinsing with TBS (3×) the library was incubated with 7.5 M Guan.HCl (pH=2.0) for one hour at RT before rinsing with TBS (10×). The library then underwent an additional one hour incubation with blocking buffer at RT before rinsing with blocking buffer (5×), and incubating with a (10,000:1) dilution of the mouse anti-biotin-alkaline phosphatase conjugated ab in binding buffer for one hour at RT. Development of the library followed the same procedure as the preclear/anti-screen, and the darkest beads were set aside for Edman degradation sequencing. The remaining ˜50 light purple beads from the product screen were prepped following the same procedure after the preclear/anti-screen and screened again, using appropriately scaled amounts of reagents, against 25 μM of the full-length KRas protein. After developing additional beads were picked for a total of seven dark beads from the product/target screens of which five beads yielded readable sequences by Edman degradation sequencing.

### Peptide Preparation and Purification

All cyclic peptides and epitopes were prepared following the procedures outlined in Das et al., Angew. Chemie Int. Ed. 2015, 54 (45), 13219-13224.

The peptides and epitopes were isolated using the following procedure. The resin was rinsed with DCM (5×) and dried under vacuum. A 20 mL scintillation vial was charged with a stir-bar, resin, and 3-5 mL cleavage solution (95% TFA, 2.5% TESH, 2.5% H₂O) and allowed to stir at room temperature for 2-2.5 hours. The solution was then filtered into 40 mL of cold diethyl ether, vortexed for 10 seconds, and stored at 4° C. overnight. The precipitated protein was centrifuged into a pellet at 4500 RPM for 10-15 minutes prior to decantation of the supernatant. The crude peptides were dissolved in either DMSO or (1:1) MeCN/H₂O w/0.1% TFA before HPLC purification, and lyophilization of desired fractions. The resulting lyophilized powder was dissolved in DMSO, quantified (internet site biotools.nubic.northwestern.edu/proteincalc.html), and stored at 4° C. when not in use.

Measurement of PCC Ligand KRas EC₅₀ with the Barcode Rapid Assay Platform

### Buffers Used:

Wash buffer: PBS+0.05% Tween20 (PBST)

Blocking Buffer: PBS+1% BSA

Protein Incubation Buffer: Tris-buffered saline (TBS)+0.05% Tween20 (TBST)

1° ab buffer: PBS+5% BSA

2° ab buffer: PBS+1% BSA

Wash steps used 50 ^{μL}/_{well}

Incubation steps used 30 ^{μL}/_{well}

The plate must be covered during incubation steps to protect the fluorescent blank

After loading the KRas protein change tips every time that solution is aspirated or added to a well to prevent cross-contamination

A pre-fabricated PDMS template was aligned onto the DNA barcode microchip, and the microchip was taped into a 10 cm petri dish. The wells on the platform were wet with 50 μL PBST before filling with blocking buffer and placing into a 37° C. incubator for 1 hr. Concurrently, 40 μL 1% BSA in PBS solutions containing 750 nM of a SAC-DNA conjugate and 3.75 μM of one biotinylated PCC ligand or biotin-A₂₀-Cy3 blank were prepared for each SAC-DNA conjugate. The biotinylated ligands were allowed to complex with the SAC protein for one hour before pooling the SAC-DNA-ligand solutions (final [SAC-DNA-ligand conjugates]=50 nM). The blocking buffer was aspirated and each well was loaded with the SAC-DNA-ligand conjugates cocktail for hybridization with the DNA barcode at 37° C. for one hour. The wells were washed with PBST (3×) before loading serially diluted solutions of KRas protein in protein buffer (0 to 400 μM). After shaking at RT for one hour, the wells were rinsed with PBST (5×), making sure to pipet up/down with the first addition of PB ST. A (100:1) dilution of CST rabbit anti-Ras Ab (#39655) in 1° ab buffer was added to each well before shaking at RT for one hour. After rinsing the wells with PBST (3×), the wells were loaded with a (200:1) dilution of Abcam goat anti-rabbit-Alexafluor 647 linked ab (ab150079) in 2° ab buffer before shaking at RT for one hour. A final rinse of the wells with PBST (3×) proceeded peeling off the PDMS slab from the barcode microchip and dipping the barcode into the following solutions: PBS, (1:1) PBS: MQ H₂O, MQ H₂O (2×). After being spun dry, the barcode was read on the Genepix (635 nm, PMT 600, PWR 40% (60 W); 532 nm, PMT 450, PWR 15% (23 W)). Data was extracted using 10 ^{data blocks}/_{barcode lane}, double background corrected using the ab only well fluorescence and dummy ligand fluorescence in each well, and graphed in Graphpad (Sigmoidal 4PL mode with the Hill coefficient set=1). The peeled off PDMS slab was rinsed under MQ H₂O and stored in MQ H₂O at RT until further use.

### Measurement of PCC Ligand KRas EC₅₀ Using the Multi-Well ELISA Technology

### Buffers Used:

Blocking Buffer: TBS+5% milk+0.05% Tween20

Antibody (ab) Buffer: TBS, 5% BSA, 0.05% Tween20

Binding Buffer: TBS, 0.1% BSA, 0.05% Tween20

All steps were completed at room temperature

All wash steps used 200 ^{μL solution}/_{well}

All incubations used 100 ^{μL solution}/_{well} except for the 5% milk blocking step, which used 200 ^{μL solution}/_{well}

A 96-well Pierce Neutravidin Plate was washed with binding buffer (3×5 minutes at RT) before loading plate with a 1 μM solution of either blank (biotin-PEG₅-NHAc) (singly) or biotinylated PCC ligand (in triplicate). The plate incubated for two hours before washing with binding buffer (3×5 minutes). Blocking buffer was added to each well and the plate blocked for one hour before undergoing washing with binding buffer (3×5 minutes). Each well was loaded with either binding buffer or KRas solution (0→300 μM), and the plate was incubated for 30 minutes. Plate washing with binding buffer (3×5 minutes) preceded incubating the plate with a (1000:1) dilution of 1° antibody (ab) rabbit anti-Ras (CST #3965) in ab buffer for thirty minutes. The plate was washed with binding buffer (3×5 minutes), loaded with a (2000:1) dilution of 2° ab goat anti-rabbit, HRP-linked ab (CST #7074) in ab buffer, and incubated for an additional thirty minutes. The plate was rinsed with binding buffer (3×5 minutes), TBS (1×5 minutes), loaded with a (1:1) mixture of TMB Peroxidase Solution and TMB Peroxidase Solution B, and developed with occasional agitation for 8-12 minutes. After quenching the enzymatic reaction with 1M H₂SO_{4(aq)} (100 μL) the plate was read at λ=450 nm within ten minutes. The data was double background corrected using the ab only absorbance and the dummy ligand absorbance, plotted using Prism GraphPad 7 (Sigmoidal 4PL mode with the Hill coefficient set=1), and an EC₅₀ value was calculated.

### Intrinsic GTPase Activity

The GTPase assays were run in triplicate on a multi-well plate using the GTPase Glo Assay kit from Promega with 10 μM KRas protein and varying concentrations of ligand (1 μM to 100 μM L1a, L8 and 2.5 μM to 250 μM L2). All reagents were warmed to RT before use. A single opaque white 96-well plate was charged with 12.5 μL GTPase/GAP buffer (GTPase buffer w/ 1 mM DTT), 10 μM KRas in GTPase/GAP buffer, or 10 μM KRas protein with either L1a, L2, or L8. Running the survey assays on the same plate allowed for direct comparison of the curves, but it necessitated the use of the first row/column on the multi-well plate which introduced some noise to the low ligand concentration points. A 12.5 μL aliquot of a 2×GTP solution (10 μM rGTP in GTPase Buffer) was then added to each well before allowing the plate to shake at RT for two hours (initial GTPase inhibition assay). The GTPase Glo reagent was reconstituted in the GTPase Glo Buffer (4 μL GTPase Glo (500×) reagent, 1996 μL GTPase Glo Buffer, 1 μL 10 mM ADP) immediately before adding 25 μL to each well. Shaking at RT for thirty minutes preceded the addition of 50 μL of the Detection reagent to each well. The plate was covered and incubated for a total of ten minutes before reading the luminescence with a Flexstation 3 plate reader (All wavelengths mode, 500 ms integration time), graphed using Graphpad Prism 7 (Sigmoidal 4PL mode), and an IC₅₀ value was calculated.

For the full GTPase inhibition curve for L2, the above procedure was followed with the change that the incubation with KRas occurred over or four hours rather than two.

### Results and Discussion

The fabrication and assembly of the microchip platform for executing the invention is described in FIG. 5. A flow-patterning mold (50), fabricated from an elastomer, is mounted onto the surface of a polylysine coated glass slide. The mold has inputs (51) and outputs (52) for flowing through solutions that pass across the surface of the glass slide in serpentine fashion. One such pathway is illustrated (53). A different ssDNA oligomer (53) is flowed through every channel, and cross-linked to the polylysine coated (54) surface of the glass slide (55). The whole pattern may be developed (51) using a fluorophore-labelled ssDNA oligomers (56) that are complementary to the specific ssDNAs that are patterned in each channel (53). Once the DNA stripes (paths) have been patterned, the flow-patterning mold (50) is removed, and a second mold that defines a series of wells (57) is adhered to the patterned microscope slide surface. In this way, the serpentine pattern of ssDNA stripes (paths) is incorporated into a multi-well format (58). Fluorescent imaging of the stripes (paths) within an individual well (59) provides information regarding to the uniformity of the ssDNA patterning process.

### Optimizing B-RAP Technology Assay Conditions

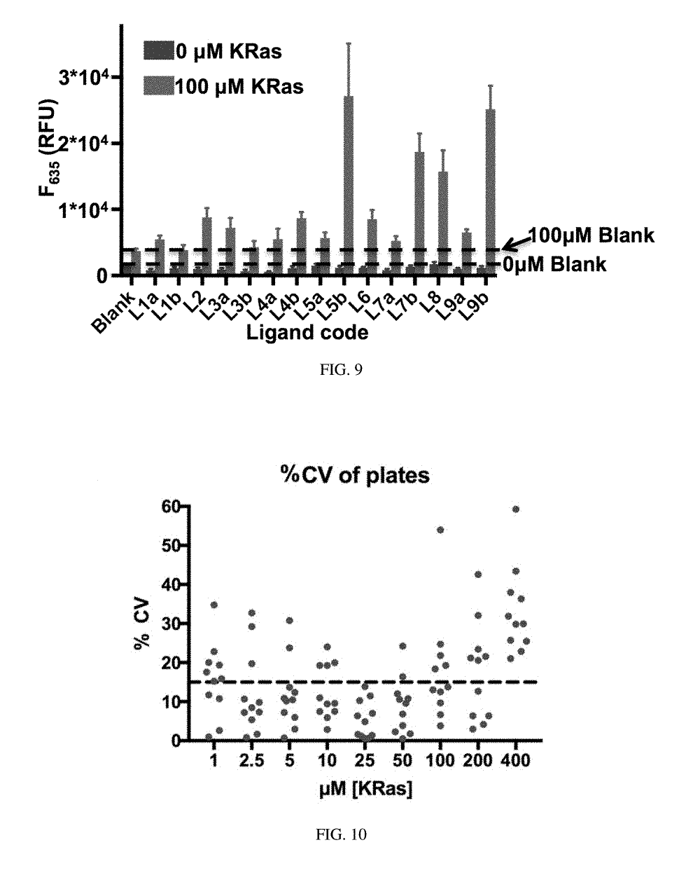

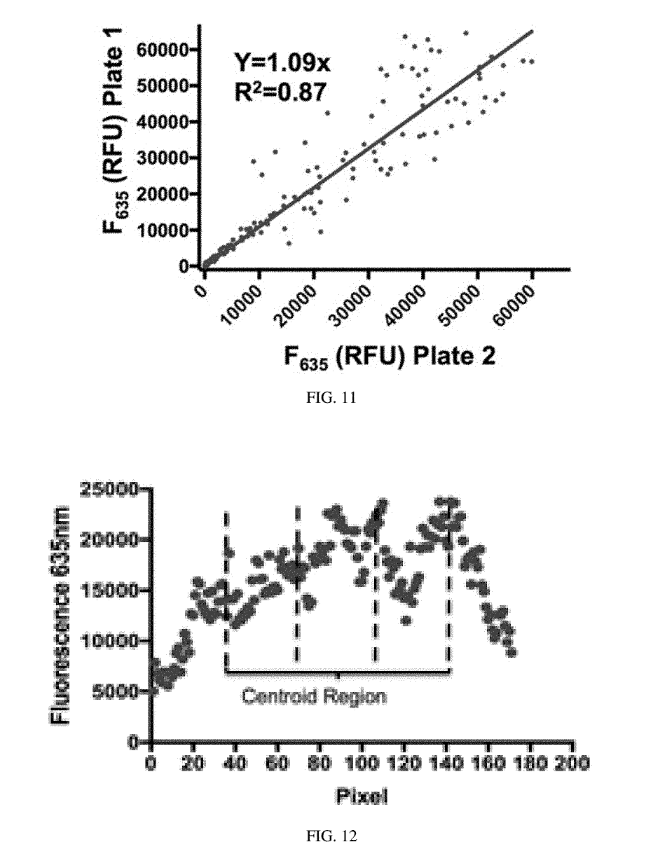

The in situ click screen against the Switch I and II KRas protein SynEps yielded five beads from which nine candidate sequences were determined (Table 9). Chromatographic purification yielded, for some ligands, identical mass peaks (possibly from epimerization), so that 15 candidate PCCs were isolated for testing. Biotinylated candidate ligands were then tested using a single-point IFA with the B-RAP technology (FIG. 9) to identify appropriate blocking conditions. Modification of the protein incubation solution to include the nonionic surfactant Polysorbate 20 (Tween20) was found to minimize non-specific binding between the KRas protein and the unmodified PLL surface.

Generally, H, K, and R and positively charged amino acids, F, W, and Y are aromatic amino acids, Q and T are polar amino acids, A, V, L, I, P, and G and neutral amino acids, and D and E are negatively charged amino acids.

TABLE 9
The hit sequences of the PCC candidates. Positions with high
homology exhibit pooling of similar types of amino acids at the
same positions. (a) - Similar sequences can arise from a single
hit bead due to uncertainty in the Edman degradation peptide
sequencing. b - These ligands had two correct mass fractions
following HPLC purification, arising from either epimerization
or differential protonation. Both fractions were tested.

| Bead (a) | Ligand | X1 | X2 | X3 | X4 | X5 |
|---|---|---|---|---|---|---|
| 1 | L1b | H | G | I | V | G |
| 2 | L2 | E | G | V | P | V |
| | L3b | G | E | V | V | P |
| 3 | L4b | V | E | V | P | Y |
| | L5b | V | E | V | Y | P |
| 4 | L6 | V | T | V | P | Y |
| | L7b | V | Q | V | P | Y |
| 5 | L8 | T | D | N | F | Y |
| | L9b | Q | D | N | F | Y |

### Validation of the B-RAP Technology

Following optimization of the assay conditions the B-RAP technology was subjected to statistical tests to assess the variance in assay results measured within an individual microwell, between microwells on the same chip, and between different microchips. The average percent coefficient of variation (% CV) seen along an individual barcode stripe in the wells above background (500 nM to 400 μM KRas) using the values from the data-block extraction method was ˜15%. Each microwell contains between two to three full copies of the DNA barcode. For the same barcode lane in the different full barcode sets in the same microwell, the fluorescence output was measured to have an average % CV of ˜14%. The results showed that extracting data from a single barcode repeat is sufficient. The % CV between wells on the same microchip run under identical conditions was ˜9%. The average % CV for identical barcode lanes between two separate platforms run in parallel by different users was ˜18% with an average % CV of ˜15% for the 1 μM to 400 μM range of KRas protein (FIG. 10). Additionally, to validate that our data-block extraction method of a portion of the barcode lane captured the F₆₃₅ for the entire barcode lane the average F₆₃₅ from a full-line line scan of the barcode lane was compared to the average F₆₃₅ resulting from our data-block extraction method. The values from the full-line scan were contained within two standard deviations of the data-block extraction's average F₆₃₅. This was compared to taking the measurement of individual pixels along the entirety of the barcode lanes in one full set of the 10 μM well for one plate then graphing to find the centroid region (FIG. 12), which is the region that is roughly stable in fluorescent intensity. The average F₆₃₅, the standard deviation, and the % CV for each lane was calculated for the full lane, the centroid region of the lane, and the different parts of the centroid region (Table 6). The full lane % CVs were in the 20-30% range, while the % CVs of the centroid regions were 10-20%. This arises from edge effects near the microwell walls. Assays of individual PCC candidates (different barcode stripes) collected within a single microwell, and so representing a single point of a binding curve, could be readily distinguished (Table 7). A scatterplot of the F₆₃₅ extracted for individual pixels along the entire length of a barcode in the 10 μM well of plate #1 showed some lanes that were indistinguishable by a two-tailed student T test. A scatterplot of the F₆₃₅ extracted for individual pixels in the centroid region of each barcode with lanes also showed some lanes that were indistinguishable by a two-tailed student T test (Table 7). These results indicated that the centroid region of a barcode stripe yielded the most reliable data, but also that assay results from different microwells, or different B-RAP chips, could be readily compared.

TABLE 6
A pixel by pixel analysis of variance along a barcode lane. The % CV values for the
entire barcode set using the full lane, full centroid, left part of the centroid,
middle part of the centroid, and right part of the centroid are displayed in the
top table. The average % CV values for each set are displayed in the bottom table.

| | | |
|---|---|---|
| Full Lane | Full Centroid | Left Part Centroid |

| Ligand | Avg F₆₃₅ | Std-dev | % CV | Avg F₆₃₅ | Std-dev | % CV | avg F635 | Std-dev | % CV |
|---|---|---|---|---|---|---|---|---|---|
| L6 | 15968 | 4436 | 27.8 | 17836 | 3037 | 17.0 | 15645 | 2058 | 13.2 |
| L4a | 10434 | 3093 | 29.6 | 11363 | 2491 | 21.9 | 10179 | 1444 | 14.2 |
| L4b | 25079 | 5502 | 21.9 | 27245 | 4173 | 15.3 | 23307 | 3555 | 15.3 |
| L7 | 48537 | 9262 | 19.1 | 53496 | 5310 | 9.9 | 48599 | 4548 | 9.4 |
| L3a | 7151 | 2185 | 30.6 | 8117 | 1291 | 15.9 | 7137 | 1106 | 15.5 |
| L3b | 15157 | 3057 | 20.2 | 16158 | 2040 | 12.6 | 15070 | 1916 | 12.7 |
| L5a | 7851 | 2139 | 27.2 | 8341 | 1748 | 21.0 | 7918 | 1268 | 16.0 |
| L5b | 31581 | 5350 | 16.9 | 32390 | 2859 | 8.8 | 31947 | 2750 | 8.6 |
| L7a | 32992 | 7831 | 23.7 | 36651 | 5433 | 14.8 | 32020 | 2774 | 8.7 |
| L7b | 14791 | 4605 | 31.1 | 16195 | 3322 | 20.5 | 12956 | 1819 | 14.0 |
| L9a | 16612 | 4630 | 27.9 | 18391 | 3310 | 18.0 | 14966 | 1107 | 7.4 |
| L9b | 41812 | 7828 | 18.7 | 45500 | 5838 | 12.8 | 39380 | 2637 | 6.7 |
| L1a | 18768 | 3903 | 20.8 | 20134 | 2815 | 14.0 | 17838 | 2497 | 14.0 |
| L1b | 22519 | 4786 | 21.3 | 24340 | 3430 | 14.1 | 21889 | 1763 | 8.1 |
| Blank | 14498 | 3225 | 22.2 | 15102 | 2350 | 15.6 | 12609 | 891 | 7.1 |

| | |
|---|---|
| Middle Part Centroid | Right Part Centroid |

| Ligand | avg F635 | Std-dev | % CV | avg F635 | Std-dev | % CV |
|---|---|---|---|---|---|---|
| L6 | 19382 | 2578 | 13.3 | 18546 | 3072 | 16.6 |
| L4a | 11181 | 2208 | 19.8 | 12764 | 2935 | 23.0 |
| L4b | 27398 | 2128 | 7.8 | 31147 | 2030 | 6.5 |
| L7 | 54620 | 3690 | 6.8 | 57411 | 3113 | 5.4 |
| L3a | 8430 | 952 | 11.3 | 8811 | 1171 | 13.3 |
| L3b | 16390 | 2085 | 12.7 | 17046 | 1615 | 9.5 |
| L5a | 8920 | 1975 | 22.1 | 8196 | 1826 | 22.3 |
| L5b | 32789 | 2700 | 8.2 | 32448 | 3133 | 9.7 |
| L7a | 37152 | 4709 | 12.7 | 40918 | 4399 | 10.8 |
| L7b | 16944 | 1899 | 11.2 | 18781 | 2914 | 15.5 |
| L9a | 20262 | 2145 | 10.6 | 20045 | 3023 | 15.1 |
| L9b | 47791 | 5200 | 10.9 | 49508 | 3078 | 6.2 |
| L1a | 20725 | 2225 | 10.7 | 21906 | 1983 | 9.1 |
| L1b | 24245 | 2629 | 10.8 | 26959 | 3580 | 13.3 |
| Blank | 15486 | 1525 | 9.8 | 17285 | 1505 | 8.7 |

| Region | average % CV |
|---|---|
| Full Lane | 23.9 |
| Full centroid | 15.5 |
| left part centroid | 11.4 |
| middle centroid | 11.9 |
| right part centroid | 12.3 |

TABLE 7
Calculated p-values for the pixel by pixel full-lane and centroid scatterplots. Symmetric 2-tail, unequal
variance p-value matrixes for the centroid and full-lane scatterplots (close-up shown in FIG. 12).

| | L6 | L4a | L4b | L7 | L3a | L3b | L5a | L5b | L7a | L7b | L9a | L9b | L1a | L1b | Blank |
|---|---|---|---|---|---|---|---|---|---|---|---|---|---|---|---|
| centroid | | | | | | | | | | | | | | | |
| L6 | — | *** | *** | *** | *** | *** | *** | *** | *** | *** | n.s. | *** | *** | *** | *** |
| L4a | | — | *** | *** | *** | *** | *** | *** | *** | *** | *** | *** | *** | *** | *** |
| L4b | | | — | *** | *** | *** | *** | *** | *** | *** | *** | *** | *** | *** | *** |
| L7 | | | | — | *** | *** | *** | *** | *** | *** | *** | *** | *** | *** | *** |
| L3a | | | | | — | *** | n.s. | *** | *** | *** | *** | *** | *** | *** | *** |
| L3b | | | | | | — | *** | *** | *** | n.s. | *** | *** | *** | *** | ** |
| L5a | | | | | | | — | *** | *** | *** | *** | *** | *** | *** | *** |
| L5b | | | | | | | | — | *** | *** | *** | *** | *** | *** | *** |
| L7a | | | | | | | | | — | *** | *** | *** | *** | *** | *** |
| L7b | | | | | | | | | | — | *** | *** | *** | *** | ** |
| L9a | | | | | | | | | | | — | *** | *** | *** | *** |
| L9b | | | | | | | | | | | | — | *** | *** | *** |
| L1a | | | | | | | | | | | | | — | *** | *** |
| L1b | | | | | | | | | | | | | | — | *** |
| blank | | | | | | | | | | | | | | | — |
| Full lane | | | | | | | | | | | | | | | |
| L6 | — | *** | *** | *** | *** | * | *** | *** | *** | * | n.s. | *** | *** | *** | ** |
| L4a | | — | *** | *** | *** | *** | *** | *** | *** | *** | *** | *** | *** | *** | *** |
| L4b | | | — | *** | *** | *** | *** | *** | *** | *** | *** | *** | *** | *** | *** |
| L7 | | | | — | *** | *** | *** | *** | *** | *** | *** | *** | *** | *** | *** |
| L3a | | | | | — | *** | ** | *** | *** | *** | *** | *** | *** | *** | *** |
| L3b | | | | | | — | *** | *** | *** | n.s. | ** | *** | *** | *** | n.s. |
| L5a | | | | | | | — | *** | *** | *** | *** | *** | *** | *** | *** |
| L5b | | | | | | | | — | n.s. | *** | *** | *** | *** | *** | *** |
| L7a | | | | | | | | | — | *** | *** | *** | *** | *** | *** |
| L7b | | | | | | | | | | — | *** | *** | *** | *** | n.s. |
| L9a | | | | | | | | | | | — | *** | *** | *** | *** |
| L9b | | | | | | | | | | | | — | *** | *** | *** |
| L1a | | | | | | | | | | | | | — | *** | *** |
| L1b | | | | | | | | | | | | | | — | *** |
| Blank | | | | | | | | | | | | | | | — |

The p-values are dentoted:
n.s. p > 0.05,
* 0.05 < p < 0.005,
** 0.005 < p < 0.0005,
*** p < 0.0005.

### Measuring the EC₅₀ of the Allosteric Binding PCC Ligands

After characterizing the B-RAP technology, we used the platform to generate complete binding curves for 15 PCC ligand fractions simultaneously (FIG. 6C), and determined the EC₅₀ values for each (Table 10) (for goodness of fit measurements for the curves see Table 8). These measurements were comprised of a 13-point concentration series, with each point collected in decaplicate. The EC₅₀ values enabled the ranking of the ligands, and the best binders were identified to be L1, L2, and L8. The true amino acid sequences for each hit peptide were also distinguished from the artifact sequences that arose from sequencing uncertainties. The true on-bead sequences for the hit beads are identified as L1, L2, L5, L7, and L8.

TABLE 10
The EC₅₀ values derived from the B-RAP technology and the multi-well ELISA technology.

| Ligand | L1a | L1b | L2 | L3a | L3b | L4a | L4b | L5a |
|---|---|---|---|---|---|---|---|---|
| B-RAP EC₅₀ | 0.97 ± 0.21 | 1.33 ± 0.28 | 3.31 ± 0.57 | 1.49 ± 0.28 | 13.65 ± 4.8 | 9.51 ± 2.2 | 52.24 ± 22 | 5.23 ± 0.70 |
| (μM) | | | | | | | | |
| ELISA EC₅₀ | 1.65^{b} ± 5.4 | 0.76^{b} ± 29 | 33.19^{b} ± 144 | 7.78 ± 10 | N.C.^{a} | N.C.^{a} | N.C.^{a} | 3.72^{b} ± 12 |
| (μM) | | | | | | | | |

| Ligand | L5b | L6^{a} | L7a | L7b | L8 | L9a | L9b |
|---|---|---|---|---|---|---|---|
| B-RAP EC₅₀ | 7.80 ± 2.3 | 18.88 ± 4.2 | 4.22 ± 0.71 | 25.29 ± 8.9 | 2.80 ± 0.42 | 5.43 ± 1.0 | 3.86 ± 0.57 |
| (μM) | | | | | | | |
| ELISA EC₅₀ | 6.87 ± 7.8 | 159^{b} ± 360 | 25.82 ± 14 | 44.16 ± 18 | 25.64 ± 8.3 | 2.18 ± 2.7 | 3.00 ± 4.9 |
| (μM) | | | | | | | |

^{a}Not calculated due to non-saturation of graph.
^{b}Select ligands that had the uncertainty for their EC₅₀ values greater than twice their EC₅₀ value and thus their binding curves were considered poorly resolved by the multi-well ELISA.

TABLE 8
The goodness of fit measurements for the allosteric KRas binding curves.

| | | | | | | | | | | | | | | | | BN-A₂₀₋ |
|---|---|---|---|---|---|---|---|---|---|---|---|---|---|---|---|---|
| | L1a | L1b | L2 | L3a | L3b | L4a | L4b | L5a | L5b | L6 | L7a | L7b | L8 | L9a | L9b | Cy3 |
| (a) | 0.91 | 0.92 | 0.95 | 0.94 | 0.81 | 0.91 | 0.8 | 0.97 | 0.85 | 0.92 | 0.95 | 0.85 | 0.96 | 0.94 | 0.96 | N/A |
| (b) | 0.95 | 0.95 | 0.96 | 0.96 | 0.83 | 0.93 | 0.87 | 0.98 | 0.88 | 0.94 | 0.97 | 0.89 | 0.98 | 0.95 | 0.97 | 0.66 |
| (c) | 0.56 | 0.18 | 0.45 | 0.73 | N/A | N/A | N/A | 0.53 | 0.78 | 0.73 | 0.93 | 0.96 | 0.97 | 0.81 | 0.72 | N/A |

The r-squared values for the curves after a double background correction (a) in FIG. 6C, (b) in an assay similar to that in FIG. 6C except without dummy ligand extraction, and (c) in the results of a 96 well ELISA assay.

TABLE 2
The EC50 values (62) from the fitted curves of FIG. 6B. The
values fall into the 5 to 60 microMolar range, as expected.

| | Candidate Ligands | | | | | | |
|---|---|---|---|---|---|---|---|
| | VTVPY | VEVPY f1 | VEVPY f2 | TDNFY | GEVVP f1 | GEVVP f2 | EGVPV |
| SEQ ID NO | 40 | 38 | 38 | 41 | 37 | 37 | 36 |
| EC50 | 16.02 μM | 19.56 μM | 32.72 μM | 8.44 μM | 53.31 μM | 19.37 μM | 8.19 μM |

| | Candidate Ligands | | | | | |
|---|---|---|---|---|---|---|
| | VQVPY f1 | VQVPY f2 | QDNFY f1 | QDNFY f2 | HGIVG f1 | HGIVG f2 |
| SEQ ID NO | 41 | 41 | 43 | 43 | 35 | 35 |
| EC50 | 31.46 μM | 30.32 μM | 6.55 μM | 6.29 μM | 16.18 μM | 12.21 μM |

We also provide a comparison of the EC₅₀ values from multi-well ELISA assays (triplicate measurements). While both assays identify L1a and L1b as the strongest binders, the ELISA assays are significantly noisier, with binding saturation not achieved for several ligands. The poor relative performance of the ELISAs arises from a few factors. First, the ligands tested are relatively weak (μM-level) binders, and this exacerbates certain issues associated with the ELISAs. ELISAs are absorbance measurements, and thus have a significantly smaller dynamic range than the B-RAP fluorescence assays. Second, ELISA signal arises from enzymatic amplification, while the B-RAP assays are not amplified. For weak binders, amplified assays tend to be noisy, as both signal and noise are amplified. The improved relative sensitivity and statistics afforded by the B-RAP technology readily enables the comparative evaluation of these relatively weak KRas binders.

### Testing the Allosteric Ligands as Inhibitors of KRas GTPase Activity

The ligands identified here were screened for binding to epitopes that exhibit structural fluctuations as the KRas protein switches between its inactive 5′-guanosine diphosphate (GDP)-bound form and its active GTP-bound form (Hall et al., J. Biol. Chem. 2001, 276 (29), 27629-27637). Consequently, the best three ligand fractions L1a, L2, and L8 were probed in a functional, solution phase assay for their ability to disrupt the intrinsic GTPase enzymatic activity of KRas protein. This assay measures the enzymatic conversion of GTP to GDP by KRas—a process that can potentially be inhibited. After incubation, an added GTPase Glo™ reagent converts the remaining GTP to ATP, and the ATP is converted into a chemiluminescent signal. Thus, higher chemiluminescence translates to lower KRas enzymatic activity. For the measurements, a fixed [KRas protein] is incubated with varying ligand concentrations. A concentration of 10 μM KRas protein was selected after generating a standard curve for the intrinsic KRas GTPase activity. KRas is a slow acting enzyme, so a KRas/PCC incubation time of two hours was used for the initial survey scans, while four hour incubation times were used for the higher resolution data. The survey assays indicated that all three ligands exhibited an inhibitory effect on the KRas protein's GTPase activity, but L2 was the most potent. Thus, the modulation of KRas activity by L2 was recorded with an expanded concentration range. We found that L2 switches from weakly activating to strongly inhibiting above 20 μM.

Less than 5% of the rGTP was hydrolyzed in the L2-only (no KRas) wells, and ˜61% was hydrolyzed in the KRas-only wells. This result confirms that L2 lacks any innate GTPase enzymatic activity. The sharp transition in the titration curve fits to a Hill coefficient of ˜10, and suggests that upon full occupancy of the allosteric switch region, KRas flips into an inactive conformation. The fitted IC₅₀ value was 24.0±1.2 μM for L2. This is an excellent starting point for a first generation allosteric inhibitor against this challenging target.

### CONCLUSIONS

We used the B-RAP technology to analyze the hits from an epitope targeted in situ click screen against the Kirsten rat sarcoma (KRas) protein (Cooper, Science 1982, 217 (4562), 801-806). Oncoprotein variants of KRas are implicated in driving ˜20-25% of all human cancers including almost all pancreatic cancers (Cox et al., Nature Reviews Drug Discovery. 2014, pp 828-851). Oncogenic Ras proteins have largely evaded targeting by traditional therapeutic techniques (Whitehead et al., Invest. New Drugs 2006, 24, 335-341; Macdonald et al., Invest. New Drugs 2005, 23, 485-487; Winquist et al., Urol. Oncol. 2003, 23 (3), 143-149; Sharma et al., Ann. Oncol. 2002, 13, 1067-1071), but recent work has shown that specific mutant isoforms may be targetable (Ostrem et al., Nature 2013, 503; Sakamoto et al., Biochem. Biophys. Res. Commun. 2017, 484 (3), 605-611). We targeted conserved epitopes denoted Switch I (aa 25-40) and Switch II (aa 56-75), which are known to allosterically influence KRas activity (Hall et al., J. Biol. Chem. 2001, 276 (29), 27629-27637). To our knowledge, these allosteric regions have not previously been targeted, perhaps because there is no obvious hydrophobic binding pocket. After screening, we tested the resultant hit compounds for their relative binding strengths. The strongest binders were then tested in a functional assay for in vitro KRas GTPase activity inhibition.

The disclosed barcoded rapid assay microchip allows for the simultaneous evaluation of a full set of PCC candidate ligands in, (in this example) up to sixteen unique assay conditions, with significant associated savings in terms of both time and reagent use (Table 1). In a single day the B-RAP technology was applied to identify the best allosteric KRas binders from a pool of 15 ligands identified from a dual SynEp PCC library in situ click screen. The B-RAP technology is designed to yield an equilibrium-based EC₅₀ value for assessing relative binding strengths. For a number of PCCs, the EC₅₀ value provides an upper limit for the dissociation constant (K_{D}) (Das et al., Angew. Chemie Int. Ed. 2015, 54 (45), 13219-13224). Importantly, relative binding affinities can provide guidance for selecting ligands for further quantitative characterizations, such as the solution phase KRas activity assay explored here. To this end, the B-RAP technology works well. A comparison of the B-RAP assay metrics relative to standard 96-well plate ELISAs is presented (Table 1). Extending this platform to evaluating PCC binders, or other ligand classes, against new protein targets will work well, requiring only an optimization of both concentration ranges (determined by the candidate ligands) and blocking conditions (typically determined by the protein target) (Gibbs, J. ELISA Tech. Bull. Corning Inc. Life Sci. Kennebunk, Me. 2001, No. 3, 1-6).

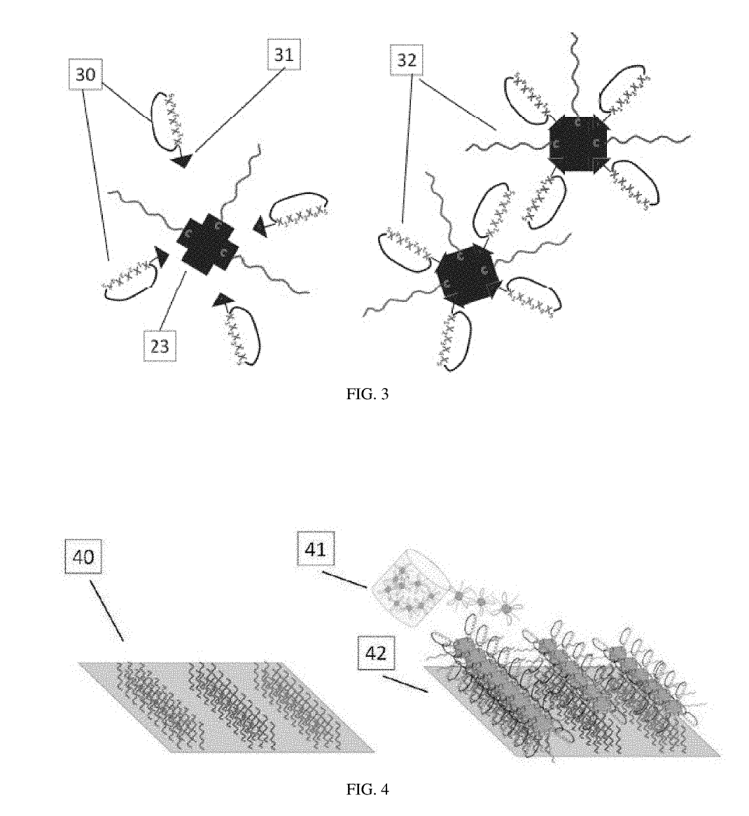

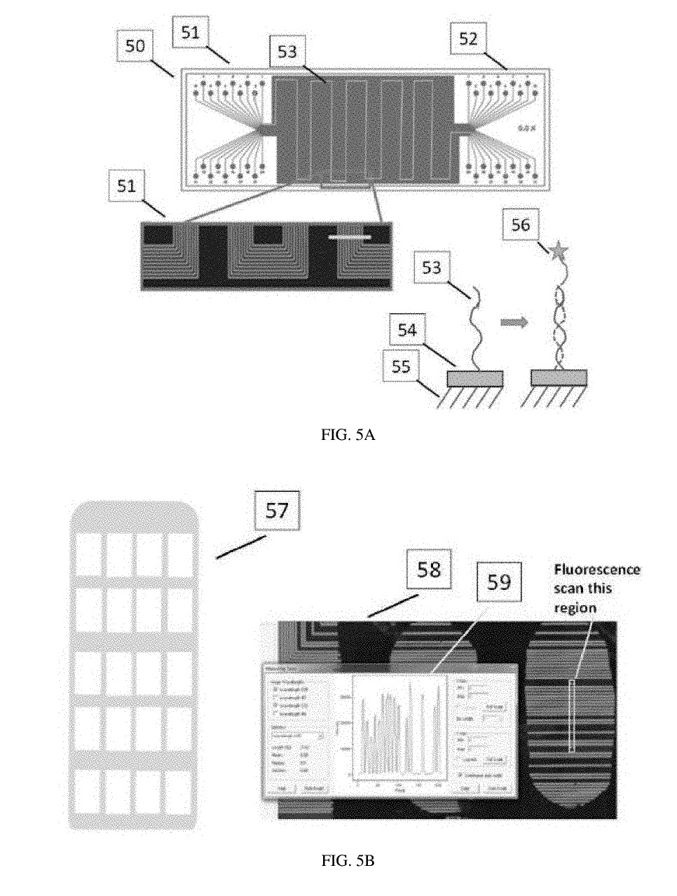

A series of peptide ligands that were identified as candidate binders to the protein KRas, using known literature methods, were prepared according to the methods of FIG. 3, and assembled into the individual microwells of the barcoded rapid assay microchip as illustrated in FIG. 4. Based upon more traditional multi-well plate assays, these peptide candidate ligands were anticipated to exhibit an EC₅₀ binding strength to the KRAS protein in the few micromolar range. Thus, binding curves were generated for the series of candidate ligands by using a different concentration of the KRAS protein in each of the wells of the barcoded rapid immunofluorescent assay microchip. Each assay was developed using an anti-KRAS antibody with a fluorophore label. Fluorescence imaging of the individual wells was then used to generate a fluorescence intensity, vs KRAS concentration, for each of the individual candidate ligands. Each well was designed to accommodate multiple barcode stripes (paths) for each of the candidate ligands, and so assay statistics (triplicate measurements) were generated for each point. The results are presented in FIG. 6.

The relative savings of time, reagents, plus the relative increases in data, when running the Barcoded Rapid Assay Microchip (BRAM) platform relative to the multi-well ELISA platform is summarized in Table 1.

TABLE 1
Comparison of the capacity, reagent quantities used,
and assay times for multi-well ELISA plates relative
to the barcoded rapid assay platform.

| | ELISA | B-RAP |
|---|---|---|
| Criteria | Platform | Chip |
| Full binding curves per Assay | 1 | 15 |
| Relative amount PCC per binding curve (nmol) | 7 | 0.15 |
| Relative amount protein per binding curve (nmol) | 300 | 2.7 |
| Protein concentration points per assay | 12 | 16 |
| Assay run time (h) | 10-36 | 8-10 |
| # Data Points per platform | 96 | 2400 |

Using the B-RAP platform coupled with the epitope-targeted in situ click screening approach, we identified a PCC ligand lead (L2) that serves as an allosteric inhibitor of the intrinsic GTPase enzymatic activity of KRas, with an IC₅₀ value of around 20 μM. L2 is a first generation ligand, and, as such, can surely be optimized, via medicinal chemistry methods, for increased potency and selectivity. Thus, given the well-known challenging nature of KRas as a drug target, L2 provides an excellent starting point for developing a more potent inhibitor.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a candidate molecule” includes a plurality of such candidate molecules, reference to “the candidate molecule” is a reference to one or more candidate molecule and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different candidate molecules does not indicate that the listed candidate molecules are obvious one to the other, nor is it an admission of equivalence or obviousness.

Every component disclosed herein is intended to be and should be considered to be specifically disclosed herein. Further, every subgroup or instance of a component that can be identified within this disclosure is intended to be and should be considered to be specifically disclosed herein. As a result, it is specifically contemplated that any component, or subgroup of components can be either specifically included for or excluded from use or included in or excluded from a list of components.

Those skilled in the art will recognize, or be able to ascertain using no more than routine experimentation, many equivalents to the specific embodiments of the method and compositions described herein. Such equivalents are intended to be encompassed by the following claims.

We claim:

1. A method comprising:
contacting a solid substrate with a plurality of labelled candidate molecules,
wherein the different labelled candidate molecules each comprise a different candidate molecule and a different label oligomer,
wherein the solid substrate comprises a plurality of positionally distinguishable, continuous paths, wherein each of a plurality of different substrate oligomers is attached to a different one of the paths, wherein each different label oligomer is complementary to a different one of the substrate oligomers,
wherein the label oligomers and the complementary substrate oligomers hybridize, wherein hybridization of a given label oligomer to the complementary substrate oligomer is bindingly distinguishable, wherein the hybridization results in localization of each different candidate molecule in each of the different paths; and
following or prior to contacting the solid substrate with the labelled candidate molecules, forming a plurality of test wells in the solid substrate, wherein one or more portions of each different path are in each well.

2. The method of claim 1, wherein each well exposes two or more different portions of each of the paths, wherein the two or more different portions of the paths are not continuous or contiguous in the well.

3. The method of claim 2, wherein each well exposes three different portions of each of the paths.

4. The method of claim 1, wherein the paths on the solid substrate change direction a plurality of times to form a serpentine pathway.

5. The method of claim 1, wherein one end of each path is proximal to a first side or edge of the solid substrate and the other end of each path is proximal to the side or edge of the solid substrate distal to the first side or edge of the solid substrate.

6. The method of claim 1, wherein one or more of the paths constitutes a control path, wherein no candidate molecule is localized in the control path.

7. The method of claim 6, wherein one or more of the control paths have a labelled control molecule localized in the control path, wherein the labelled control molecule is localized in the control path by, during the contacting step, contacting the solid substrate with the labelled control molecule,
wherein the labelled control molecule comprises a control molecule and a control label oligomer, wherein the control label oligomer is different from any of the label oligomers on the labelled candidate molecules localized on the solid substrate,
wherein the control label oligomer is complementary to one of the substrate oligomers, wherein the control label oligomer and the complementary substrate oligomer hybridize, resulting in localization of the control molecule in the path to which the complementary substrate oligomers is attached.

8. The method of claim 1, wherein the paths have a width of about 5 μm to about 100 μm.

9. The method of claim 8, wherein the paths have a pitch of about 1.5 times to about 3 times the width of the paths.

10. The method of claim 9, wherein the paths have a pitch of about 2 times the width of the paths.

11. The method of claim 8, wherein the width of the paths is 50 μm.

12. The method of claim 9, wherein the paths have a pitch of 100 μm.

13. The method of claim 1, wherein each well has an area of about 5 mm² to about 30 mm².

14. The method of claim 1, wherein each well has an area of about 18 mm².

15. The method of claim 1, wherein the length of the shortest line that crosses all of the different paths in a well is about 450 μm to about 18 mm².

16. The method of claim 1, wherein the length of the shortest line that crosses all of the different paths in a well is about 6 mm.

17. The method of claim 1, wherein the length of the shortest line that crosses the well is about 150 μm to about 6 mm.

18. The method of claim 1, wherein the length of the shortest line that crosses the well is about 3 mm.

19. The method of claim 1, wherein the ratio of the length of the shortest line that crosses all of the different paths in a well and the length of the shortest line that crosses the well is about 1 to about 5.

20. The method of claim 1, wherein the ratio of the length of the shortest line that crosses all of the different paths in a well and the length of the shortest line that crosses the well is about 3.

21. The method of claim 1, wherein the solid substrate is rectangular.

22. The method of claim 1, wherein the solid substrate comprises a glass slide or a plastic slide.

23. The method of claim 1 further comprising, prior to attachment of the substrate oligomers to the solid substrate, the solid substrate is coated with polylysine.

24. The method of claim 1, wherein the solid substrate comprises a bottom plate comprising a top surface, wherein the substrate oligomers are attached to the top surface of the bottom plate, wherein all of the paths are on the top surface of the bottom plate, wherein the plurality of wells are formed by adhering a top plate to the top surface of the bottom plate.

25. The method of claim 24, wherein the top plate comprises perforations, wherein the wells comprise the surface of the bottom plate exposed by the perforations in the top plate.

26. The method of claim 24, wherein the top plate is a microchannel mold comprising the wells, wherein the wells are chambers over the surface of the bottom plate.

27. The method of claim 24, wherein the bottom plate is rectangular.

28. The method of claim 24, wherein the bottom plate is a glass slide or a plastic slide.

29. The method of claim 24 further comprising, prior to attachment of the substrate oligomers to the solid substrate, the top plate is coated with polylysine.

30. The method of claim 1 further comprising, prior to contacting the solid substrate with the labelled candidate molecules and prior to forming the wells,
adhering a microchannel mold onto the solid substrate, wherein the adhered microchannel mold forms a different continuous sealed channel above each path on the solid substrate; and
flowing each different one of the substrate oligomers through a different formed channel and conjugating the substrate oligomers to the solid substrate.

31. The method of claim 30, wherein contacting the solid substrate with the labelled candidate molecules is accomplished by flowing the labelled candidate molecules through the formed channels.

32. The method of claim 31, wherein all of the labelled candidate molecules are flowed through each of the formed channels.

33. The method of claim 31, wherein each different one of the labelled candidate molecules is flowed through a different one of the formed channels.

34. The method of claim 30 further comprising, prior to forming the wells, removing the microchannel mold from the solid substrate.

35. The method of claim 30, wherein the microchannel mold is fabricated from an elastomer.

36. The method of claim 1, wherein the wells are formed prior to contacting the solid substrate with the labelled candidate molecules, wherein contacting the solid substrate with the labelled candidate molecules is accomplished by adding all of the labelled candidate molecules to each of the wells.

37. The method of claim 30, wherein contacting the solid substrate with the labelled candidate molecules is accomplished by adding all of the labelled candidate molecules to the solid substrate following removal of the microchannel mold and prior to forming the wells.

38. The method of claim 1, wherein contacting the solid substrate with the labelled candidate molecules is accomplished by adding all of the labelled candidate molecules to the solid substrate prior to forming the wells.

39. The method of claim 1 further comprising, following contacting the solid substrate with the labelled candidate molecules and to forming the wells:
adding an assay molecule to each well of the solid substrate, optionally excepting a control well,
adding an imaging agent to each well of the solid substrate, wherein the imaging agent binds to the assay molecule or to a product of the assay molecule, the candidate molecule, or the assay molecule and candidate molecule together,
detecting the imaging agent on a plurality of paths in each of a plurality of the wells.

40. The method of claim 39, wherein the imaging agent is detected in each of the paths in each of the wells.

41. The method of claim 39, wherein the imaging agent produces a fluorescent signal.

42. The method of claim 41, wherein the imaging agent produces a fluorescent signal upon excitation without the need for binding to or reaction with another molecule.

43. The method of claim 39, wherein the imaging agent is detected with a fluorescence image scanner.

44. The method of claim 43, wherein the image scanner generates a digitized output, wherein the digitized output is plotted as curves appropriate for the type of assay for each of the candidate molecules.

45. The method of claim 44, wherein the digitized output is plotted as binding curves for each of the candidate molecules.

46. The method of claim 39, wherein the imaging agent is detected in the middle third of the paths in the wells.

47. The method of claim 39, wherein a measured value of the detected imaging agent is produced by averaging the signals of the imaging agent detected at different points along the paths in the wells.

48. The method of claim 47, wherein a measured value of the detected imaging agent is produced for a given path in a given well by averaging the signals of the imaging agent detected at different points along the given path in the given well.

49. The method of claim 47, wherein a measured value of the detected imaging agent is produced for a given candidate molecule in a given well by averaging the signals of the imaging agent detected on the different paths for the given candidate molecule in the given well.

50. The method of claim 39, wherein the imaging agent comprises a fluorophore-labelled binding molecule.

51. The method of claim 39, wherein the imaging agent comprises a first binding molecule that binds to the assay molecule and a fluorophore-labelled binding molecule that binds to the first binding molecule.

52. The method of claim 51, wherein the first binding molecule is a primary antibody.

53. The method of claim 51, wherein the fluorophore-labelled binding molecule is a fluorophore-labelled antibody.

54. The method of claim 39, wherein the imaging agent comprises a fluorophore-labelled antibody.

55. The method of claim 54, wherein the imaging agent comprises a primary antibody that binds to the assay molecule, and a fluorophore-labelled antibody that binds to the primary antibody.

56. The method of claim 39, wherein a different concentration of the assay molecule is added to each well of the solid substrate.

57. The method of claim 39, wherein an interferant molecule is added to each well of the solid substrate, wherein the interferant molecule competes with the assay molecule for binding to the candidate molecules or inhibits reaction of the assay molecule with the candidate molecules.

58. The method of claim 57, wherein the interferant molecule is a competitive binding protein.

59. The method of claim 57, wherein a different concentration of the interferant molecule is added to each of the wells of the solid substrate.

60. The method of claim 1, wherein both the label oligomers and the substrate oligomers are ssDNA molecules.

61. The method of claim 1, wherein the labelled candidate molecules each further comprise a scaffold molecule, wherein the label oligomer of each labelled candidate molecule is chemically bonded to the scaffold molecule of the labelled candidate molecule and the candidate molecule of each labelled candidate molecule is bound or chemically bonded to the scaffold molecule of the labelled candidate molecule.

62. The method of claim 61, wherein the candidate molecule of each labelled candidate molecule is bound to the scaffold molecule of the labelled candidate molecule via a biotin-streptavidin interaction, wherein the scaffold molecule comprises streptavidin and the biotin is coupled to the candidate molecule.

63. The method of claim 61, wherein the label oligomer of each labelled candidate molecule is bound to the scaffold molecule of the labelled candidate molecule via a cysteine residue on the scaffold molecule.

64. The method of claim 61, wherein 1 to 10 copies of the same label oligomer are bonded to each scaffold molecule.

65. The method of claim 61, wherein 2 to 4 copies of the same label oligomer are bonded to each scaffold molecule.

66. The method of claim 61, wherein 4 copies of the same label oligomer are bonded to each scaffold molecule.

67. The method of claim 1, wherein the label oligomers are modified via succinimide chemistry to have a 5′-aminated oligonucleotide.

68. The method of claim 67, wherein a hydrazide moiety is introduced to the candidate molecules via reaction with an amino group, wherein a hydrazine bond forms between the hydrazide moiety of the candidate molecules and the 5′-aminated oligonucleotide of the label oligomers.

69. The method of claim 1, wherein the solid substrate comprises 10 paths to 30 paths.

70. The method of claim 1, wherein the solid substrate comprises 15 paths to 25 paths.

71. The method of claim 1, wherein the solid substrate comprises 20 paths.

72. The method of claim 1, wherein the solid substrate comprises 10 different candidate molecules to 30 different candidate molecules.

73. The method of claim 1, wherein the solid substrate comprises 15 different candidate molecules to 25 different candidate molecules.

74. The method of claim 1, wherein the solid substrate comprises 20 different candidate molecules.

75. A device for simultaneously testing a plurality of candidate molecules, the device comprising a solid substrate made by the method of claim 1.


SEQUENCE LISTING (42 sequences)

SEQ ID NO: 1 - DNA, 33 bp, Artificial Sequence
  Synthetic sequence
  NH2-C6- (1)..(1): 5′ amino C6 linker NH2-C6-
aaaaaaaaaaaaagcctcattgaatcatgccta

SEQ ID NO: 2 - DNA, 30 bp, Artificial Sequence
  Synthetic sequence
  NH2-C6- (1)..(1): 5′ amino C6 linker NH2-C6-
aaaaaaaaaataggcatgattcaatgaggc

SEQ ID NO: 3 - DNA, 33 bp, Artificial Sequence
  Synthetic sequence
  NH2-C6- (1)..(1): 5′ amino C6 linker NH2-C6-
aaaaaaaaaaaaagcactcgtctactatcgcta

SEQ ID NO: 4 - DNA, 30 bp, Artificial Sequence
  Synthetic sequence
  NH2-C6- (1)..(1): 5′ amino C6 linker NH2-C6-
aaaaaaaaaatagcgatagtagacgagtgc

SEQ ID NO: 5 - DNA, 30 bp, Artificial Sequence
  Synthetic sequence
  NH2-C6- (1)..(1): 5′ amino C6 linker NH2-C6-
aaaaaaaaaaaaggtcgagatgtcagagta

SEQ ID NO: 6 - DNA, 30 bp, Artificial Sequence
  Synthetic sequence
  NH2-C6- (1)..(1): 5′ amino C6 linker NH2-C6-
aaaaaaaaaatactctgacatctcgaccat

SEQ ID NO: 7 - DNA, 33 bp, Artificial Sequence
  Synthetic sequence
  NH2-C6- (1)..(1): 5′ amino C6 linker NH2-C6-
aaaaaaaaaaaaaatgtgaagtggcagtatcta

SEQ ID NO: 8 - DNA, 30 bp, Artificial Sequence
  Synthetic sequence
  NH2-C6- (1)..(1): 5′ amino C6 linker NH2-C6-
aaaaaaaaaatagatactgccacttcacat

SEQ ID NO: 9 - DNA, 33 bp, Artificial Sequence
  Synthetic sequence
  NH2-C6- (1)..(1): 5′ amino C6 linker NH2-C6-
aaaaaaaaaaaaaatcaggtaaggttcacggta

SEQ ID NO: 10 - DNA, 30 bp, Artificial Sequence
  Synthetic sequence
  NH2-C6- (1)..(1): 5′ amino C6 linker NH2-C6-
aaaaaaaaaataccgtgaaccttacctgat

SEQ ID NO: 11 - DNA, 33 bp, Artificial Sequence
  Synthetic sequence
  NH2-C6- (1)..(1): 5′ amino C6 linker NH2-C6-
aaaaaaaaaaaaagagtagccttcccgagcatt

SEQ ID NO: 12 - DNA, 30 bp, Artificial Sequence
  Synthetic sequence
  NH2-C6- (1)..(1): 5′ amino C6 linker NH2-C6-
aaaaaaaaaaaatgctcgggaaggctactc

SEQ ID NO: 13 - DNA, 33 bp, Artificial Sequence
  Synthetic sequence
  NH2-C6- (1)..(1): 5′ amino C6 linker NH2-C6-
aaaaaaaaaaaaaattgaccaaactgcggtgcg

SEQ ID NO: 14 - DNA, 30 bp, Artificial Sequence
  Synthetic sequence
  NH2-C6- (1)..(1): 5′ amino C6 linker NH2-C6-
aaaaaaaaaacgcaccgcagtttggtcaat

SEQ ID NO: 15 - DNA, 33 bp, Artificial Sequence
  Synthetic sequence
  NH2-C6- (1)..(1): 5′ amino C6 linker NH2-C6-
aaaaaaaaaaaaatgccctattgttgcgtcgga

SEQ ID NO: 16 - DNA, 30 bp, Artificial Sequence
  Synthetic sequence
  NH2-C6- (1)..(1): 5′ amino C6 linker NH2-C6-
aaaaaaaaaatccgacgcaacaatagggca

SEQ ID NO: 17 - DNA, 33 bp, Artificial Sequence
  Synthetic sequence
  NH2-C6- (1)..(1): 5′ amino C6 linker NH2-C6-
aaaaaaaaaaaaataatctaattctggtcgcgg

SEQ ID NO: 18 - DNA, 30 bp, Artificial Sequence
  Synthetic sequence
  NH2-C6- (1)..(1): 5′ amino C6 linker NH2-C6-
aaaaaaaaaaccgcgaccagaattagatta

SEQ ID NO: 19 - DNA, 33 bp, Artificial Sequence
  Synthetic sequence
  NH2-C6- (1)..(1): 5′ amino C6 linker NH2-C6-
aaaaaaaaaaaaagtgattaagtctgcttcggc

SEQ ID NO: 20 - DNA, 30 bp, Artificial Sequence
  Synthetic sequence
  NH2-C6- (1)..(1): 5′ amino C6 linker NH2-C6-
aaaaaaaaaagccgaagcagacttaatcac

SEQ ID NO: 21 - DNA, 33 bp, Artificial Sequence
  Synthetic sequence
  NH2-C6- (1)..(1): 5′ amino C6 linker NH2-C6-
aaaaaaaaaaaaagtcgaggattcgtaacctgt

SEQ ID NO: 22 - DNA, 30 bp, Artificial Sequence
  Synthetic sequence
  NH2-C6- (1)..(1): 5′ amino C6 linker NH2-C6-
aaaaaaaaaaacaggttcagaatcctcgac

SEQ ID NO: 23 - DNA, 33 bp, Artificial Sequence
  Synthetic sequence
  NH2-C6- (1)..(1): 5′ amino C6 linker NH2-C6-
aaaaaaaaaaaaagtcctcgcttcgtctatgag

SEQ ID NO: 24 - DNA, 30 bp, Artificial Sequence
  Synthetic sequence
  NH2-C6- (1)..(1): 5′ amino C6 linker NH2-C6-
aaaaaaaaaactcatagacgaagcgaggac

SEQ ID NO: 25 - DNA, 33 bp, Artificial Sequence
  Synthetic sequence
  NH2-C6- (1)..(1): 5′ amino C6 linker NH2-C6-
aaaaaaaaaaaaacttcgtggctagtctgtgac

SEQ ID NO: 26 - DNA, 30 bp, Artificial Sequence
  Synthetic sequence
  NH2-C6- (1)..(1): 5′ amino C6 linker NH2-C6-
aaaaaaaaaagtcacagactagccacgaag

SEQ ID NO: 27 - DNA, 33 bp, Artificial Sequence
  Synthetic sequence
  NH2-C6- (1)..(1): 5′ amino C6 linker NH2-C6-
aaaaaaaaaaaaatcgccgttggtctgtatgca

SEQ ID NO: 28 - DNA, 30 bp, Artificial Sequence
  Synthetic sequence
  NH2-C6- (1)..(1): 5′ amino C6 linker NH2-C6-
aaaaaaaaaatgcatacagaccaacggcga

SEQ ID NO: 29 - DNA, 33 bp, Artificial Sequence
  Synthetic sequence
  NH2-C6- (1)..(1): 5′ amino C6 linker NH2-C6-
aaaaaaaaaaaaataagccagtgtgtcgtgtct

SEQ ID NO: 30 - DNA, 30 bp, Artificial Sequence
  Synthetic sequence
  NH2-C6- (1)..(1): 5′ amino C6 linker NH2-C6-
aaaaaaaaaagacacgacacactggcttat

SEQ ID NO: 31 - protein, 12 aa, Artificial Sequence
  Synthetic sequence
  Xaa (6)..(6): Xaa is 4-azidoproline residue
FDEYDXTIEDSY

SEQ ID NO: 32 - protein, 12 aa, Artificial Sequence
  Synthetic sequence
  Xaa (1)..(1): Xaa is a 4-azidoproline residue
XEYDSIDDEFYT

SEQ ID NO: 33 - protein, 11 aa, Artificial Sequence
  Synthetic sequence
  Xaa (1)..(1): Xaa is azidolysine residue
XLDTAGQEEYS

SEQ ID NO: 34 - protein, 11 aa, Artificial Sequence
  Synthetic sequence
  Xaa (1)..(1): Xaa is an azidolysine residue
XLSTGEYDAQE

SEQ ID NO: 35 - protein, 5 aa, Artificial Sequence
  Synthetic sequence
  misc_feature (1)..(5): L1
HGIVG

SEQ ID NO: 36 - protein, 5 aa, Artificial Sequence
  Synthetic sequence
  L2 (1)..(5)
EGVPV

SEQ ID NO: 37 - protein, 5 aa, Artificial Sequence
  Synthetic sequence
  L3 (1)..(5)
GEVVP

SEQ ID NO: 38 - protein, 5 aa, Artificial Sequence
  Synthetic sequence
  L4 (1)..(5)
VEVPY

SEQ ID NO: 39 - protein, 5 aa, Artificial Sequence
  Synthetic sequence
  L5 (1)..(5)
VEVYP

SEQ ID NO: 40 - protein, 5 aa, Artificial Sequence
  Synthetic sequence
  L6 (1)..(5)
VTVPY

SEQ ID NO: 41 - protein, 5 aa, Artificial Sequence
  Synthetic sequence
  L7 (1)..(5)
VQVPY

SEQ ID NO: 42 - protein, 5 aa, Artificial Sequence
  Synthetic sequence
  L8 (1)..(5)
TDNFY